(19) United States
(12) Patent Application Publication
(10) Pub. No.: US 2024/0287078 A1
(43) Pub. Date: Aug. 29, 2024

(54) QUINAZOLINE DERIVATIVES USEFUL AS RAS INHIBITORS

(71) Applicant: Redx Pharma PLC, Macclesfield Cheshire (GB)

(72) Inventors: Clifford D. Jones, Macclesfield Cheshire (GB); Inder Bhamra, Macclesfield Cheshire (GB); James Ryan, Macclesfield Cheshire (GB)

(21) Appl. No.: 18/565,009

(22) PCT Filed: Jun. 9, 2022
(86) PCT No.: PCT/GB22/51446

§ 371 Date: Nov. 28, 2023

(30) Foreign Application Priority Data
Jun. 10, 2021 (GB) 2108334.0
Dec. 21, 2021 (GB) 2118633.3

(51) Int. Cl. C07D 487/04; A61K 31/517; A61K 31/541; A61K 31/551; A61K 31/553; A61K 31/554; C07D 519/00

(52) U.S. Cl. CPC C07D 487/04; A61K 31/517; A61K 31/541; A61K 31/551; A61K 31/553; A61K 31/554; C07D 519/00

(57) ABSTRACT

This invention relates to compounds that may be useful for inhibiting RAS proteins. More specifically, this invention relates to compounds for inhibiting a broad spectrum of KRAS mutant proteins. The compounds of the invention may therefore be used in treating conditions mediated by KRAS proteins. For example, the compounds may be used in treating cancer.

This invention relates to compounds and their methods of use. In particular, the compounds of the present invention may be useful for inhibiting RAS proteins. More specifically, this invention relates to compounds for inhibiting a broad spectrum of KRAS proteins including mutant strains and wild-type KRAS. The compounds of the invention may therefore be used in treating conditions mediated by KRAS proteins. For example, the compounds may be used in treating cancer.

### BACKGROUND

RAS (HRAS, KRAS4A and 4B, and NRAS) proteins are a group of closely related monomeric globular proteins that act as molecular switches, cycling between inactive (GDP-bound) and active (GTP-bound) states to transduce upstream cellular signals to downstream effectors to regulate a wide variety of processes, including cellular proliferation. RAS is the most frequently mutated oncogene in cancer (˜30%), with KRAS the most commonly mutated isoform accounting for ˜85% of RAS mutations (Hobbs et al, Journal of Cell Science (2016) 129, 1287-1292 doi:10.1242/jcs.182873).

KRAS G12D is a missense gain of function mutation that results in an amino acid substitution of the glycine (G) at codon 12 with aspartic acid and is the most prevalent accounting for ˜26% of all KRAS mutations in cancer. KRAS G12D mutations are present in 36% pancreatic carcinoma patients, 13% colorectal carcinoma patients, 10% rectal carcinoma patients, 6% endometrial carcinoma patients, 4% of non-small cell lung carcinoma patients, 4% gastric carcinoma patients, 3% ovarian carcinoma patients and 2% small cell lung carcinoma patients (e.g. The AACR Project GENIE Consortium, (2017) Cancer Discovery; 7(8):818-831. Dataset version 8). Many of these patients with G12D mutations have high unmet need with little option of efficacious targeted therapy. The mainstay of treatment for many of these patients remains chemotherapy combinations with an associated high degree of side effects and lack of efficacy.

Other KRAS missense gain of functions mutations that result in amino acid substitutions at codon 12, codon 13 and codon 61, as well as amplification of KRAS wildtype protein also drive carcinogenesis. Alterations in KRAS are found in approximately one in seven cancers (Hoffman et al, Cancer Discovery (2022) 12, 924-937). Activating mutations in KRAS are highly prevalent in solid tumours and are predominately found in 35% lung, 45% colorectal and up to 90% pancreatic cancers. G12D, G12V and G12C are the most frequently occurring KRAS mutations and are found more than half of all KRAS driven cancers. Other KRAS mutations include KRAS G12V, KRAS G12A, KRAS G13D and KRAS Q61H. KRAS amplifications are found in approximately 7% of cancers with KRAS alterations and are commonly occurring in ovarian carcinoma, breast carcinoma, lung adenocarcinoma, gastric adenocarcinoma, uterine cancers and esophagogastric cancers (Hoffman reviews). Pan KRAS inhibitors have the potential to treat a broader patient population including cancers harbouring KRAS mutations, KRAS wildtype amplifications and cancers driven by loss of the tumour suppressor NF1. In addition, pan KRAS inhibitors can potentially be used to treat cancers with acquired resistance to allele specific inhibitors such as KRAS G12C inhibitors.

Due to this frequency of KRAS mutations in multiple different tumour types and the established role of KRAS as an oncogenic driver mutation in cancer, modulating the activity of KRAS is a highly attractive therapeutic goal and has been the subject of significant research efforts for greater than 30 years. However, it has proven extremely challenging to affect KRAS activity directly and research efforts have focused on other targets in the signalling cascade that are either upstream or downstream from KRAS. Other approaches to inhibit KRAS activity have included affecting other points on the MAPK pathway (English et al., 2002; Adjei 2014; Chin et al., 2020), many of which have shown MAPK pathway inhibition to be clinically effective. Recently, mutant KRAS G12C selective inhibitors have been reported (Kettle and Cassar 2020), which bind covalently to an allosteric pocket and have progressed into clinical trials and shown responses in selected patients.

Compounds which are capable of modulating G12D mutant KRAS are described in WO2021/041671. Compounds capable of modulating multiple RAS isoforms and mutants have also been described (Kessler et al. 2019), however these compounds are believed to be of limited therapeutic benefit owing to a lack of sufficient potency as well as little selectivity for KRAS over HRAS and NRAS isoforms.

An aim of the present invention is to provide alternative or improved compounds for inhibiting RAS proteins. For example, an aim of the present invention is to provide alternative or improved compounds for inhibiting KRAS proteins.

Furthermore, it is an aim of certain embodiments of this invention to provide new compounds for use in treatment of conditions modulated by RAS proteins. For example, it is an aim of certain embodiments of this invention to provide compounds for use in the treatment of cancer. Said compounds may be more selective for KRAS proteins having the G12D mutation over alternative KRAS proteins than prior art compounds. Alternatively, said compounds may have broad spectrum activity across a range of KRAS proteins.

It is an aim of certain embodiments of this invention to provide new cancer treatments. In particular, it is an aim of certain embodiments of this invention to provide compounds which have comparable activity to existing treatments, optionally they should have better activity.

It is an aim of certain embodiments of this invention to provide compounds which exhibit reduced cytotoxicity relative to prior art compounds and existing therapies.

Another aim of certain embodiments of this invention is to provide compounds having a convenient pharmacokinetic profile and a suitable duration of action following dosing. A further aim of certain embodiments of this invention is to provide compounds in which the metabolised fragment or fragments of the drug after absorption are GRAS (Generally Regarded As Safe).

Certain embodiments of the present invention satisfy some or all of the above aims.

### BRIEF SUMMARY OF THE DISCLOSURE

In accordance with the present inventions there is provided a compound of formula (I), or a pharmaceutically acceptable salt thereof:

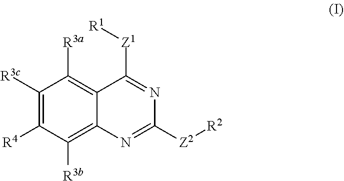

wherein
Z¹ is independently selected from —O— and —NR⁵—;
Z² is independently absent or is selected from —O— and —NR⁶—;
R¹ is independently selected from C₀-C₃-alkylene-R^{1a} and C₂-C₆-alkylene-R^{1b}; wherein R^{1a} is independently selected from an oxygen containing 4- to 7-membered heterocycloalkyl ring, a 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups; R^{1b} is independently selected from: NR⁷R⁸, OR⁸, SR⁸, SOR⁸, SO₂R⁸ and SO(NH)R⁸;
or R¹ and R⁵ together with the nitrogen to which they are attached form a ring system selected from: a monocyclic 4- to 7-membered heterocycloalkyl group, optionally substituted with from 1 to 4 R⁹ groups; a fused, spirofused or bridged bicyclic 6- to 11-membered heterocycloalkyl group, optionally substituted with from 1 to 4 R⁹ groups;
R² is independently C₁-C₆-alkyl, C₁-C₄-haloalkyl, C₀-C₄-alkylene-R^{2a}, C1-C₄-alkylene-R^{2b}, C₂-C₄-alkylene-R^{2c};
R^{2a} is independently selected from monocyclic 4- to 7-membered heterocycloalkyl group, a fused, spirofused or bridged bicyclic 6- to 11-membered heterocycloalkyl group; a 5-, 6-, 9- or 10-membered monocyclic or bicyclic heteroaryl group; phenyl; C₃-C₇-cycloalkyl; wherein any heterocycloalkyl or cycloalkyl R^{2a} group is optionally substituted with from 1 to 6 R¹⁰ groups and any heteroaryl or phenyl R^{2a} group is optionally substituted with from 1 to 6 R¹¹ groups;
wherein R^{2b} is independently selected from CONR¹²R¹² and CO₂R¹²;
wherein R^{2b} is independently selected from NR¹²R¹³ and OR¹²;
or R² and R⁶ together with the nitrogen to which they are attached form a ring system selected from: monocyclic 4- to 7-membered heterocycloalkyl group, a fused, spirofused or bridged bicyclic 6- to 11-membered heterocycloalkyl group, said heterocycloalkyl group being optionally substituted with from 1 to 6 R¹⁰ groups;
R^{3a}, R^{3b} and R^{3c} are each independently selected from: H, halo, C₁-C₄-alkyl, O—C₁-C₄-alkyl, C1-C₄-haloalkyl, O—C₁-C₄-haloalkyl, cyclopropyl, nitro and cyano;
R⁴ is independently selected from: phenyl, said phenyl being optionally fused to a C₅-C₇-cycloalkyl ring; naphthyl; and 5-, 6-, 9- or 10-membered monocyclic or bicyclic heteroaryl, wherein R⁴ is optionally substituted with from 1 to 4 R¹⁴ groups;
R⁵, R⁶, R⁸ and R¹² are each independently selected at each occurrence from H, C1-C₄-haloalkyl, and C₁-C₄-alkyl;
R⁷ and R¹³ are each independently at each occurrence selected from H, C₁-C₄-alkyl, C1-C₄-haloalkyl and C(O)—C₁-C₄-alkyl;
R⁹ and R¹⁰ are each independently at each occurrence selected from oxo, halo, cyano, NR¹²R¹³, OR¹², CO₂R¹², CONR¹²R¹², C₁-C₄-alkyl, C₁-C₄-alkyl substituted with NR¹²R¹³, C1-C₄-alkyl substituted with OR¹², C₁-C₄-alkyl substituted with cyano, C₂-C₄-alkenyl, C₂-C₄-alkynyl, C₁-C₄-haloalkyl and cyclopropyl;
R¹¹ and R¹⁴ are each independently at each occurrence selected from halo, cyano, nitro, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₂-C₄-alkenyl, C₂-C₄-alkynyl, C₁-C₄-haloalkyl and cyclopropyl;
wherein any of the aforementioned alkyl, alkylene or cyclopropyl groups is optionally substituted, where chemically possible, by 1 to 5 substituents which are each independently at each occurrence selected from the group consisting of: C₁-C₄-alkyl, halo, nitro, cyano, NR^{a}R^{b}, OR^{a}, SR^{a}, CO₂R^{a}, C(O)R^{a}, CONR^{a}R^{a}; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.

In an embodiment, the compound of formula (I) is a compound of formula (Ia):

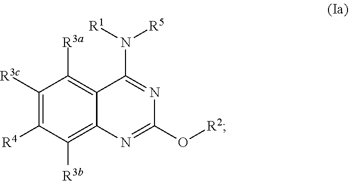

wherein
R¹ is independently selected from C₀-C₃-alkylene-R^{1a} and C₂-C₈-alkylene-R^{1b}; wherein R^{1a} is independently selected from a 4- to 7-membered heterocycloalkyl ring; a fused or spirofused bicyclic 6- to 11-membered heterocycloalkyl group, optionally substituted with from 1 to 4 R⁹ groups and a C₃-C₇-cycloalkyl ring; wherein said heterocycloalkyl ring or said cycloalkyl ring is optionally substituted with from 1 to 4 R⁹ groups; R^{1b} is independently selected from: OR⁸, SR⁸, SOR⁸, SO₂R⁸, SO(NH)R⁸, OC(O)R⁸, and SO₂NR⁷R⁸;
or R¹ and R⁵ together with the nitrogen to which they are attached form a ring system selected from: a monocyclic 4- to 7-membered heterocycloalkyl group, optionally substituted with from 1 to 4 R⁹ groups; a fused, spirofused or bridged bicyclic 6- to 11-membered heterocycloalkyl group, optionally substituted with from 1 to 4 R⁹ groups;
wherein R¹ and R⁵ are selected such that NR¹R⁵ comprises no more than a single amine,
wherein said single amine may be a primary, secondary or tertiary amine;
R² is independently C₁-C₈-alkyl, C₁-C₄-haloalkyl, C₀-C₄-alkylene-R^{2a}, C₁-C₄-alkylene-R^{2b}, C₂-C₄-alkylene-R^{2c};
R^{2a} is independently selected from monocyclic 4- to 7-membered heterocycloalkyl group, a fused, spirofused or bridged bicyclic 6- to 11-membered heterocycloalkyl group; a 5-, 6-, 9- or 10-membered monocyclic or bicyclic heteroaryl group; phenyl; C₃-C₇-cycloalkyl; wherein any heterocycloalkyl or cycloalkyl R^{2a} group is optionally substituted with from 1 to 6 R¹⁰ groups and any heteroaryl or phenyl R^{2a} group is optionally substituted with from 1 to 6 R¹¹ groups;
R^{2b} is independently selected from CONR¹²R¹² and CO₂R¹²;
R^{2b} is independently selected from NR¹²R¹³ and OR¹²
R^{3a}, R^{3b} and R^{3c} are each independently selected from: H, halo, C₁-C₄-alkyl, O—C₁-C₄-alkyl, C₁-C₄-haloalkyl, O—C₁-C₄-haloalkyl, cyclopropyl, nitro and cyano;
R⁴ is independently selected from: phenyl, said phenyl being optionally fused to a C₅-C₇-cycloalkyl ring; naphthyl; and 5-, 6-, 9- or 10-membered monocyclic or bicyclic heteroaryl, wherein R⁴ is optionally substituted with from 1 to 4 R¹⁴ groups;
R⁵, R⁸ and R¹² are each independently selected at each occurrence from H, C₁-C₄-haloalkyl, and C₁-C₄-alkyl;
R⁷ and R¹³ are each independently at each occurrence selected from H, C₁-C₄-alkyl, C₁-C₄-haloalkyl and C(O)—C₁-C₄-alkyl;
R⁹ and R¹⁰ are each independently at each occurrence selected from oxo, halo, cyano, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₁-C₄-alkyl substituted with cyano, C₂-C₄-alkenyl, C₂-C₄-alkynyl, C₁-C₄-haloalkyl and cyclopropyl;
R¹¹ and R¹⁴ are each independently at each occurrence selected from halo, cyano, nitro, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₂-C₄-alkenyl, C₂-C₄-alkynyl, C₁-C₄-haloalkyl and cyclopropyl; wherein any of the aforementioned alkyl, alkylene or cyclopropyl groups is optionally substituted, where chemically possible, by 1 to 5 substituents which are each independently at each occurrence selected from the group consisting of: C₁-C₄-alkyl, halo, nitro, cyano, NR^{a}R^{b}, OR^{a}, SR^{a}, CO₂R^{a}, C(O)R^{a}, CONR^{a}R^{a}; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.

In an embodiment, the compound of formula (I) is a compound of formula (II):

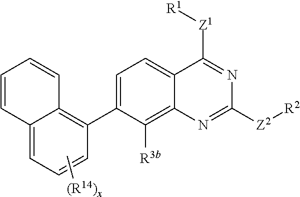

wherein R¹, R², R^{3b}, R¹⁴, Z¹ and Z² are as described above for compounds of formula (I); and x is independently selected from 0, 1, 2, 3, and 4. For the absence of doubt, throughout this specification, the x R¹⁴ groups may be attached to either ring of the naphthyl group.

In an embodiment, the compound of formula (I) is a compound of formula (IIa):

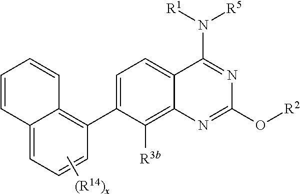

wherein R¹, R², R⁵, R^{3b}, R¹⁴ are as described above for compounds of formula (I); and x is independently selected from 0, 1, 2, 3, and 4. For the absence of doubt, throughout this specification, the x R¹⁴ groups may be attached to either ring of the naphthyl group.

In an embodiment, the compound of formula (I) is a compound of formula (III):

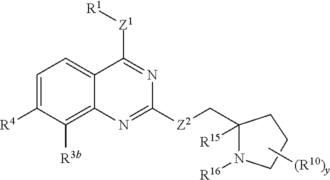

wherein R¹, R^{3b}, R⁴, R¹⁰, Z¹ and Z² are as described above for compounds of formula (I); and wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; and y is independently selected from 0, 1, 2, 3, and 4.

In an embodiment, the compound of formula (I) is a compound of formula (IIIa):

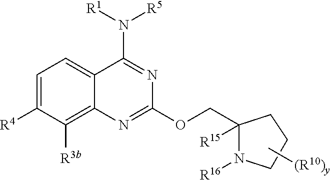

wherein R¹, R^{3b}, R⁴, R⁵, R¹⁰ are as described above for compounds of formula (I); and wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; and y is independently selected from 0, 1, 2, 3, and 4.

In an embodiment, the compound of formula (I) is a compound of formula (IV):

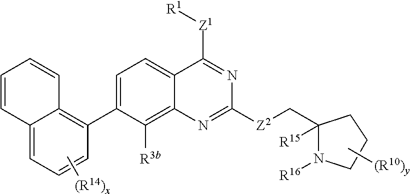

wherein R¹, R^{3b}, R¹⁰, R¹⁴, Z¹ and Z² are as described above for compounds of formula (I); wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; x is independently selected from 0, 1, 2, 3, and 4; and y is independently selected from 0, 1, 2, 3, and 4.

In an embodiment, the compound of formula (I) is a compound of formula (IVa):

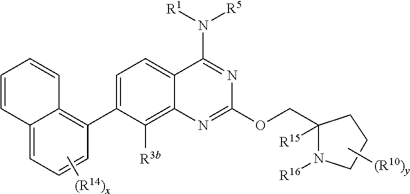

wherein R¹, R^{3b}, R⁵, R¹⁰, R¹⁴ are as described above for compounds of formula (I); wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; x is independently selected from 0, 1, 2, 3, and 4; and y is independently selected from 0, 1, 2, 3, and 4.

In an embodiment, the compound of formula (I) is a compound of formula (V):

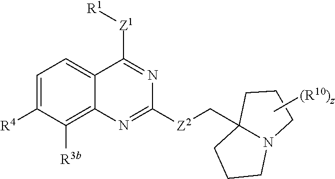

wherein R¹, R^{3b}, R⁴, R¹⁰, Z¹ and Z² are as described above for compounds of formula (I); and wherein z is independently selected from 0, 1, 2, 3, and 4. For the absence of doubt, throughout this specification, the z R¹⁰ groups may be attached to either ring of the pyrrolizidinyl group.

In an embodiment, the compound of formula (I) is a compound of formula (Va):

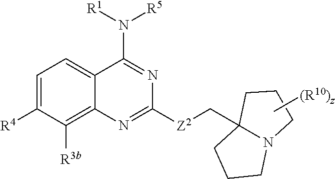

wherein R¹, R^{3b}, R⁴, R⁵, R¹⁰ are as described above for compounds of formula (I); and wherein z is independently selected from 0, 1, 2, 3, and 4. For the absence of doubt, throughout this specification, the z R¹⁰ groups may be attached to either ring of the pyrrolizidinyl group.

In an embodiment, the compound of formula (I) is a compound of formula (VI):

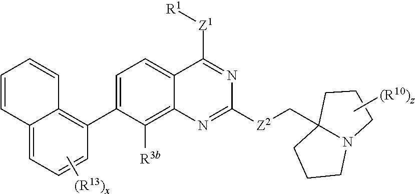

wherein R¹, R^{3b}, R¹⁰, R¹⁴, Z¹ and Z² are as described above for compounds of formula (I); wherein x is independently selected from 0, 1, 2, 3, and 4; and z is independently selected from 0, 1, 2, 3 and 4.

In an embodiment, the compound of formula (I) is a compound of formula (Via):

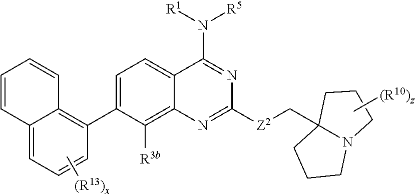

wherein R¹, R^{3b}, R⁵, R¹⁰, R¹⁴ are as described above for compounds of formula (I); wherein x is independently selected from 0, 1, 2, 3, and 4; and z is independently selected from 0, 1, 2, 3 and 4.

In an embodiment, the compound of formula (I) or (Ia) is a compound of formula (VII):

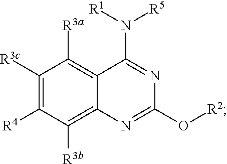

wherein R¹, R², R^{3a}, R^{3c}, R⁴, and R⁵ are as described above for compounds of formula (I) or (Ia).

In an embodiment, the compound of formula (I) or (Ia) is a compound of formula (VIII):

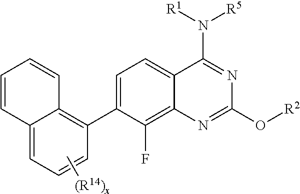

wherein R¹, R², R⁵ and R¹⁴ are as described above for compounds of formula (I) or (Ia); and x is independently selected from 0, 1, 2, 3, and 4. For the absence of doubt, throughout this specification, the x R¹⁴ groups may be attached to either ring of the naphthyl group.

In an embodiment, the compound of formula (I) or (Ia) is a compound of formula (IX):

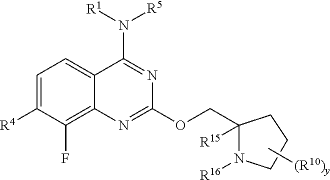

wherein R¹, R⁴, R⁵, R¹⁰ are as described above for compounds of formula (I) or (Ia); and wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; and y is independently selected from 0, 1, 2, 3, and 4.

In an embodiment, the compound of formula (I) or (Ia) is a compound of formula (X):

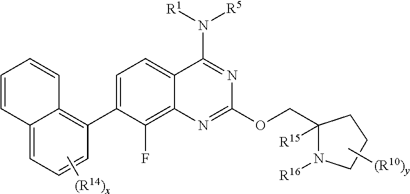

wherein R¹, R¹⁰, R⁵, R¹⁴ are as described above for compounds of formula (I) or (Ia); wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; x is independently selected from 0, 1, 2, 3, and 4; and y is independently selected from 0, 1, 2, 3, and 4.

In an embodiment, the compound of formula (I) or (Ia) is a compound of formula (XI):

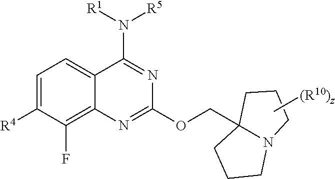

wherein R¹, R⁴, R⁵, R¹⁰ are as described above for compounds of formula (I) or (Ia); and wherein z is independently selected from 0, 1, 2, 3, and 4. For the absence of doubt, throughout this specification, the z R¹⁰ groups may be attached to either ring of the pyrrolizidinyl group.

In an embodiment, the compound of formula (I) or (Ia) is a compound of formula (XII):

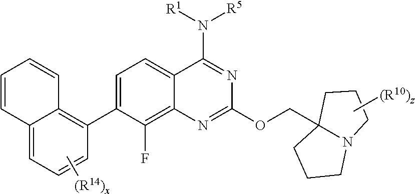

wherein R¹, R⁵, R¹⁰, R¹⁴ are as described above for compounds of formula (I) or (Ia); wherein x is independently selected from 0, 1, 2, 3, and 4; and z is independently selected from 0, 1, 2, 3 and 4.

The following embodiments apply to compounds of any of formulae (I)-(XII). These embodiments are independent and interchangeable. Any one embodiment may be combined with any other embodiment, where chemically allowed. In other words, any of the features described in the following embodiments may (where chemically allowable) be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

Z¹ may be —O—. Z¹ may be —NR⁵—.

Z² may be —O—. Z² may be —NR⁶—.

R¹ is independently C₀-C₃-alkylene-R^{1a} wherein R^{1a} is independently selected from a 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups.

R¹ may be C₀-C₃-alkylene-R^{1a}. R¹ may be C₀-C₃-alkylene-R^{1a} wherein R^{1a} is independently selected from an oxygen containing 4- to 7-membered heterocycloalkyl ring, a 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups. R¹ may be CH₂—R^{1a} wherein R^{1a} is independently selected from a 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups.

R¹ may be C₀-C₃-alkylene-R^{1a} wherein R^{1a} is independently selected from an oxygen containing 4- to 7-membered heterocycloalkyl ring, a nitrogen containing 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups. R¹ may be CH₂—R^{1a} wherein R^{1a} is independently selected from a nitrogen containing 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups.

R¹ may be R^{1a} wherein R^{1a} is independently selected from a 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups.

R¹ may be R^{1a} wherein R^{1a} is independently selected from a nitrogen containing 4-to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups. R¹ may be R^{1a} wherein R^{1a} is an oxygen containing 4- to 7-membered heterocycloalkyl ring; wherein said heterocycloalkyl ring is optionally substituted with from 1 to 4 R⁹ groups. R¹ may be R^{1a} wherein R^{1a} is an oxygen containing 4- to 7-membered heterocycloalkyl ring e.g. a tetrahydropyranyl ring.

R¹ may be C₀-C₃-alkylene-R^{1a} wherein R^{1a} is a 4- to 7-membered heterocycloalkyl ring; wherein said heterocycloalkyl ring is optionally substituted with from 1 to 4 R⁹ groups.

R¹ may be CH₂-alkylene-R^{1a} wherein R^{1a} is a 4- to 7-membered heterocycloalkyl ring; wherein said heterocycloalkyl ring is optionally substituted with from 1 to 4 R⁹ groups.

R¹ may be C₀-C₃-alkylene-R^{1a} wherein R^{1a} is a nitrogen containing 4- to 7-membered heterocycloalkyl ring; wherein said heterocycloalkyl ring is optionally substituted with from 1 to 4 R⁹ groups. R¹ may be CH₂-alkylene-R^{1a} wherein R^{1a} is a nitrogen containing 4- to 7-membered heterocycloalkyl ring; wherein said heterocycloalkyl ring is optionally substituted with from 1 to 4 R⁹ groups.

R¹ may be C₀-C₃-alkylene-R^{1a} wherein R^{1a} is a 4- to 7-membered heterocycloalkyl ring wherein the ring does not comprise any nitrogen atoms; wherein said heterocycloalkyl ring is optionally substituted with from 1 to 4 R⁹ groups. R¹ may be CH₂-alkylene-R^{1a} wherein R^{1a} is a 4- to 7-membered heterocycloalkyl ring wherein the ring does not comprise any nitrogen atoms; wherein said heterocycloalkyl ring is optionally substituted with from 1 to 4 R⁹ groups.

R¹ may be R^{1a} wherein R^{1a} is a 4- to 7-membered heterocycloalkyl ring; wherein said heterocycloalkyl ring is optionally substituted with from 1 to 4 R⁹ groups. R¹ may be R^{1a} wherein R^{1a} is a nitrogen containing 4- to 7-membered heterocycloalkyl ring; wherein said heterocycloalkyl ring is optionally substituted with from 1 to 4 R⁹ groups. R¹ may be R^{1a} wherein R^{1a} is a 4- to 7-membered heterocycloalkyl ring; wherein said heterocycloalkyl ring is optionally substituted with from 1 to 4 R⁹ groups wherein the ring does not comprise any nitrogen atoms.

R¹ may be C₀-C₃-alkylene-R^{1a} wherein R^{1a} is a C₃-C₇-cycloalkyl ring substituted with an NR⁷R⁸ group; wherein said cycloalkyl ring is optionally substituted with from 1 to 4 R⁹ groups. R¹ may be CH₂-alkylene-R^{1a} wherein R^{1a} is a C₃-C₇-cycloalkyl ring substituted with an NR⁷R⁸ group; wherein said cycloalkyl ring is optionally substituted with from 1 to 4 R⁹ groups.

R¹ may be R^{1a} wherein R^{1a} is a C₃-C₇-cycloalkyl ring substituted with an NR⁷R⁸ group; wherein said cycloalkyl ring is optionally substituted with from 1 to 4 R⁹ groups.

R¹ may be C₂-C₈-alkylene-R^{1b}. R¹ may be C₂-C₃-alkylene-R^{1b}. R¹ may be C₃-alkylene-R^{1b}. R^{1b} may be independently selected from: NR⁷R⁸, OR⁸ and SR⁸. R^{1b} may be OR⁸. R^{1b} may be SR⁸. R^{1b} may be NR⁷R⁸. R⁸ may be C₁-C₄-alkyl, e.g. Me.

It may be that R¹ and R⁵ are selected such that NR¹R⁵ comprises no more than a single amine, wherein said single amine may be a primary, secondary or tertiary amine. Compounds having no more than a single amine at this position surprisingly exhibit broad spectrum inhibition at similar concentrations across a range of mutant KRAS forms as well as wild type KRAS rather than inhibition of the specific KRAS G12C and G12D proteins. The compounds of the invention exhibit broad spectrum inhibition at similar concentrations of KRAS mutants including KRAS G12D, KRAS G12C, KRAS G12V, KRAS G12A, KRAS G13D and KRAS Q61H as well as wild-type KRAS. As such these compounds may be of therapeutic benefit in treating cancers bearing KRAS mutations beyond G12D and G12C, as well as cancers dependent on wild type KRAS.

It may be that R¹ and R⁵ together with the nitrogen to which they are attached form a ring system selected from: a monocyclic 4- to 7-membered heterocycloalkyl group, optionally substituted with from 1 to 4 R⁹ groups; a fused or spirofused bicyclic 6- to 11-membered heterocycloalkyl group, optionally substituted with from 1 to 4 R⁹ groups; and a bridged bicyclic 6- to 11-membered heterocycloalkyl group, optionally substituted with from 1 to 4 R⁹ groups; wherein the nitrogen to which R¹ and R⁵ are attached is the only heteroatom in the ring system.

It may be that R¹ and R⁵ together with the nitrogen to which they are attached form a ring system selected from: a monocyclic 4- to 7-membered heterocycloalkyl group, optionally substituted with from 1 to 4 R⁹ groups; a fused or spirofused bicyclic 6- to 11-membered heterocycloalkyl group, optionally substituted with from 1 to 4 R⁹ groups; and a bridged bicyclic 6- to 11-membered heterocycloalkyl group, optionally substituted with from 1 to 4 R⁹ groups; wherein the nitrogen to which R¹ and R⁵ are attached is the only nitrogen in the ring system.

It may be that R¹ and R⁵ are selected such that the nitrogen of NR¹R⁵ is the nitrogen of the single amine. It may be that R¹ and R⁵ are selected such that NR¹R⁵ is the single amine. For the avoidance of doubt, the term “amine” as used herein encompasses primary amines, e.g., methylamine; secondary amines, e.g., dimethylamine; tertiary amines, e.g., trimethylamine; cyclic amines, e.g., piperidine. For the avoidance of doubt, the term “amine” as used herein excludes amides and lactams, e.g., piperazinonyl.

It may be that R¹ and R⁵ together with the nitrogen to which they are attached form a ring system having the structure:

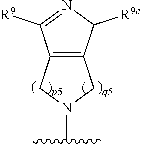

wherein R^{9c} is selected from H and C₁-C₄-alkyl, p5 and q5 and are each selected from 0, 1, 2 and 3; providing that the sum of p5 and q5 is 1 or greater.

It may be that R¹ and R⁵ together with the nitrogen to which they are attached form a ring system having a structure selected from:

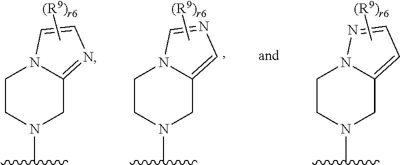

wherein r6 is selected from 0, 1 and 2.

It may be that R¹ and R⁵ together with the nitrogen to which they are attached form a ring system selected from: a monocyclic 4- to 7-membered group heterocycloalkyl group, optionally substituted with from 1 to 4 R⁹ groups; a fused, spirofused or bridged bicyclic 6- to 11-membered heterocycloalkyl group, optionally substituted with from 1 to 4 R⁹ groups.

It may be that R¹ and R⁵ together with the nitrogen to which they are attached form a ring system selected from: a monocyclic 4- to 7-membered group heterocycloalkyl group, optionally substituted with from 1 to 4 R⁹ groups; a fused, spirofused or bridged bicyclic 6- to 11-membered heterocycloalkyl group, optionally substituted with from 1 to 4 R⁹ groups; wherein the ring system does not comprise a nitrogen other than the nitrogen to which R¹ and R⁵ are attached.

It may be that R¹ and R⁵ together with the nitrogen to which they are attached form a ring system selected from: a monocyclic 4- to 7-membered group heterocycloalkyl group, optionally substituted with from 1 to 4 R⁹ groups; a fused or spirofused bicyclic 6- to 11-membered heterocycloalkyl group, optionally substituted with from 1 to 4 R⁹ groups; and a bridged bicyclic 6- to 11-membered heterocycloalkyl group, optionally substituted with from 1 to 4 R⁹ groups.

It may be that R¹ and R⁵ together with the nitrogen to which they are attached form a ring system selected from: a monocyclic 4- to 7-membered group heterocycloalkyl group, optionally substituted with from 1 to 4 R⁹ groups; a fused or spirofused bicyclic 6- to 11-membered heterocycloalkyl group, optionally substituted with from 1 to 4 R⁹ groups.

It may be that R¹ and R⁵ together with the nitrogen to which they are attached form a 6 or 7-membered group heterocycloalkyl group, optionally substituted with from 1 to 4 R⁹ groups. It may be that R¹ and R⁵ together with the nitrogen to which they are attached form a 6 or 7-membered group heterocycloalkyl group, optionally substituted with from 1 to 4 R⁹ groups, wherein the total number of heteroatoms in the 6 or 7-membered group heterocycloalkyl group is 1 or 2. The total number of heteroatoms may be 2. It may be that R¹ and R⁵ together with the nitrogen to which they are attached form a 6 or 7-membered group heterocycloalkyl group, optionally substituted with 1 R⁹ group.

It may be that R¹ and R⁵ together with the nitrogen to which they are attached form a monocyclic 4- to 7-membered group heterocycloalkyl group, optionally substituted with from 1 to 4 R⁹ groups. It may be that R¹ and R⁵ together with the nitrogen to which they are attached form an unsubstituted monocyclic 4- to 7-membered group heterocycloalkyl group. It may be that there is at least one R⁹ group and that at least one of said R⁹ groups is selected from NR¹²R¹³ and C₁-C₄-alkyl substituted with NR¹²R¹³. It may be that R¹ and R⁵ together with the nitrogen to which they are attached form a ring system having the structure:

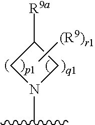

wherein R^{9a} is selected from NR¹²R¹³ and C₁-C₄-alkyl substituted with NR¹²R¹³; p1 is selected from 0, 1, 2 and 3, q1 is selected from 0, 1 and 2; and r1 is selected from 0, 1, 2 and 3. r1 may be 0. R⁹ may independently at each occurrence be methyl. R^{9a} may be selected from NHR¹² and C₁-C₄-alkyl substituted with NHR¹².

It may be that R¹ and R⁵ together with the nitrogen to which they are attached form a monocyclic 4- to 7-membered group heterocycloalkyl group comprising two nitrogen atoms in the ring, optionally substituted with from 1 to 4 R⁹ groups.

It may be that R¹ and R⁵ together with the nitrogen to which they are attached form a ring system having the structure:

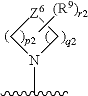

wherein Z⁶ is independently selected from C(O)NR^{9b}, NR^{9b}, O, S, S(O)₂, S(O), S(O)(NR^{9b}) and S(O)(NH); R^{9b} is selected from H and C₁-C₄-alkyl; p2 is selected from 2 and 3, q2 is 2; and r2 is selected from 0, 1, 2 and 3. Z⁶ may be selected from NR^{9b}, O, S, S(O)₂, S(O) and S(O)(NH). Z⁶ may be selected from C(O)NR^{9b}, O, S, S(O)₂, S(O), S(O)(NR^{9b}) and S(O)(NH). Z⁶ may be selected from O, S, S(O)₂, S(O) and S(O)(NH).
Z⁶ may be selected from NR^{9b}, O and S. Z⁶ may be selected from O and S. Z⁶ may be O.

It may be that R¹ and R⁵ together with the nitrogen to which they are attached form a ring system having the structure:

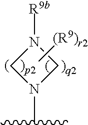

wherein R^{9b} is selected from H and C₁-C₄-alkyl; p2 is selected from 2 and 3, q2 is 2; and r2 is selected from 0, 1, 2 and 3. r2 may be 0. R⁹ may independently at each occurrence be methyl. R^{9b} may be H. R^{9b} may be C₁-C₄-alkyl.

It may be that R¹ and R⁵ together with the nitrogen to which they are attached form a fused or spirofused bicyclic 6- to 11-membered heterocycloalkyl group, optionally substituted with from 1 to 4 R⁹ groups. It may be that R¹ and R⁵ together with the nitrogen to which they are attached form a fused or spirofused bicyclic 6- to 11-membered heterocycloalkyl group comprising two nitrogen atoms in the ring system, optionally substituted with from 1 to 4 R⁹ groups.

It may be that R¹ and R⁵ together with the nitrogen to which they are attached form a spirofused bicyclic 6- to 11-membered heterocycloalkyl group, optionally substituted with from 1 to 4 R⁹ groups. It may be that R¹ and R⁵ together with the nitrogen to which they are attached form a spirofused bicyclic 6- to 11-membered heterocycloalkyl group comprising two nitrogen atoms in the ring system, optionally substituted with from 1 to 4 R⁹ groups. It may be that R¹ and R⁵ together with the nitrogen to which they are attached form a ring system having the structure:

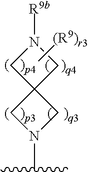

wherein R^{9b} is selected from H and C₁-C₄-alkyl; p3, p4, q3 and q4 are each independently selected from 0, 1, 2 and 3; providing that the sum of p3, p4, q3 and q4 is from 3 to 8, the sum of p3 and q3 is 2 or greater, and the sum of p4 and q4 is 2 or greater; and r3 is selected from 0, 1, 2 and 3. For the absence of doubt, throughout this specification, the r3 R⁹ groups may be attached to either ring of the spirofused bicyclic ring system. r3 may be 0. R⁹ may independently at each occurrence be methyl. R^{9b} may be H.

It may be that R¹ and R⁵ together with the nitrogen to which they are attached form a fused bicyclic 6- to 11-membered heterocycloalkyl group, optionally substituted with from 1 to 4 R⁹ groups. It may be that R¹ and R⁵ together with the nitrogen to which they are attached form a fused bicyclic 6- to 11-membered heterocycloalkyl group comprising two nitrogen atoms in the ring system, optionally substituted with from 1 to 4 R⁹ groups. It may be that R¹ and R⁵ together with the nitrogen to which they are attached form a ring system having the structure:

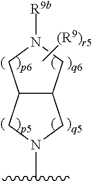

wherein R^{9b} is selected from H and C₁-C₄-alkyl; p5, p6, q5 and are each selected from 0, 1, 2 and 3; providing that the sum of p3, p4, q3 and q4 is from 2 to 7, the sum of p5 and q5 is 1 or greater, and the sum of p6 and q6 is 1 or greater; and r5 is selected from 0, 1, 2 and 3. For the absence of doubt, throughout this specification, the r5 R⁹ groups may be attached to either ring of the fused bicyclic ring system. r5 may be 0. R⁹ may independently at each occurrence be methyl. R^{9b} may be H.

It may be that R¹ and R⁵ together with the nitrogen to which they are attached form a bridged bicyclic 6- to 11-membered heterocycloalkyl group, optionally substituted with from 1 to 4 R⁹ groups.

It may be that R¹ and R⁵ together with the nitrogen to which they are attached form a bridged bicyclic 6- to 11-membered heterocycloalkyl group, optionally substituted with from 1 to 4 R⁹ groups.

It may be that R¹ and R⁵ together with the nitrogen to which they are attached form a ring system having the structure:

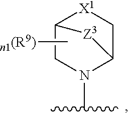

wherein X¹ is independently selected from C(O)NR^{9d}, O and NR¹⁷; Z³ is independently selected from: CH₂, CH₂CH₂, CH₂—O—CH₂CH₂, CH₂—O—CH₂, CH₂—NR¹⁷—CH₂CH₂ and CH₂—NR¹⁷—CH₂; R¹⁷ is independently at each occurrence selected from H, C₁-C₄-haloalkyl, and C₁-C₄-alkyl; R^{9d} is independently selected from H and C₁-C₄-alkyl; and n1 is an integer selected from 0, 1, 2, 3 and 4. For the absence of doubt, throughout this specification, the n1 R⁹ groups may be attached to either ring of the bridged bicyclic ring system. Z³ may be independently selected from: CH₂, CH₂CH₂, CH₂—O—CH₂CH₂, CH₂—O—CH₂, X¹ may be independently selected from O and NR¹⁷. X¹ may be NR¹⁷. X¹ may be NH. n1 may be 0. R⁹ may independently at each occurrence be methyl.

It may be that R¹ and R⁵ together with the nitrogen to which they are attached form a ring system having the structure:

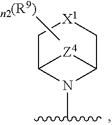

wherein X¹ is independently selected from C(O)NR^{9d}, O and NR¹⁷; Z⁴ is independently selected from: CH₂, CH₂CH₂, CH₂—O—CH₂CH₂, CH₂—O—CH₂, CH₂—NR¹⁷—CH₂CH₂ and CH₂—NR¹⁷—CH₂; R¹⁷ is independently at each occurrence selected from H, C₁-C₄-haloalkyl, and C₁-C₄-alkyl; R^{9d} is independently selected from H and C₁-C₄-alkyl; and n2 is an integer selected from 0, 1, 2, 3 and 4. For the absence of doubt, throughout this specification, the n2 R⁹ groups may be attached to either ring of the bridged bicyclic ring system. Z⁴ may be independently selected from: CH₂, CH₂CH₂, CH₂—O—CH₂CH₂, CH₂—O—CH₂, X¹ may be independently selected from O and NR¹⁷. X¹ may be NR¹⁷. X¹ may be NH. n2 may be 0. R⁹ may independently at each occurrence be methyl.

It may be that R¹ and R⁵ together with the nitrogen to which they are attached form a ring system having the structure:

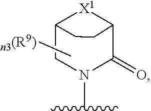

wherein X¹ is independently selected from C(O)NR^{9d}, O and NR¹⁷; Z⁴ is independently selected from: CH₂, CH₂CH₂, CH₂—O—CH₂CH₂, CH₂—O—CH₂, CH₂—NR¹⁷—CH₂CH₂ and CH₂—NR¹⁷—CH₂; R¹⁷ is independently at each occurrence selected from H, C₁-C₄-haloalkyl, and C₁-C₄-alkyl; R^{9d} is independently selected from H and C₁-C₄-alkyl; and n3 is an integer selected from 0, 1, 2, 3 and 4. For the absence of doubt, throughout this specification, the n3 R⁹ groups may be attached to either ring of the bridged bicyclic ring system. X¹ may be independently selected from O and NR¹⁷. X¹ may be NR¹⁷. X¹ may be NH. n3 may be 0. R⁹ may independently at each occurrence be methyl.

It may be that R¹ and R⁵ together with the nitrogen to which they are attached form a ring system having the structure:

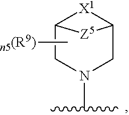

wherein X¹ is independently selected from C(O)NR^{9d}, O and NR¹⁷; Z⁵ is independently selected from: CH₂, CH₂CH₂, CH₂—O—CH₂CH₂, CH₂—O—CH₂, CH₂—NR¹⁷—CH₂CH₂ and CH₂—NR¹⁷—CH₂; R¹⁷ is independently at each occurrence selected from H, C₁-C₄-haloalkyl, and C₁-C₄-alkyl; R^{9d} is independently selected from H and C₁-C₄-alkyl; and n5 is an integer selected from 0, 1, 2, 3 and 4. For the absence of doubt, throughout this specification, the n5 R⁹ groups may be attached to either ring of the bridged bicyclic ring system. Z⁵ is independently selected from: CH₂, CH₂CH₂, CH₂—O—CH₂CH₂, CH₂—O—CH₂. X¹ may be independently selected from O and NR¹⁷. X¹ may be NR¹⁷. X¹ may be NH. n5 may be 0. R⁹ may independently at each occurrence be methyl.

It may be that R¹ and R⁵ together with the nitrogen to which they are attached form a ring system having the structure:

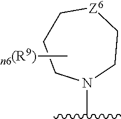

wherein Z⁶ is independently selected from C(O)NR^{9b}, O, S, S(O)₂, S(O), S(O)(NR^{9b}), S(O)(NH) and NR^{9b}; R^{9b} is independently at each occurrence selected from H and C₁-C₄-alkyl; and n6 is an integer selected from 0, 1, 2, 3 and 4. Z⁶ may be selected from NR^{9b}, O, S, S(O)₂, S(O), S(O)(NR^{9b}), and S(O)(NH). Z⁶ may be selected from C(O)NR^{9b}, O, S, S(O)₂, S(O) and S(O)(NH). Z⁶ may be selected from O, S, S(O)₂, S(O) and S(O)(NH). Z⁶ may be selected from NR^{9b}, O and S. Z⁶ may be selected from O and S. Z⁶ may be O.

It may be that R¹ and R⁵ together with the nitrogen to which they are attached form a ring system having the structure:

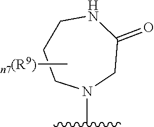

wherein n7 is an integer selected from 0, 1, 2 and 3.

It may be that R¹ and R⁵ together with the nitrogen to which they are attached form a ring system having the structure:

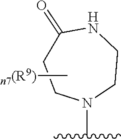

wherein n7 is an integer selected from 0, 1, 2 and 3.

It may be that R¹ and R⁵ together with the nitrogen to which they are attached form a ring system having the structure:

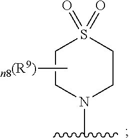

wherein n8 is an integer selected from 0, 1, 2 and 3.
n7 may be 0.
R² may be C₀-C₄-alkylene-R^{2a}. R² may be CH₂—R^{2a}. R^{2a} may be selected from monocyclic 4- to 7-membered heterocycloalkyl group, a fused, spirofused or bridged bicyclic 6- to 11-membered heterocycloalkyl group; wherein said R^{2a} group is optionally substituted with from 1 to 6 R¹⁰ groups. R^{2a} may comprise at least one nitrogen in the ring system. R^{2a} may comprise a single nitrogen in the ring system. R^{2a} may be selected from monocyclic 4-to 7-membered heterocycloalkyl group, a fused, spirofused or bridged bicyclic 6- to 11-membered heterocycloalkyl group; wherein said R^{2a} group is optionally substituted with from 1 to 6 R¹⁰ groups and wherein R^{2a} comprises at least one nitrogen in the ring system. R^{2a} may be monocyclic 4- to 7-membered heterocycloalkyl group; wherein said R^{2a} group is optionally substituted with from 1 to 6 R¹⁰ groups and wherein R^{2a} comprises at least one nitrogen in the ring system. R^{2a} may be a fused, spirofused or bridged bicyclic 6- to 11-membered heterocycloalkyl group; wherein said R^{2a} group is optionally substituted with from 1 to 6 R¹⁰ groups and wherein R^{2a} comprises at least one nitrogen in the ring system.
R² may have the structure:

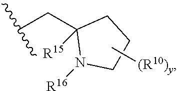

wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; and y is independently selected from 0, 1, 2, 3, and 4. y may be selected from 0 and 1. y may be 0. y may be 1. R¹⁵ may be H. R¹⁶ may be C₁-C₄-alkyl.
R² may have the structure:

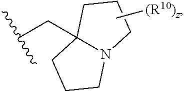

wherein z is independently selected from 0, 1, 2, 3, and 4. z may be selected from 0 and 1. z may be 0. z may be 1.
R² may have the structure:

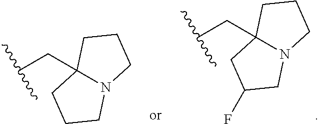 [SMILES: *CC12CCCN1CCC2 ; *CC12CCCN1CC(F)C2]

R^{3a} may be H.
R^{3b} may be selected from halo, C₁-C₄-alkyl, O—C₁-C₄-alkyl, C₁-C₄-haloalkyl, O—C₁-C₄-haloalkyl, cyclopropyl, nitro and cyano. R^{3b} may be F. R^{3b} may be C₁-C₄-alkyl, e.g. Me.
R^{3c} may be selected from halo, C₁-C₄-alkyl, O—C₁-C₄-alkyl, C₁-C₄-haloalkyl, O—C₁-C₄-haloalkyl, cyclopropyl, nitro and cyano. R^{3c} may be F. R^{3c} may be C₁-C₄-alkyl, e.g. Me. R^{3c} may be H.

It may be that both R^{3a} and R^{3c} are H·R⁴ may be phenyl, said phenyl being optionally fused to a C₅-C₇-cycloalkyl ring, wherein R⁴ is optionally substituted with from 1 to 4 R¹⁴ groups. R⁴ may be phenyl, optionally substituted with from 1 to 4 R¹⁴ groups.

R⁴ may have the structure:

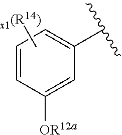

wherein R^{12a} is independently H or C₁-C₄-alkyl; x1 is independently selected from 0, 1, 2 and 3. R^{12a} may be H.

R⁴ may be naphthyl, optionally substituted with from 1 to 4 R¹⁴ groups. R⁴ may have the structure:

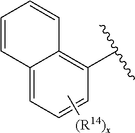

wherein x is independently selected from 0, 1, 2, 3, and 4. For the absence of doubt, throughout this specification, the x R¹⁴ groups may be attached to either ring of the naphthyl group.

R⁴ may have the structure:

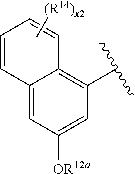

wherein R^{12a} is independently H or C₁-C₄-alkyl; x2 is independently selected from 0, 1, 2 and 3. For the absence of doubt, throughout this specification, the x2 R¹⁴ groups may be attached to either ring of the naphthyl group. R^{12a} may be H.

R⁴ may have the structure:

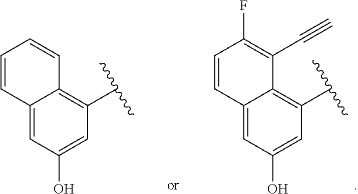 [SMILES: *c1cc(O)cc2ccccc12 ; *c1cc(O)cc2ccc(F)c(C#C)c12]

R⁴ may be 5-, 6-, 9- or 10-membered monocyclic or bicyclic heteroaryl, optionally substituted with from 1 to 4 R¹⁴ groups. R⁴ may be 9- or 10-membered bicyclic heteroaryl, optionally substituted with from 1 to 4 R¹⁴ groups.

R⁵ may be H. R⁵ may be C₁-C₄-alkyl, e.g. methyl.

R⁶ may be H. R⁶ may be C₁-C₄-alkyl, e.g. methyl.

R⁷ may be selected from H and C₁-C₄-alkyl. R⁷ may be H. R⁷ may be C₁-C₄-alkyl, e.g. methyl.

R⁸ may be selected from H and C₁-C₄-alkyl. R⁸ may be H. R⁸ may be C₁-C₄-alkyl, e.g. methyl.

R⁹ may be independently at each occurrence selected from oxo, fluoro, cyano, NR¹²R¹³, OR¹², C₁-C₄-alkyl, C₁-C₄-alkyl substituted with NR¹²R¹³, C₁-C₄-alkyl substituted with OR¹², C₁-C₄-alkyl substituted with cyano. R⁹ may be independently at each occurrence selected from oxo, fluoro, NR¹²R¹³, OR¹², C₁-C₄-alkyl, C₁-C₄-alkyl substituted with NR¹²R¹³ and C₁-C₄-alkyl substituted with OR¹².

R⁹ may be independently at each occurrence selected from oxo, halo, cyano, NR¹²R¹³ provided that R¹² is not H and R¹³ is not H, OR¹², CO₂R¹², CONR¹²R¹², C₁-C₄-alkyl, C₁-C₄-alkyl substituted with NR¹²R¹³ provided that R¹² is not H and R¹³ is not H, C₁-C₄-alkyl substituted with OR¹², C₁-C₄-alkyl substituted with cyano, C₂-C₄-alkenyl, C₂-C₄-alkynyl, C₁-C₄-haloalkyl and cyclopropyl.

R¹⁰ may be independently at each occurrence selected from oxo, halo, cyano, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₁-C₄-alkyl substituted with cyano, C₂-C₄-alkenyl, C₂-C₄-alkynyl, C₁-C₄-haloalkyl and cyclopropyl.

R¹⁰ may be independently at each occurrence selected from oxo, fluoro, NR¹²R¹³, OR¹², C₁-C₄-alkyl, C₁-C₄-alkyl substituted with NR¹²R¹³ and C₁-C₄-alkyl substituted with OR¹².

R¹¹ may be each independently at each occurrence selected from halo, cyano, nitro, NR¹²R¹³, OR¹², C₁-C₄-alkyl, C₁-C₄-alkyl substituted with NR¹²R¹³, C₁-C₄-alkyl substituted with OR¹², C₁-C₄-haloalkyl and cyclopropyl. R¹¹ may be each independently at each occurrence selected from OR¹², C₁-C₄-alkyl, C₁-C₄-haloalkyl and cyclopropyl.

R¹² may independently at each occurrence be selected from H and C₁-C₄-alkyl.

R¹³ may independently at each occurrence be selected from H and C₁-C₄-alkyl.

R¹⁴ may be each independently at each occurrence selected from halo, cyano, nitro, NR¹²R¹³, OR¹², C₁-C₄-alkyl, C₁-C₄-alkyl substituted with NR¹²R¹³, C₁-C₄-alkyl substituted with OR¹², C₁-C₄-haloalkyl and cyclopropyl. R¹⁴ may be each independently at each occurrence selected from OR¹², C₁-C₄-alkyl, C₁-C₄-haloalkyl and cyclopropyl.

The compound of formula (I) may be selected from:

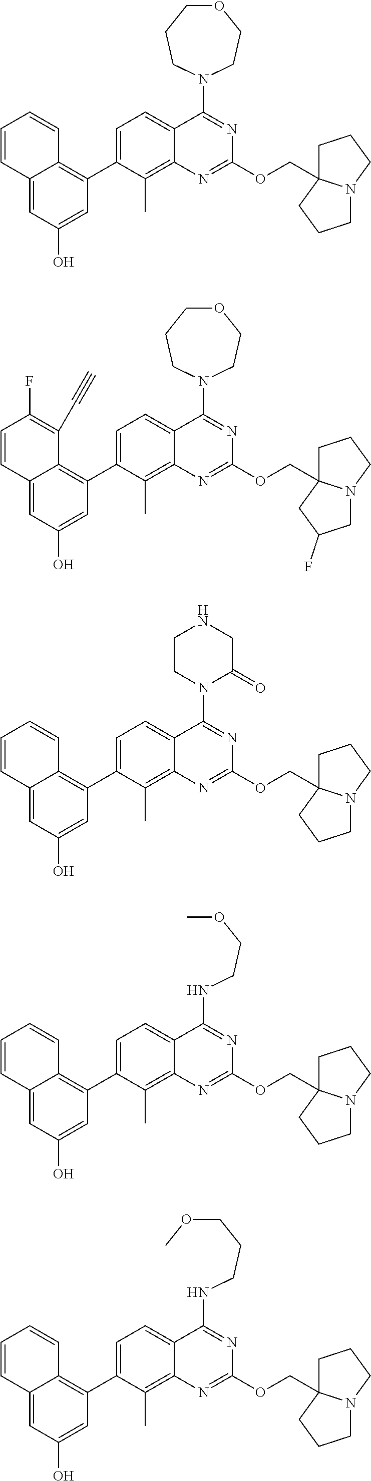 [SMILES: Cc1c(-c2cc(O)cc3ccccc23)ccc2c(N3CCCOCC3)nc(OCC34CCCN3CCC4)nc12 ; C#Cc1c(F)ccc2cc(O)cc(-c3ccc4c(N5CCCOCC5)nc(OCC56CCCN5CC(F)C6)nc4c3C)c12 ; Cc1c(-c2cc(O)cc3ccccc23)ccc2c(N3CCNCC3=O)nc(OCC34CCCN3CCC4)nc12 ; COCCNc1nc(OCC23CCCN2CCC3)nc2c(C)c(-c3cc(O)cc4ccccc34)ccc12 ; COCCCNc1nc(OCC23CCCN2CCC3)nc2c(C)c(-c3cc(O)cc4ccccc34)ccc12]

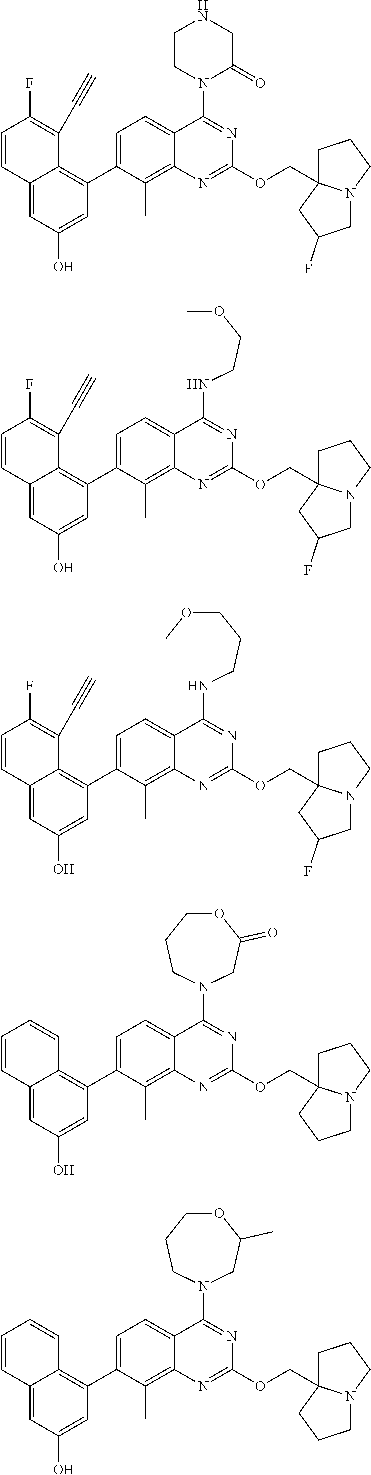 [SMILES: C#Cc1c(F)ccc2cc(O)cc(-c3ccc4c(N5CCNCC5=O)nc(OCC56CCCN5CC(F)C6)nc4c3C)c12 ; C#Cc1c(F)ccc2cc(O)cc(-c3ccc4c(NCCOC)nc(OCC56CCCN5CC(F)C6)nc4c3C)c12 ; C#Cc1c(F)ccc2cc(O)cc(-c3ccc4c(NCCCOC)nc(OCC56CCCN5CC(F)C6)nc4c3C)c12 ; Cc1c(-c2cc(O)cc3ccccc23)ccc2c(N3CCCOC(=O)C3)nc(OCC34CCCN3CCC4)nc12 ; Cc1c(-c2cc(O)cc3ccccc23)ccc2c(N3CCCOC(C)C3)nc(OCC34CCCN3CCC4)nc12]

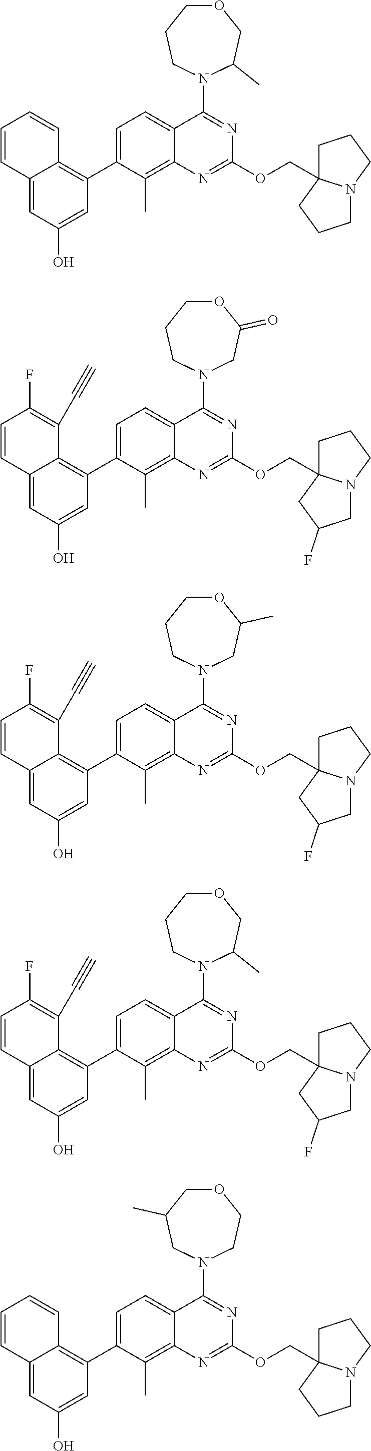 [SMILES: Cc1c(-c2cc(O)cc3ccccc23)ccc2c(N3CCCOCC3C)nc(OCC34CCCN3CCC4)nc12 ; C#Cc1c(F)ccc2cc(O)cc(-c3ccc4c(N5CCCOC(=O)C5)nc(OCC56CCCN5CC(F)C6)nc4c3C)c12 ; C#Cc1c(F)ccc2cc(O)cc(-c3ccc4c(N5CCCOC(C)C5)nc(OCC56CCCN5CC(F)C6)nc4c3C)c12 ; C#Cc1c(F)ccc2cc(O)cc(-c3ccc4c(N5CCCOCC5C)nc(OCC56CCCN5CC(F)C6)nc4c3C)c12 ; Cc1c(-c2cc(O)cc3ccccc23)ccc2c(N3CCOCC(C)C3)nc(OCC34CCCN3CCC4)nc12]

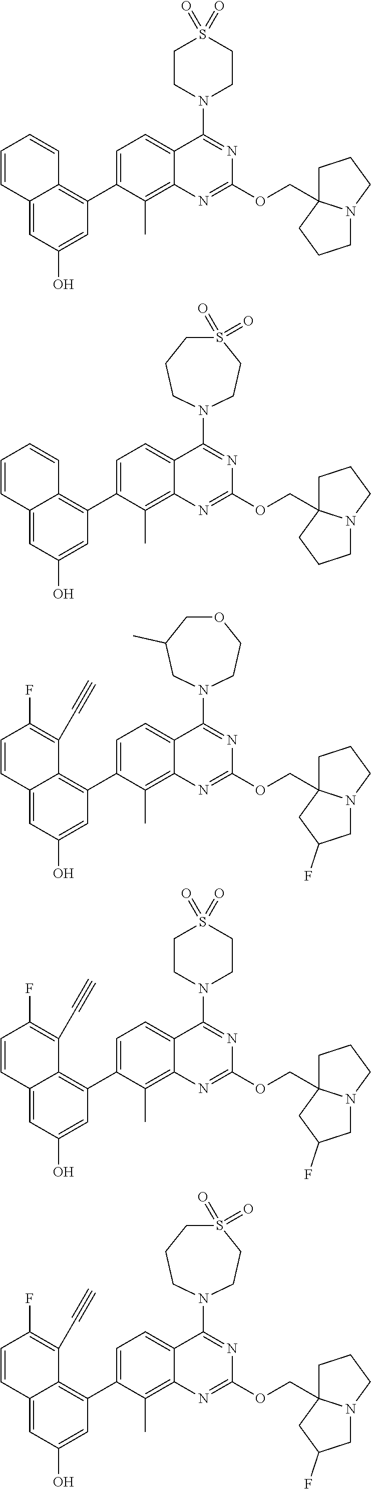 [SMILES: Cc1c(-c2cc(O)cc3ccccc23)ccc2c(N3CCS(=O)(=O)CC3)nc(OCC34CCCN3CCC4)nc12 ; Cc1c(-c2cc(O)cc3ccccc23)ccc2c(N3CCCS(=O)(=O)CC3)nc(OCC34CCCN3CCC4)nc12 ; C#Cc1c(F)ccc2cc(O)cc(-c3ccc4c(N5CCOCC(C)C5)nc(OCC56CCCN5CC(F)C6)nc4c3C)c12 ; C#Cc1c(F)ccc2cc(O)cc(-c3ccc4c(N5CCS(=O)(=O)CC5)nc(OCC56CCCN5CC(F)C6)nc4c3C)c12 ; C#Cc1c(F)ccc2cc(O)cc(-c3ccc4c(N5CCCS(=O)(=O)CC5)nc(OCC56CCCN5CC(F)C6)nc4c3C)c12]

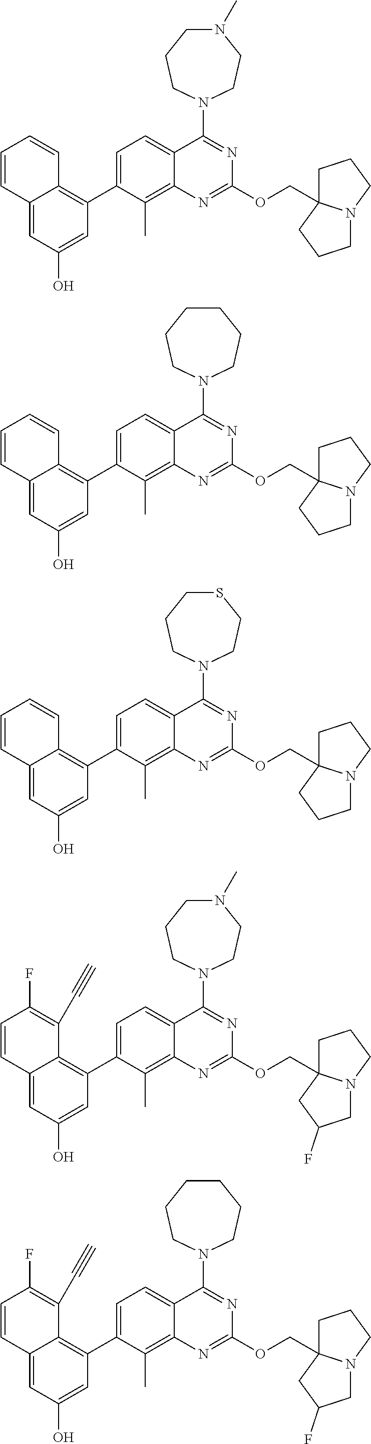 [SMILES: Cc1c(-c2cc(O)cc3ccccc23)ccc2c(N3CCCN(C)CC3)nc(OCC34CCCN3CCC4)nc12 ; Cc1c(-c2cc(O)cc3ccccc23)ccc2c(N3CCCCCC3)nc(OCC34CCCN3CCC4)nc12 ; Cc1c(-c2cc(O)cc3ccccc23)ccc2c(N3CCCSCC3)nc(OCC34CCCN3CCC4)nc12 ; C#Cc1c(F)ccc2cc(O)cc(-c3ccc4c(N5CCCN(C)CC5)nc(OCC56CCCN5CC(F)C6)nc4c3C)c12 ; C#Cc1c(F)ccc2cc(O)cc(-c3ccc4c(N5CCCCCC5)nc(OCC56CCCN5CC(F)C6)nc4c3C)c12]

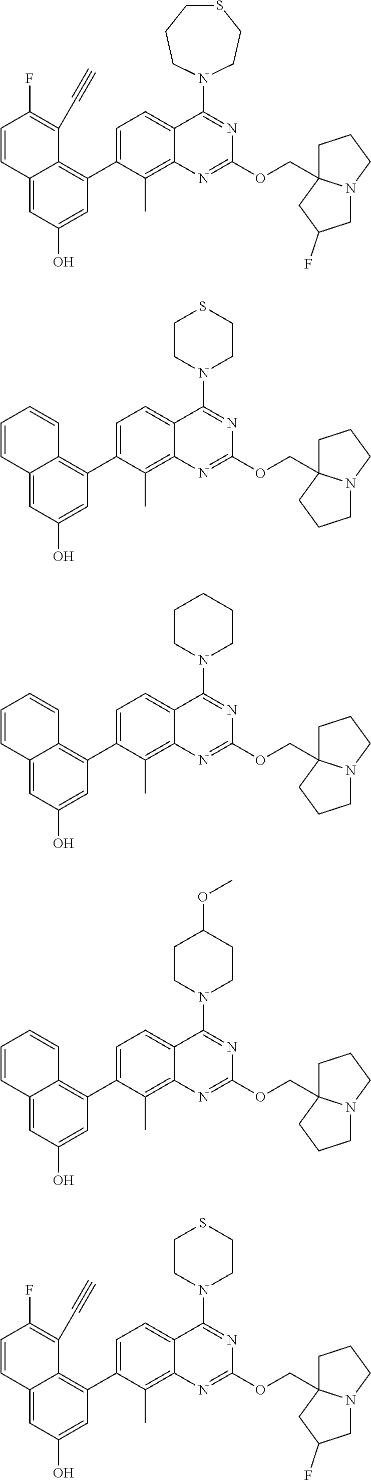 [SMILES: C#Cc1c(F)ccc2cc(O)cc(-c3ccc4c(N5CCCSCC5)nc(OCC56CCCN5CC(F)C6)nc4c3C)c12 ; Cc1c(-c2cc(O)cc3ccccc23)ccc2c(N3CCSCC3)nc(OCC34CCCN3CCC4)nc12 ; Cc1c(-c2cc(O)cc3ccccc23)ccc2c(N3CCCCC3)nc(OCC34CCCN3CCC4)nc12 ; COC1CCN(c2nc(OCC34CCCN3CCC4)nc3c(C)c(-c4cc(O)cc5ccccc45)ccc23)CC1 ; C#Cc1c(F)ccc2cc(O)cc(-c3ccc4c(N5CCSCC5)nc(OCC56CCCN5CC(F)C6)nc4c3C)c12]

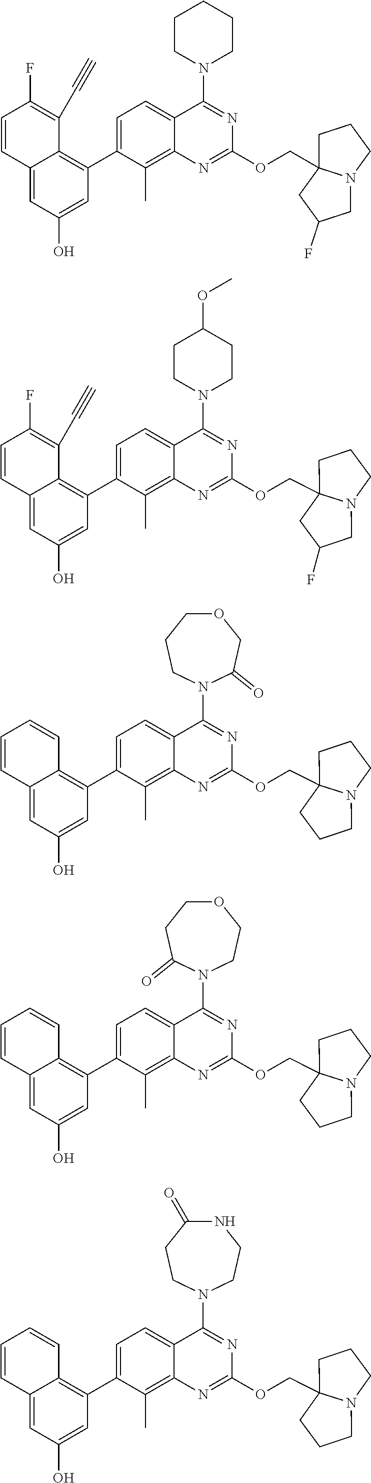 [SMILES: C#Cc1c(F)ccc2cc(O)cc(-c3ccc4c(N5CCCCC5)nc(OCC56CCCN5CC(F)C6)nc4c3C)c12 ; C#Cc1c(F)ccc2cc(O)cc(-c3ccc4c(N5CCC(OC)CC5)nc(OCC56CCCN5CC(F)C6)nc4c3C)c12 ; Cc1c(-c2cc(O)cc3ccccc23)ccc2c(N3CCCOCC3=O)nc(OCC34CCCN3CCC4)nc12 ; Cc1c(-c2cc(O)cc3ccccc23)ccc2c(N3CCOCCC3=O)nc(OCC34CCCN3CCC4)nc12 ; Cc1c(-c2cc(O)cc3ccccc23)ccc2c(N3CCNC(=O)CC3)nc(OCC34CCCN3CCC4)nc12]

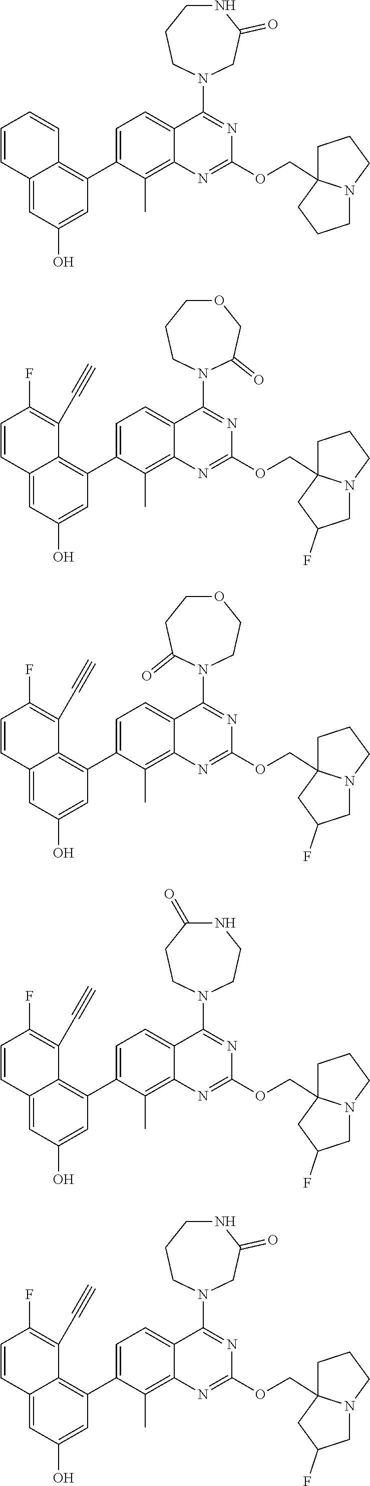 [SMILES: Cc1c(-c2cc(O)cc3ccccc23)ccc2c(N3CCCNC(=O)C3)nc(OCC34CCCN3CCC4)nc12 ; C#Cc1c(F)ccc2cc(O)cc(-c3ccc4c(N5CCCOCC5=O)nc(OCC56CCCN5CC(F)C6)nc4c3C)c12 ; C#Cc1c(F)ccc2cc(O)cc(-c3ccc4c(N5CCOCCC5=O)nc(OCC56CCCN5CC(F)C6)nc4c3C)c12 ; C#Cc1c(F)ccc2cc(O)cc(-c3ccc4c(N5CCNC(=O)CC5)nc(OCC56CCCN5CC(F)C6)nc4c3C)c12 ; C#Cc1c(F)ccc2cc(O)cc(-c3ccc4c(N5CCCNC(=O)C5)nc(OCC56CCCN5CC(F)C6)nc4c3C)c12]

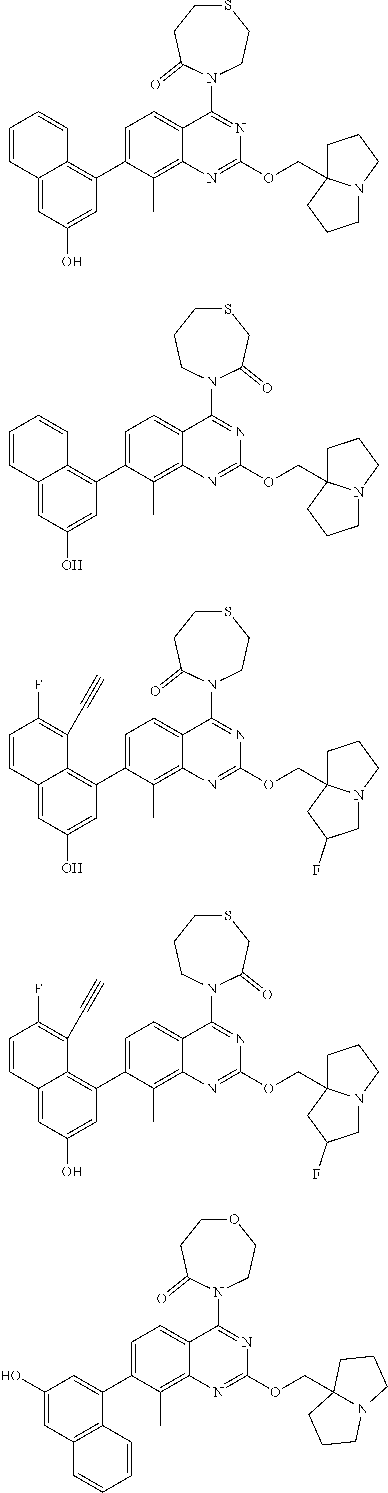 [SMILES: Cc1c(-c2cc(O)cc3ccccc23)ccc2c(N3CCSCCC3=O)nc(OCC34CCCN3CCC4)nc12 ; Cc1c(-c2cc(O)cc3ccccc23)ccc2c(N3CCCSCC3=O)nc(OCC34CCCN3CCC4)nc12 ; C#Cc1c(F)ccc2cc(O)cc(-c3ccc4c(N5CCSCCC5=O)nc(OCC56CCCN5CC(F)C6)nc4c3C)c12 ; C#Cc1c(F)ccc2cc(O)cc(-c3ccc4c(N5CCCSCC5=O)nc(OCC56CCCN5CC(F)C6)nc4c3C)c12 ; Cc1c(-c2cc(O)cc3ccccc23)ccc2c(N3CCOCCC3=O)nc(OCC34CCCN3CCC4)nc12]

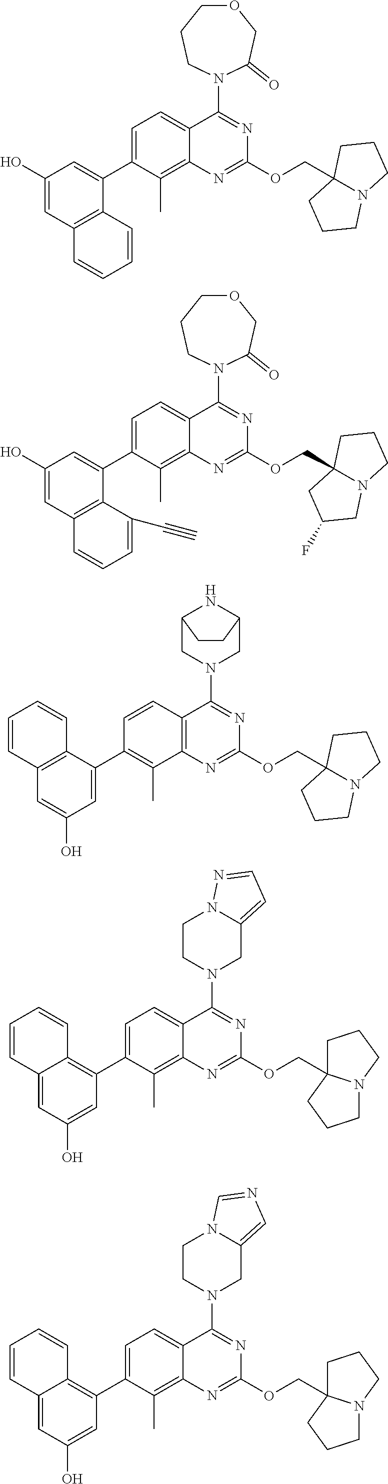 [SMILES: Cc1c(-c2cc(O)cc3ccccc23)ccc2c(N3CCCOCC3=O)nc(OCC34CCCN3CCC4)nc12 ; C#Cc1cccc2cc(O)cc(-c3ccc4c(N5CCCOCC5=O)nc(OC[C@@]56CCCN5C[C@H](F)C6)nc4c3C)c12 ; Cc1c(-c2cc(O)cc3ccccc23)ccc2c(N3CC4CCC(C3)N4)nc(OCC34CCCN3CCC4)nc12 ; Cc1c(-c2cc(O)cc3ccccc23)ccc2c(N3CCn4nccc4C3)nc(OCC34CCCN3CCC4)nc12 ; Cc1c(-c2cc(O)cc3ccccc23)ccc2c(N3CCn4cncc4C3)nc(OCC34CCCN3CCC4)nc12]

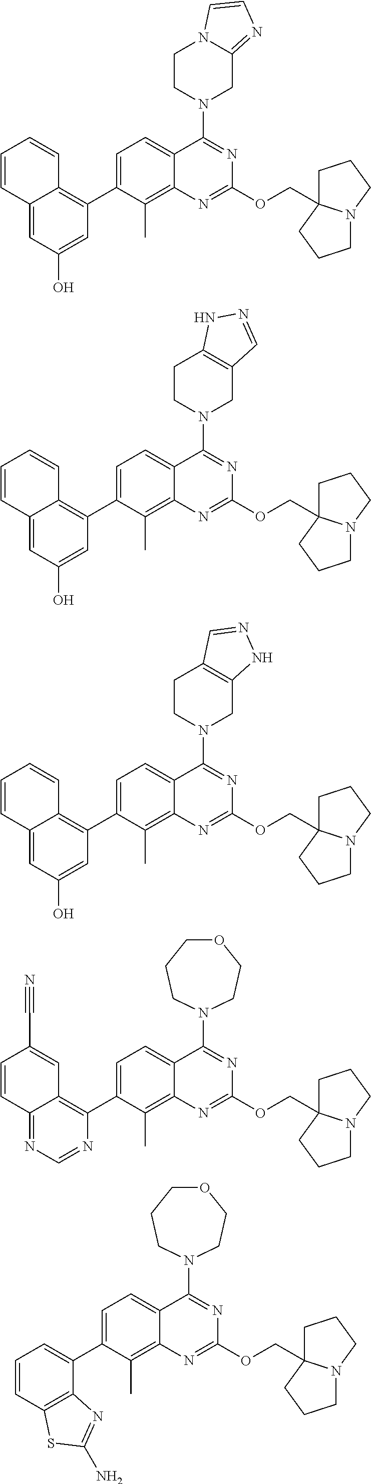 [SMILES: Cc1c(-c2cc(O)cc3ccccc23)ccc2c(N3CCn4ccnc4C3)nc(OCC34CCCN3CCC4)nc12 ; Cc1c(-c2cc(O)cc3ccccc23)ccc2c(N3CCc4[nH]ncc4C3)nc(OCC34CCCN3CCC4)nc12 ; Cc1c(-c2cc(O)cc3ccccc23)ccc2c(N3CCc4cn[nH]c4C3)nc(OCC34CCCN3CCC4)nc12 ; Cc1c(-c2ncnc3ccc(C#N)cc23)ccc2c(N3CCCOCC3)nc(OCC34CCCN3CCC4)nc12 ; Cc1c(-c2cccc3sc(N)nc23)ccc2c(N3CCCOCC3)nc(OCC34CCCN3CCC4)nc12]

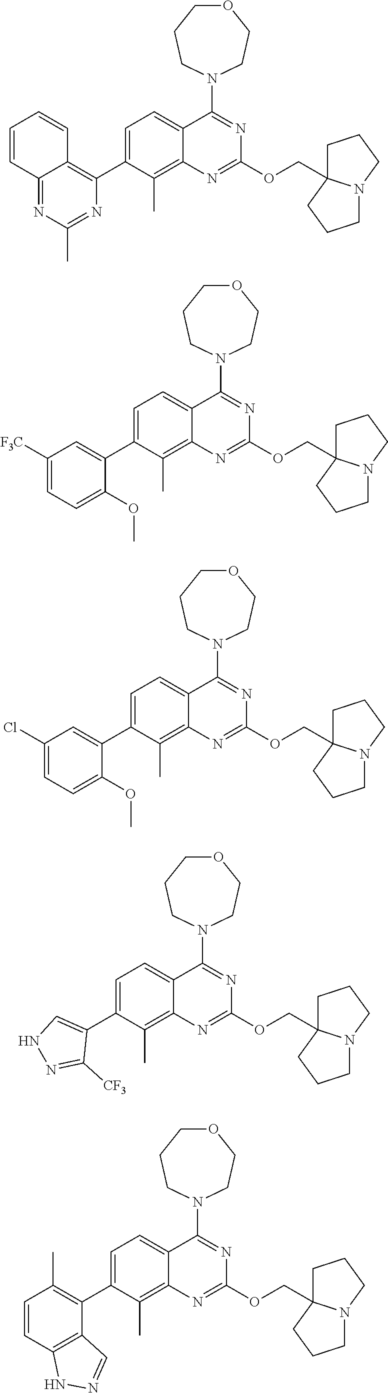 [SMILES: Cc1nc(-c2ccc3c(N4CCCOCC4)nc(OCC45CCCN4CCC5)nc3c2C)c2ccccc2n1 ; COc1ccc(C(F)(F)F)cc1-c1ccc2c(N3CCCOCC3)nc(OCC34CCCN3CCC4)nc2c1C ; COc1ccc(Cl)cc1-c1ccc2c(N3CCCOCC3)nc(OCC34CCCN3CCC4)nc2c1C ; Cc1c(-c2c[nH]nc2C(F)(F)F)ccc2c(N3CCCOCC3)nc(OCC34CCCN3CCC4)nc12 ; Cc1ccc2[nH]ncc2c1-c1ccc2c(N3CCCOCC3)nc(OCC34CCCN3CCC4)nc2c1C]

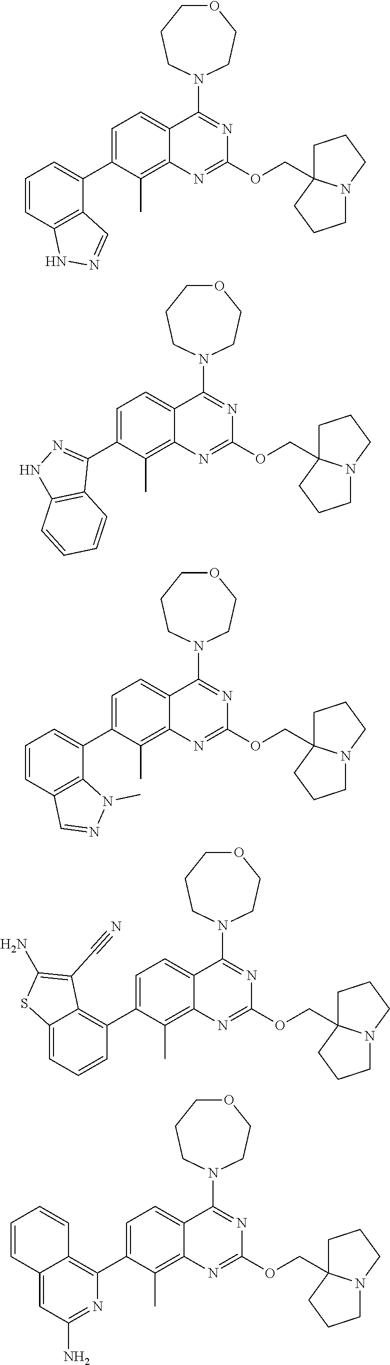 [SMILES: Cc1c(-c2cccc3[nH]ncc23)ccc2c(N3CCCOCC3)nc(OCC34CCCN3CCC4)nc12 ; Cc1c(-c2n[nH]c3ccccc23)ccc2c(N3CCCOCC3)nc(OCC34CCCN3CCC4)nc12 ; Cc1c(-c2cccc3cnn(C)c23)ccc2c(N3CCCOCC3)nc(OCC34CCCN3CCC4)nc12 ; Cc1c(-c2cccc3sc(N)c(C#N)c23)ccc2c(N3CCCOCC3)nc(OCC34CCCN3CCC4)nc12 ; Cc1c(-c2nc(N)cc3ccccc23)ccc2c(N3CCCOCC3)nc(OCC34CCCN3CCC4)nc12]

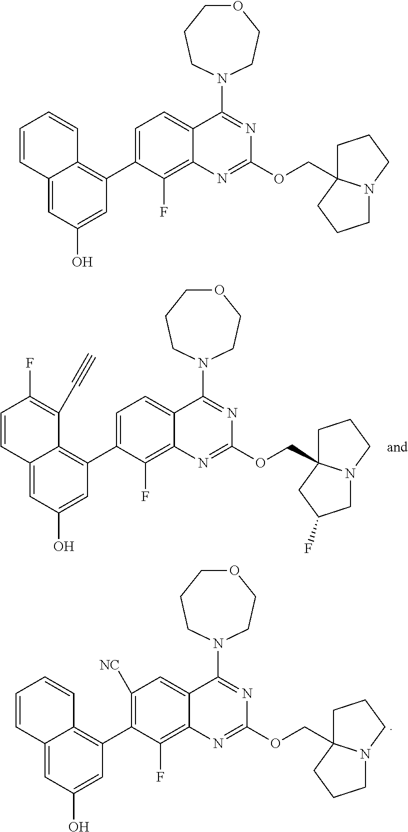 [SMILES: Oc1cc(-c2ccc3c(N4CCCOCC4)nc(OCC45CCCN4CCC5)nc3c2F)c2ccccc2c1 ; C#Cc1c(F)ccc2cc(O)cc(-c3ccc4c(N5CCCOCC5)nc(OC[C@@]56CCCN5C[C@H](F)C6)nc4c3F)c12 ; N#Cc1cc2c(N3CCCOCC3)nc(OCC34CCCN3CCC4)nc2c(F)c1-c1cc(O)cc2ccccc12]

The compound of formula (I) may be:

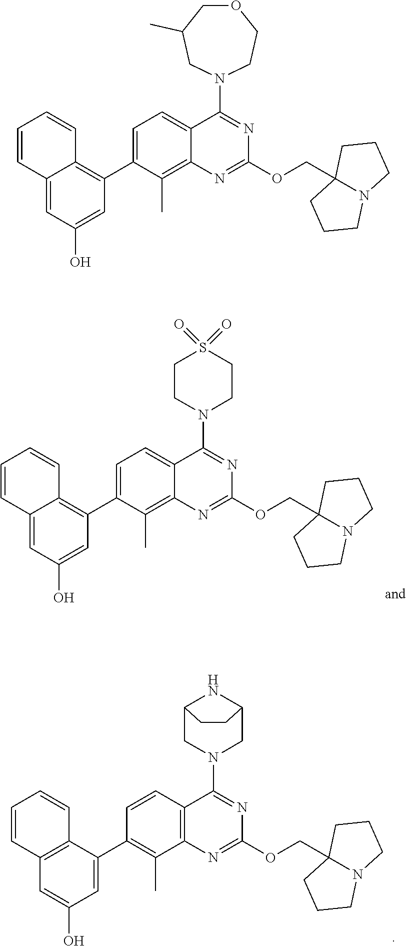 [SMILES: Cc1c(-c2cc(O)cc3ccccc23)ccc2c(N3CCOCC(C)C3)nc(OCC34CCCN3CCC4)nc12 ; Cc1c(-c2cc(O)cc3ccccc23)ccc2c(N3CCS(=O)(=O)CC3)nc(OCC34CCCN3CCC4)nc12 ; Cc1c(-c2cc(O)cc3ccccc23)ccc2c(N3CC4CCC(C3)N4)nc(OCC34CCCN3CCC4)nc12]

The compound of formula (I) may be:

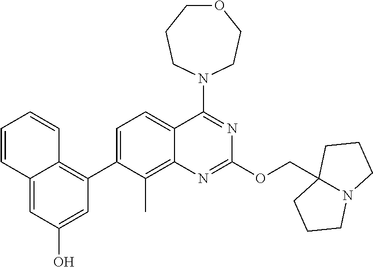 [SMILES: Cc1c(-c2cc(O)cc3ccccc23)ccc2c(N3CCCOCC3)nc(OCC34CCCN3CCC4)nc12]

The compound of formula (I) may be:

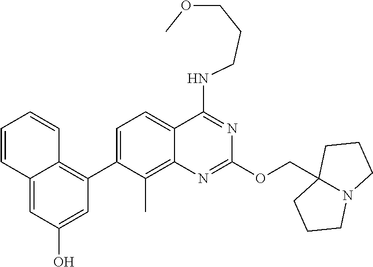 [SMILES: COCCCNc1nc(OCC23CCCN2CCC3)nc2c(C)c(-c3cc(O)cc4ccccc34)ccc12]

The invention also encompasses the subject matter of the following numbered clauses:

1. A compound of formula (I), or a pharmaceutically acceptable salt thereof:

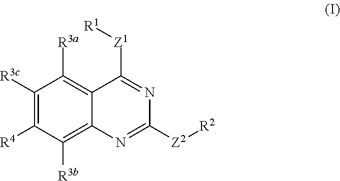

wherein
Z¹ is independently selected from —O— and —NR⁵—;
Z² is independently absent or is selected from —O— and —NR⁶—;
R¹ is independently selected from C₀-C₃-alkylene-R^{1a} and C₂-C₆-alkylene-R^{1b}; wherein R^{1a} is independently selected from a 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups; R^{1b} is independently selected from: NR⁷R⁸ OR⁸, SR⁸, SOR⁸, SO₂R⁸ and SO(NH)R⁸;
or R¹ and R⁵ together with the nitrogen to which they are attached form a ring system selected from: a monocyclic 4- to 7-membered heterocycloalkyl group, optionally substituted with from 1 to 4 R⁹ groups; a fused, spirofused or bridged bicyclic 6- to 11-membered heterocycloalkyl group, optionally substituted with from 1 to 4 R⁹ groups;
R² is independently C₁-C₆-alkyl, C₁-C₄-haloalkyl, C₀-C₄-alkylene-R^{2a}, C₁-C₄-alkylene-R^{2b}, C₂-C₄-alkylene-R²¹;
R^{2a} is independently selected from monocyclic 4- to 7-membered heterocycloalkyl group, a fused, spirofused or bridged bicyclic 6- to 11-membered heterocycloalkyl group; a 5-, 6-, 9- or 10-membered monocyclic or bicyclic heteroaryl group; phenyl; C₃-C₇-cycloalkyl; wherein any heterocycloalkyl or cycloalkyl R^{2a} group is optionally substituted with from 1 to 6 R¹⁰ groups and any heteroaryl or phenyl R^{2a} group is optionally substituted with from 1 to 6 R¹¹ groups;
R^{2b} is independently selected from CONR¹²R¹² and CO₂R¹²;
R^{2b} is independently selected from NR¹²R¹³ and OR¹²
or R² and R⁶ together with the nitrogen to which they are attached form a ring system selected from: monocyclic 4- to 7-membered heterocycloalkyl group, a fused, spirofused or bridged bicyclic 6- to 11-membered heterocycloalkyl group, said heterocycloalkyl group being optionally substituted with from 1 to 6 R¹⁰ groups;
R^{3a}, R^{3b} and R^{3c} are each independently selected from: H, halo, C₁-C₄-alkyl, O—C₁-C₄-alkyl, C₁-C₄-haloalkyl, O—C₁-C₄-haloalkyl, cyclopropyl, nitro and cyano;
R⁴ is independently selected from: phenyl, said phenyl being optionally fused to a C₅-C₇-cycloalkyl ring; naphthyl; and 5-, 6-, 9- or 10-membered monocyclic or bicyclic heteroaryl, wherein R⁴ is optionally substituted with from 1 to 4 R¹⁴ groups;
R⁵, R⁶, R⁸ and R¹² are each independently selected at each occurrence from H, C₁-C₄-haloalkyl, and C₁-C₄-alkyl;
R⁷ and R¹³ are each independently at each occurrence selected from H, C₁-C₄-alkyl, C₁-C₄-haloalkyl and C(O)—C₁-C₄-alkyl;
R⁹ and R¹⁰ are each independently at each occurrence selected from oxo, halo, cyano, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₁-C₄-alkyl substituted with cyano, C₂-C₄-alkenyl, C₂-C₄-alkynyl, C₁-C₄-haloalkyl and cyclopropyl;
R¹¹ and R¹⁴ are each independently at each occurrence selected from halo, cyano, nitro, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₂-C₄-alkenyl, C₂-C₄-alkynyl, C₁-C₄-haloalkyl and cyclopropyl;
wherein any of the aforementioned alkyl, alkylene or cyclopropyl groups is optionally substituted, where chemically possible, by 1 to 5 substituents which are each independently at each occurrence selected from the group consisting of: C₁-C₄-alkyl, halo, nitro, cyano, NR^{a}R^{b}, OR^{a}, SR^{a}, CO₂R^{a}, C(O)R^{a}, CONR^{a}R^{a}; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.
   2. A compound of clause 1, wherein Z¹ is —NR⁵—.
   3. A compound of clause 1 or clause 2, wherein Z² is —O—.
   4. A compound of any one of clauses 1 to 3, wherein R¹ is C₀-C₃-alkylene-R^{1a} wherein R^{1a} is independently selected from a 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups.
   5. A compound of any one of clauses 1 to 3, wherein R¹ and R⁵ together with the nitrogen to which they are attached form a ring system selected from: a monocyclic 4- to 7-membered group heterocycloalkyl group, optionally substituted with from 1 to 4 R⁹ groups; a fused or spirofused bicyclic 6- to 11-membered heterocycloalkyl group, optionally substituted with from 1 to 4 R⁹ groups.
   6. A compound of clause 5, wherein R¹ and R⁵ together with the nitrogen to which they are attached form a ring system having the structure:

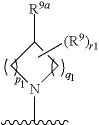

wherein R^{9a} is selected from NR¹²R¹³ and C₁-C₄-alkyl substituted with NR¹²R¹³; p1 is selected from 0, 1, 2 and 3, q1 is selected from 0, 1 and 2; and r1 is selected from 0, 1, 2 and 3.
7. A compound of clause 5, wherein R¹ and R⁵ together with the nitrogen to which they are attached form a ring system having the structure:

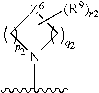

wherein Z⁶ is independently selected from NR^{9b}, O, S, S(O)₂, S(O) and S(O)(NH); R^{9b} is selected from H and C₁-C₄-alkyl; p2 is selected from 2 and 3, q2 is 2; and r2 is selected from 0, 1, 2 and 3.
8. A compound of clause 5, wherein R¹ and R⁵ together with the nitrogen to which they are attached form a ring system having the structure:

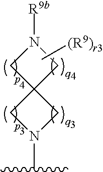

wherein R^{9b} is selected from H and C₁-C₄-alkyl; p3, p4, q3 and q4 are each independently selected from 0, 1, 2 and 3; providing that the sum of p3, p4, q3 and q4 is from 3 to 8, the sum of p3 and q3 is 2 or greater, and the sum of p4 and q4 is 2 or greater; and r3 is selected from 0, 1, 2 and 3.
9. A compound of clause 5, wherein R¹ and R⁵ together with the nitrogen to which they are attached form a ring system having the structure:

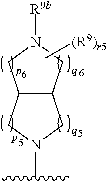

wherein R^{9b} is selected from H and C₁-C₄-alkyl; p5, p6, q5 and are each selected from 0, 1, 2 and 3; providing that the sum of p3, p4, q3 and q4 is from 2 to 7, the sum of p5 and q5 is 1 or greater, and the sum of p6 and q6 is 1 or greater; and r5 is selected from 0, 1, 2 and 3.
10. A compound of clause 5, wherein R¹ and R⁵ together with the nitrogen to which they are attached form a bridged bicyclic 6- to 11-membered heterocycloalkyl group, optionally substituted with from 1 to 4 R⁹ groups.
11. A compound of any one of clauses 1 to 10, wherein R² has the structure:

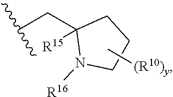

wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; and y is independently selected from 0, 1, 2, 3, and 4.
12. A compound of clause 11, wherein R² has the structure:

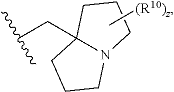

wherein z is independently selected from 0, 1, 2, 3, and 4. z may be selected from 0 and 1. z may be 0. z may be 1.
13. A compound of any one of clauses 1 to 12, wherein R^{3b} is F.
14. A compound of any one of clauses 1 to 13, wherein both R^{3a} and R^{3c} are H.
15. A compound of any one of clauses 1 to 14, wherein R⁴ is phenyl, said phenyl being optionally fused to a C₅-C₇-cycloalkyl ring, wherein R⁴ is optionally substituted with from 1 to 4 R⁴ groups.
16. A compound of any one of clauses 1 to 14, wherein R⁴ has the structure:

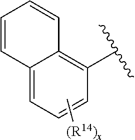

wherein x is independently selected from 0, 1, 2, 3, and 4.
17. A compound of clause 16, wherein R⁴ has the structure:

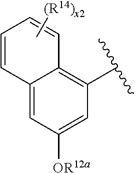

wherein R^{12a} is independently H or C₁-C₄-alkyl; x2 is independently selected from 0, 1, 2 and 3.
18. A compound of any one of clauses 1 to 14, wherein R⁴ is 5-, 6-, 9- or 10-membered monocyclic or bicyclic heteroaryl, optionally substituted with from 1 to 4 R¹⁴ groups.
19. A compound of any one of clause 1 to 18 for medical use.
20. A compound of any one of clause 1 to 18 for use in treating cancer.
21. A compound for use of clause 20, wherein the cancer is selected from: pancreatic carcinoma, colorectal carcinoma, rectal carcinoma, endometrial carcinoma, non-small cell lung carcinoma, gastric carcinoma, ovarian carcinoma and small cell lung carcinoma.
22. A compound for use of clause 20 or 21, wherein the subject being treated has a cancer having the KRAS G12D mutation.
23. A pharmaceutical composition comprising a compound of any one of clauses 1 to 18 and a pharmaceutically acceptable excipient.

### DETAILED DESCRIPTION

In an aspect of the invention there is provided the compounds of the present invention for use as a medicament.

In accordance with another aspect, the present invention provides a method of treating a condition which can be modulated by inhibition of KRAS proteins having the G12D mutation, the method comprising administering a therapeutically effective amount of a compound of the invention to a subject in need thereof.

In accordance with another aspect, the present invention provides a pharmaceutical formulation comprising a compound of the present invention and a pharmaceutically acceptable excipient.

In an embodiment, the pharmaceutical composition may be a combination product comprising an additional pharmaceutically active agent. The additional pharmaceutically active agent may be, for example anti-inflammatory agents, anti-fibrotic agents, chemotherapeutics, anti-cancer agents, immunosuppressants, anti-tumour vaccines, cytokine therapy, or tyrosine kinase inhibitors.

In an aspect of the invention there is provided the compounds of the present invention for use in treating cancer.

In an aspect of the invention there is provided a method of treating cancer, the method comprising administering to a subject in need thereof a therapeutically effective amount of a compound of the invention.

In an aspect of the invention there is provided the use of a compound of the invention for manufacture of a medicament for the treatment of cancer.

The cancer may be a solid tumour or a liquid tumour. The cancer may be a carcinoma.

The cancer may be selected from cervical cancer, endometrial cancer, multiple myeloma, stomach cancer, bladder cancer, uterine cancer, esophageal squamous cell carcinoma, gastric cancer, glioblastomas, astrocytomas; retinoblastoma, osteosarcoma, chondosarcoma, Ewing's sarcoma, rabdomysarcoma, Wilm's tumor, basal cell carcinoma, non-small cell lung cancer, brain tumour, hormone refractory prostate cancer, prostate cancer, metastatic breast cancer, breast cancer, metastatic pancreatic cancer, pancreatic cancer, colorectal cancer, head and neck squamous cell carcinoma, cancer of the head and neck, appendix cancer, cholangiocarcinoma, cancer of unknown primary, ampulla of Vater cancer, ovarian cancer, acute myeloid leukaemia, small cell lung carcinoma, germ cell tumour, small bowel cancer, melanoma, soft tissue sarcoma, gastrointestinal stromal tumour, thyroid cancer, gastrointestinal neuroendocrine tumour, renal cell carcinoma and histiocytosis.

The cancer may be selected from pancreatic carcinoma, colorectal carcinoma, rectal carcinoma, endometrial carcinoma, non-small cell lung carcinoma, gastric carcinoma, ovarian carcinoma and small cell lung carcinoma.

The cancer may have wild-type KRAS. The cancer may have a KRAS mutation. The cancer may have a KRAS mutation selected from: KRAS G12D, KRAS G12C, KRAS G12V, KRAS G12A, KRAS G12S, KRAS G13D and KRAS Q61H. The cancer may have a KRAS G12D mutation. The cancer may have a KRAS G12D mutation, said cancer being selected from pancreatic carcinoma, colorectal carcinoma, rectal carcinoma, endometrial carcinoma, non-small cell lung carcinoma, gastric carcinoma, ovarian carcinoma and small cell lung carcinoma.

The cancer may have a confirmed KRAS G12D mutation. The cancer may have a confirmed KRAS G12D mutation, said cancer being selected from pancreatic carcinoma, colorectal carcinoma, rectal carcinoma, endometrial carcinoma, non-small cell lung carcinoma, gastric carcinoma, ovarian carcinoma and small cell lung carcinoma.

The subject may be human.

The subject may have a cancer with a KRAS G12D mutation. The subject may have a cancer with a KRAS G12D mutation, said cancer being selected from pancreatic carcinoma, colorectal carcinoma, rectal carcinoma, endometrial carcinoma, non-small cell lung carcinoma, gastric carcinoma, ovarian carcinoma and small cell lung carcinoma.

The subject may have a cancer with a confirmed KRAS G12D mutation. The subject may have a cancer with a confirmed KRAS G12D mutation, said cancer being selected from pancreatic carcinoma, colorectal carcinoma, rectal carcinoma, endometrial carcinoma, non-small cell lung carcinoma, gastric carcinoma, ovarian carcinoma and small cell lung carcinoma.

The subject may have a confirmed G12D mutation in their tumour. To be confirmed, the test for G12D presence in the tumour must have >95% for analytical specificity for the detection of mutations in the KRAS gene. Such validated tests would include already commercially available tests i.e. Foundation One CDx and CARIS DNA sequencing.

As mentioned above, the invention includes a method of treating cancer. The method may comprise:
a) confirming that the subject has a cancer with a G12D mutation; and
b) administering to a subject in need thereof a therapeutically effective amount of a compound of the invention.

The term “halo” refers to one of the halogens, group 17 of the periodic table. In particular the term refers to fluorine, chlorine, bromine and iodine. Preferably, the term refers to fluorine or chlorine.

The term “alkyl” refers to a linear or branched hydrocarbon chain. For example, the term “C₁₋₆ alkyl” or “C₁₋₄-alkyl” refers to a linear or branched hydrocarbon chain containing 1, 2, 3, 4, 5, or 6 carbon atoms, for example methyl, ethyl, n-propyl, iso-propyl, n-butyl, sec-butyl, tert-butyl, n-pentyl and n-hexyl. Where an alkyl group is indicated as being C₀₋₄alkyl, then it should be appreciated that this represents the possibility for the alkyl unit to be absent or 1, 2, 3, or 4 carbon atoms in length. Alkylene groups may likewise be linear or branched and may have two places of attachment to the remainder of the molecule. Furthermore, an alkylene group may, for example, correspond to one of those alkyl groups listed in this paragraph. The alkyl and alkylene groups may be unsubstituted or substituted by one or more substituents. Possible substituents are described below. Substituents for the alkyl group may be halogen, e.g. fluorine, chlorine, bromine and iodine, OH, C₁₋₆ alkoxy.

The term “alkoxy” refers to an alkyl group which is attached to a molecule via oxygen. For example, the term “C₁₋₆ alkoxy” refers to an alkyl group which is attached to a molecule via oxygen. This includes moieties where the alkyl part may be linear or branched and may contain 1, 2, 3, 4, 5, or 6 carbon atoms, for example methyl, ethyl, n-propyl, iso-propyl, n-butyl, sec-butyl, tert-butyl, n-pentyl and n-hexyl. Therefore, the alkoxy group may be methoxy, ethoxy, n-propoxy, iso-propoxy, n-butoxy, sec-butoxy, tert-butoxy, n-pentoxy and n-hexoxy. The alkyl part of the alkoxy group may be unsubstituted or substituted by one or more substituents. Possible substituents are described below. Substituents for the alkyl group may be halogen, e.g. fluorine, chlorine, bromine and iodine, OH, C₁₋₆ alkoxy.

The term “haloalkyl” refers to a hydrocarbon chain substituted with at least one halogen atom independently chosen at each occurrence, for example fluorine, chlorine, bromine and iodine. For example, the term “C₁₋₆ haloalkyl” refers to a linear or branched hydrocarbon chain containing 1, 2, 3, 4, 5 or 6 carbon atoms substituted with at least one halogen. The halogen atom may be present at any position on the hydrocarbon chain. For example, C₁₋₆ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl.

The term “alkenyl” refers to a branched or linear hydrocarbon chain containing at least one double bond. For example, the term “C₂₋₆ alkenyl” refers to a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl.

The term “alkynyl” refers to a branched or linear hydrocarbon chain containing at least one triple bond. For example, the term “C₂₋₆ alkynyl” refers to a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl.

The term “heteroalkyl” refers to a branched or linear hydrocarbon chain containing at least one heteroatom selected from N, O and S positioned between any carbon in the chain or at an end of the chain. For example, the term “C₁₋₆ heteroalkyl” refers to a branched or linear hydrocarbon chain containing 1, 2, 3, 4, 5, or 6 carbon atoms and at least one heteroatom selected from N, O and S positioned between any carbon in the chain or at an end of the chain. For example, the hydrocarbon chain may contain one or two heteroatoms. The C₁₋₆ heteroalkyl may be bonded to the rest of the molecule through a carbon or a heteroatom. For example, the “C₁₋₆ heteroalkyl” may be C₁₋₆ N-alkyl, C₁₋₆ N,N-alkyl, or C₁₋₆ O-alkyl.

The term “cycloalkyl” refers to a saturated hydrocarbon ring system. For example, the “C₃₋₈ cycloalkyl” may be cyclopropyl, cyclobutyl, cyclopentyl, cyclohexyl, cycloheptyl and cyclooctyl.

The term “cycloalkenyl” refers to an unsaturated hydrocarbon ring system containing that is not aromatic. The ring may contain more than one double bond provided that the ring 30 system is not aromatic. For example, the “C₃₋₈ cycloalkyl” may be cyclopropenyl, cyclobutenyl, cyclopentenyl, cyclopentadienyl, cyclohexenyl, cyclohexadienyl, cycloheptenyl, cycloheptadiene, cyclooctenyl and cycloatadienyl.

The term “heterocycloalkyl” refers to a saturated hydrocarbon ring system containing carbon atoms and at least one heteroatom within the ring selected from N, O and S. For example, there may be 1, 2 or 3 heteroatoms, optionally 1 or 2. The “heterocycloalkyl” may be bonded to the rest of the molecule through any carbon atom or heteroatom. The “heterocycloalkyl” may have one or more, e.g. one or two, bonds to the rest of the molecule: these bonds may be through any of the atoms in the ring. For example, the “heterocycloalkyl” may be a “C₃₋₈ heterocycloalkyl”. The term “C₃₋₈ heterocycloalkyl” refers to a saturated hydrocarbon ring system containing 3, 4, 5, 6, 7 or 8 carbon atoms and at least one heteroatom within the ring selected from N, O and S. For example, there may be 1, 2 or 3 heteroatoms, optionally 1 or 2. The “C₃₋₈ heterocycloalkyl” may be bonded to the rest of the molecule through any carbon atom or heteroatom. The “C₃₋₈ heterocycloalkyl” may have one or more, e.g. one or two, bonds to the rest of the molecule: these bonds may be through any of the atoms in the ring. For example, the “C₃₋₈ heterocycloalkyl” may be oxirane, aziridine, azetidine, oxetane, tetrahydrofuran, pyrrolidine, imidazolidine, succinimide, pyrazolidine, oxazolidine, isoxazolidine, thiazolidine, isothiazolidine, piperidine, morpholine, thiomorpholine, piperazine, and tetrahydropyran.

The term “heterocycloalkenyl” refers to an unsaturated hydrocarbon ring system that is not aromatic, containing carbon atoms and at least one heteroatom within the ring selected from N, O and S. For example, there may be 1, 2 or 3 heteroatoms, optionally 1 or 2. The “heterocycloalkenyl” may be bonded to the rest of the molecule through any carbon atom or heteroatom. The “heterocycloalkenyl” may have one or more, e.g. one or two, bonds to the rest of the molecule: these bonds may be through any of the atoms in the ring. For example, the “heterocycloalkenyl” may be a “C₃₋₈ heterocycloalkenyl”. The term “C₃₋₈ heterocycloalkenyl” refers to a saturated hydrocarbon ring system containing 3, 4, 5, 6, 7 or 8 atoms at least one of the atoms being a heteroatom within the ring selected from N, O and S. The “heterocycloalkenyl” may be tetrahydropyridine, dihydropyran, dihydrofuran, pyrroline.

The term “fused” refers to a bicyclic ring system in which the two rings are attached via two atoms that are situated adjacent to each other on each ring.

The term “spirofused” refers to a bicyclic ring system in which the two rings are attached via a single atom.

The term “bridged” refers to a bicyclic ring system in which the two rings are attached via two atoms that are not situated adjacent to each other on either ring.

The term “aromatic” when applied to a substituent as a whole means a single ring or polycyclic ring system with 4n+2 electrons in a conjugated π system within the ring or ring system where all atoms contributing to the conjugated π system are in the same plane.

The term “aryl” refers to an aromatic hydrocarbon ring system. The ring system has 4n+2 electrons in a conjugated π system within a ring where all atoms contributing to the conjugated π system are in the same plane. For example, the “aryl” may be phenyl and naphthyl. The aryl system itself may be substituted with other groups.

The term “heteroaryl” refers to an aromatic hydrocarbon ring system with at least one heteroatom within a single ring or within a fused ring system, selected from O, N and S. The ring or ring system has 4n+2 electrons in a conjugated π system where all atoms contributing to the conjugated π system are in the same plane. For example, the “heteroaryl” may be imidazole, thiene, furane, thianthrene, pyrrole, benzimidazole, pyrazole, pyrazine, pyridine, pyrimidine and indole.

The term “halogen” herein includes reference to F, Cl, Br and I. Halogen may be Br. Halogen may be I.

A bond terminating in a “ [symbol] ” represents that the bond is connected to another atom that is not shown in the structure. A bond terminating inside a cyclic structure and not terminating at an atom of the ring structure represents that the bond may be connected to any of the atoms in the ring structure where allowed by valency.

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without inappropriate effort which substitutions are chemically possible, and which are not.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”.

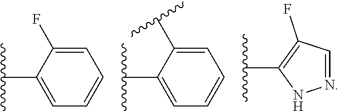

“Meta” substitution is a substitution pattern where two substituents are on carbons one carbon removed from each other, i.e. with a single carbon atom between the substituted carbons. In other words, there is a substituent on the second atom away from the atom with another substituent. For example, the groups below are meta substituted.

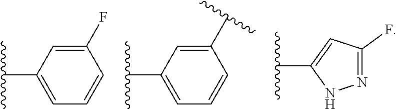

“Para” substitution is a substitution pattern where two substituents are on carbons two carbons removed from each other, i.e with two carbon atoms between the substituted carbons. In other words, there is a substituent on the third atom away from the atom with another substituent. For example, the groups below are para substituted.

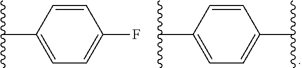

Throughout the description the disclosure of a compound also encompasses pharmaceutically acceptable salts, solvates and stereoisomers thereof. Where a compound has a stereocentre, both (R) and (S) stereoisomers are contemplated by the invention, equally mixtures of stereoisomers or a racemic mixture are completed by the present application. Where a compound of the invention has two or more stereocentres any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%, at least 60% or less. For example, the e.e. or d.e. may be 90% or more, 90% or more, 80% or more, 70% or more, 60% or more, 50% or more, 40% or more, 30% or more, 20% or more, or 10% or more.

The invention contemplates pharmaceutically acceptable salts of the compounds of the invention. These may include the acid addition and base salts of the compounds. These may be acid addition and base salts of the compounds. In addition, the invention contemplates solvates of the compounds. These may be hydrates or other solvated forms of the compound.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:
(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Hereinafter all references to compounds of any formula include references to salts, solvates and complexes thereof and to solvates and complexes of salts thereof.

The compounds of the invention include compounds of a number of formulae as herein defined, including all polymorphs and crystal habits thereof, prodrugs and isomers thereof (including optical, geometric and tautomeric isomers) as hereinafter defined and isotopically-labelled compounds of the invention.

The present invention also includes all pharmaceutically acceptable isotopically-labelled compounds of the invention wherein one or more atoms are replaced by atoms having the same atomic number, but an atomic mass or mass number different from the atomic mass or mass number most commonly found in nature.

Examples of isotopes suitable for inclusion in the compounds of the invention include isotopes of hydrogen, such as ²H and ³H, carbon, such as ¹¹C ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Before purification, the compounds of the present invention may exist as a mixture of enantiomers depending on the synthetic procedure used. The enantiomers can be separated by conventional techniques known in the art. Thus the invention covers individual enantiomers as well as mixtures thereof.

For some of the steps of the process of preparation of the compounds of the invention, it may be necessary to protect potential reactive functions that are not wished to react, and to cleave said protecting groups in consequence. In such a case, any compatible protecting radical can be used. In particular methods of protection and deprotection such as those described by T.W. GREENE (Protective Groups in Organic Synthesis, A. Wiley-Interscience Publication, 1981) or by P. J. Kocienski (Protecting groups, Georg Thieme Verlag, 1994), can be used. All of the above reactions and the preparations of novel starting materials used in the preceding methods are conventional and appropriate reagents and reaction conditions for their performance or preparation as well as procedures for isolating the desired products will be well-known to those skilled in the art with reference to literature precedents and the examples and preparations hereto.

Also, the compounds of the present invention as well as intermediates for the preparation thereof can be purified according to various well-known methods, such as for example crystallization or chromatography.

One or more compounds of the invention may be combined with one or more pharmaceutical agents, for example anti-inflammatory agents, anti-fibrotic agents, chemotherapeutics, anti-cancer agents, immunosuppressants, anti-tumour vaccines, cytokine therapy, or tyrosine kinase inhibitors, for the treatment of conditions modulated by the inhibition of RAS proteins, for example cancer, sarcoma, melanoma, skin cancer, haematological tumors, lymphoma, carcinoma, and leukemia.

The method of treatment or the compound for use in the treatment of cancer, sarcoma, melanoma, skin cancer, haematological tumors, lymphoma, carcinoma, and leukemia as defined hereinbefore may be applied as a sole therapy or be a combination therapy with an additional active agent.

The method of treatment or the compound for use in the treatment of cancer, sarcoma, melanoma, skin cancer, haematological tumors, lymphoma, carcinoma, and leukemiamay involve, in addition to the compound of the invention, additional active agents. The additional active agents may be one or more active agents used to treat the condition being treated by the compound of the invention and additional active agent. The additional active agents may include one or more of the following active agents:—
(i) steroids such as corticosteroids, including glucocorticoids and mineralocorticoids, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids mentioned in this paragraph;
(ii) TNF inhibitors for example etanercept; monoclonal antibodies (e.g. infliximab (Remicade), adalimumab (Humira), certolizumab pegol (Cimzia), golimumab (Simponi)); fusion proteins (e.g. etanercept (Enbrel)); and 5-HT2A agonists (e.g. 2,5-dimethoxy-4-iodoamphetamine, TCB-2, lysergic acid diethylamide (LSD), lysergic acid dimethylazetidide);
(iii) anti-inflammatory drugs, for example non-steroidal anti-inflammatory drugs;
(iv) dihydrofolate reductase inhibitors/antifolates, for example methotrexate, trimethoprim, brodimoprim, tetroxoprim, iclaprim, pemetrexed, ralitrexed and pralatrexate; and
(v) immunosuppressants for example cyclosporins, tacrolimus, sirolimus pimecrolimus, angiotensin II inhibitors (e.g. Valsartan, Telmisartan, Losartan, Irbesatan, Azilsartan, Olmesartan, Candesartan, Eprosartan) and ACE inhibitors e.g. sulfhydryl-containing agents (e.g. Captopril, Zofenopril), dicarboxylate-containing agents (e.g. Enalapril, Ramipril, Quinapril, Perindopril, Lisinopril, Benazepril, Imidapril, Zofenopril, Trandolapril), phosphate-containing agents (e.g. Fosinopril), casokinins, lactokinins and lactotripeptides.
(vi) Anti-fibrotic agents for example: Pirfenidone, Nintedanib, Anti-IL-13 monoclonal antibodies (e.g. Tralokinumab, QAX576, Lebrikizumab), simtuzumab, FG-3019, lysophosphatidic acid receptor antagonists (e.g. BMS-986020, AM966), LOXL2 inhibitors, BET bromodomain inhibitors (e.g. JQ1), HDAC inhibitors (e.g. Vorinostat), thrombin inhibitors (e.g. Dabigatran), FactorXa inhibitors (e.g. Apixban, Rivaroxaban) 15PGDH inhibitors, anti-avP6 monoclonal antibodies (e.g. BG00011), Anti-CTGF monoclonal antibodies (e.g. FG-3019), PAR1 inhibitors, Nox4 inhibitors and PAl-1 inhibitors.
(vii) CNS therapies, for example: Levodopa, Dopamine agonists, Apomorphine, Glutamate antagonist, Anticholinergics, COMT inhibitors, MAO-B inhibitors, riluzole (Rilutek), Tetrabenazine (Xenazine), haloperidol (Haldol), chlorpromazine, risperidone (Risperdal), quetiapine (Seroquel), amantadine, levetiracetam (Keppra), clonazepam (Klonopin), Donepezil (Aricept), Galantamine (Razadyne), Rivastigmine (Exelon)), Memantine (Ebixa, Axura), Aducanumab, Ocrelizumab, interferon beta-1a (Avonex, Rebif), peginterferon beta-1a (Plegridy), teriflunomide (Aubagio), fingolimod (Gilenya), mitoxantrone (Novantrone), dimethyl fumarate (Tecfidera), natalizumab (Tysabri)

The method of treatment or the compound for use in the treatment of cancer, sarcoma, melanoma, skin cancer, haematological tumors, lymphoma, carcinoma, leukemia, and central nervous system disorders may involve, in addition to the compound of the invention, conventional surgery or radiotherapy or chemotherapy. Such chemotherapy may include one or more of the following categories of anti-tumor agents:
(i) antiproliferative/antineoplastic drugs and combinations thereof, such as alkylating agents (for example cis platin, oxaliplatin, carboplatin, cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine); antimetabolites (for example gemcitabine and antifolates such as fluoropyrimidines like 5 fluorouracil and tegafur, raltitrexed, methotrexate, pemetrexed, cytosine arabinoside, floxuridine, cytarabine, 6-mercaptopurine, 6-thioguanine, fludarabine phosphate, pentostatine, and gemcitabine and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, mitoxantrone and camptothecin); bleomcin, dactinomycin, daunorubicin, doxorubicin, epirubicin, idarubicin, ara-C, paclitaxel (Taxol™), nabpaclitaxel, docetaxel, mithramycin, deoxyco-formycin, mitomycin-C, L-asparaginase, interferons (especially IFN-α), etoposide, and teniposide;
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5*-reductase such as finasteride; and navelbene, CPT-II, anastrazole, letrazole, capecitabine, reloxafme, cyclophosphamide, ifosamide, and droloxafine;
(iii) anti-invasion agents, for example dasatinib and bosutinib (SKI-606), and metalloproteinase inhibitors, inhibitors of urokinase plasminogen activator receptor function or antibodies to Heparanase;
(iv) inhibitors of growth factor function: for example such inhibitors include growth factor antibodies and growth factor receptor antibodies, for example the anti erbB2 antibody trastuzumab [Herceptin™], the anti-EGFR antibody panitumumab, the anti erbB1 antibody cetuximab, tyrosine kinase inhibitors, for example inhibitors of the epidermal growth factor family (for example EGFR family tyrosine kinase inhibitors such as gefitinib, erlotinib, 6-acrylamido-N-(3-chloro-4-fluorophenyl)-7-(3-morpholinopropoxy)-quinazolin-4-amine (Cl 1033), erbB2 tyrosine kinase inhibitors such as lapatinib) and antibodies to costimulatory molecules such as CTLA-4, 4-IBB and PD-I, or antibodies to cytokines (IL-10, TGF-beta); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib, tipifarnib and lonafarnib), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor, kinase inhibitors; aurora kinase inhibitors and cyclin dependent kinase inhibitors such as CDK2 and/or CDK4 inhibitors; and CCR2, CCR4 or CCR6 modulator;
(v) antiangiogenic agents such as those which inhibit the effects of vascular endothelial growth factor, for example the anti vascular endothelial cell growth factor antibody bevacizumab (Avastin™); thalidomide; lenalidomide; and for example, a VEGF receptor tyrosine kinase inhibitor such as vandetanib, vatalanib, sunitinib, axitinib and pazopanib;
(vi) gene therapy approaches, including for example approaches to replace aberrant genes such as aberrant p53 or aberrant BRCA1 or BRCA2;
(vii) immunotherapy approaches, including for example antibody therapy such as alemtuzumab, rituximab, ibritumomab tiuxetan (Zevalin®) and ofatumumab; interferons such as interferon α; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); gp100; dendritic cell-based vaccines (such as Ad.p53 DC); and toll-like receptor modulators for example TLR-7 or TLR-9 agonists; and
(viii) cytotoxic agents for example fludaribine (fludara), cladribine, pentostatin (Nipent™);
(ix) steroids such as corticosteroids, including glucocorticoids and mineralocorticoids, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids mentioned in this paragraph;
(x) targeted therapies, for example PI3Kd inhibitors, for example idelalisib and perifosine; PD-1, PD-L1, PD-L2 and CTL4-A modulators, antibodies and vaccines; other IDO inhibitors (such as indoximod); anti-PD-1 monoclonal antibodies (such as MK-3475 and nivolumab); anti-PD-L1 monoclonal antibodies (such as MEDI-4736 and RG-7446); anti-PD-L2 monoclonal antibodies; and anti-CTLA-4 antibodies (such as ipilimumab);
(xii) chimeric antigen receptors, anticancer vaccines and arginase inhibitors.

Such combination treatment may be achieved by way of the simultaneous, sequential or separate dosing of the individual components of the treatment. Such combination products employ the compounds of this invention within a therapeutically effective dosage range described hereinbefore and the other pharmaceutically-active agent within its approved dosage range.

Compounds of the invention may exist in a single crystal form or in a mixture of crystal forms or they may be amorphous. Thus, compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

For the above-mentioned compounds of the invention the dosage administered will, of course, vary with the compound employed, the mode of administration, the treatment desired and the disorder indicated. For example, if the compound of the invention is administered orally, then the daily dosage of the compound of the invention may be in the range from 0.01 micrograms per kilogram body weight (μg/kg) to 100 milligrams per kilogram body weight (mg/kg).

A compound of the invention, or pharmaceutically acceptable salt thereof, may be used on their own but will generally be administered in the form of a pharmaceutical composition in which the compounds of the invention, or pharmaceutically acceptable salt thereof, is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration of the compounds of the invention, the pharmaceutical composition which is used to administer the compounds of the invention will preferably comprise from 0.05 to 99% w (percent by weight) compounds of the invention, more preferably from 0.05 to 80% w compounds of the invention, still more preferably from 0.10 to 70% w compounds of the invention, and even more preferably from 0.10 to 50% w compounds of the invention, all percentages by weight being based on total composition.

The pharmaceutical compositions may be administered topically (e.g. to the skin) in the form, e.g., of creams, gels, lotions, solutions, suspensions, or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); by rectal administration in the form of suppositories; or by inhalation in the form of an aerosol.

For oral administration the compounds of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compounds of the invention may be admixed with, for example, a vegetable oil or polyethylene glycol. Hard gelatine capsules may contain granules of the compound using either the above-mentioned excipients for tablets. Also, liquid or semisolid formulations of the compound of the invention may be filled into hard gelatine capsules. Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For intravenous (parenteral) administration the compounds of the invention may be administered as a sterile aqueous or oily solution.

The size of the dose for therapeutic purposes of compounds of the invention will naturally vary according to the nature and severity of the conditions, the age and sex of the animal or patient and the route of administration, according to well-known principles of medicine.

Dosage levels, dose frequency, and treatment durations of compounds of the invention are expected to differ depending on the formulation and clinical indication, age, and co-morbid medical conditions of the patient.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### EXAMPLES

As used herein the following terms have the meanings given: “Boc” refers to tert-butyloxycarbonyl; “Cbz” refers to carboxybenzyl; “dba” refers to dibenzylideneacetone; “DCM” refers to dichloromethane; “DIPEA” refers to N,N-diisopropylethylamine; “DMA” refers to dimethylacetamide; “DMF” refers to N,N-dimethylformamide; “DMSO” refers to dimethyl sulfoxide; “dppf” refers to 1,1′-bis(diphenylphosphino)ferrocene; “EtOAc” refers to ethyl acetate; “EtOH” refers to ethanol; “Et₂O” refers to diethyl ether; “IPA” refers to isopropyl alcohol; “LiHMDS” refers to lithium bis(trimethylsilyl)amide; “mCPBA” refers to meta-chloroperoxybenzoic acid; “MeCN” refers to acetonitrile; “MeOH” refers to methanol; “min” refers to minutes; “NMR” refers to nuclear magnetic resonance; “PhMe” refers to toluene; “pTsOH” refers to p-toluenesulfonic acid; “py” refers to pyridine; “r.t.” refers to room temperature; “SCX” refers to strong cation exchange; “T3P” refers to propylphosphonic anhydride; “Tf₂O” refers to trifluoromethanesulfonic anhydride; “THF” refers to tetrahydrofuran; “THP” refers to 2-tetrahydropyranyl; “(UP)LC-MS” refers to (ultra performance) liquid chromatography/mass spectrometry.

Solvents, reagents and starting materials were purchased from commercial vendors and used as received unless otherwise described. All reactions were performed at room temperature unless otherwise stated. Compound identity and purity confirmations were performed by LCMS UV using a Waters Acquity SQ Detector 2 (ACQ-SQD2 #LCA081). The diode array detector wavelength was 254 nM and the MS was in positive and negative electrospray mode (m/z: 150-800). A 2 μL aliquot was injected onto a guard column (0.2 μm×2 mm filters) and UPLC column (C18, 50×2.1 mm, <2 μm) in sequence maintained at 40° C. The samples were eluted at a flow rate of 0.6 mL/min with a mobile phase system composed of A (0.1% (v/v) Formic Acid in Water) and B (0.1% (v/v) Formic Acid in Acetonitrile) according to the gradients outlined in Table 1 below. Retention times RT are reported in minutes. The following methods were also used on occasions when described throughout the experimental section, gradients are detailed in table 1. Method 3 utilised a Shimadzu 2020 series spectrometer equipped with a binary pump and diode array detector (acquisition wavelength 214 and 254 nm) and the MS was in positive and negative electrospray mode (m/z: 100-900). 2 μL Aliquot were injected onto an Agilent Poroshell 120 EC-C18 column (2.7 μm, 4.6×50 mm) maintained at 35° C. and eluted at 1.0 ml/min using mobile phase consisting of: A: 0.05% Formic acid in water (v/v), B: 0.05% Formic acid in ACN(v/v). Method 4 utilised a Agilent Technologies 1290 series spectrometer equipped with a binary pump and diode array detector (acquisition wavelength 214 and 254 nm) and the MS was in positive electrospray mode (m/z: 70-1000). 2 μL Aliquots were injected onto an Agilent Eclipse Plus RRHD C18, (1.8 μm, 3.0×50 mm) column maintained at 40° C. and eluted at 0.8 ml/min using mobile phase consisting of: A: 0.05% Formic acid in water (v/v), B: 0.05% Formic acid in ACN(v/v).

TABLE 1

| | | |
|---|---|---|
| Time (min) | % A | % B |

Method 1 (Long acidic)

| | | |
|---|---|---|
| 0 | 95 | 5 |
| 1.1 | 95 | 5 |
| 6.1 | 5 | 95 |
| 7 | 5 | 95 |
| 7.5 | 95 | 5 |
| 8 | 95 | 5 |

Method 2 (Short acidic)

| | | |
|---|---|---|
| 0 | 95 | 5 |
| 0.3 | 95 | 5 |
| 2 | 5 | 95 |
| 2.6 | 95 | 5 |
| 3 | 95 | 5 |

Method 3

| | | |
|---|---|---|
| 0.01 | 85 | 15 |
| 1.0 | 85 | 15 |
| 4.0 | 0 | 100 |
| 4.5 | 0 | 100 |
| 4.51 | 85 | 15 |
| 5.0 | 85 | 15 |

Method 4

| | | |
|---|---|---|
| 0.00 | 80 | 20 |
| 2.65 | 20 | 80 |
| 3.00 | 20 | 80 |

NMR was also used to characterise final compounds. NMR spectra were obtained on a Bruker AVIII 400 Nanobay with 5 mm BBFO probe. Optionally, compound Rf values on silica thin layer chromatography (TLC) plates were measured.

Compound purification was performed by flash column chromatography on silica or by preparative LCMS. LCMS purification was performed using a Waters 3100 Mass detector in positive and negative electrospray mode (m/z: 150-800) with a Waters 2489 UV/Vis detector. Samples were eluted at a flow rate of 20 mL/min on a XBridge™ prep C18 5 μM OBD 19×100 mm column with a mobile phase system composed of A (0.1% (v/v) Formic Acid in Water) and B (0.1% (v/v) Formic Acid in Acetonitrile) according to the gradient outlined in Table 2 below.

TABLE 2

| Time (min) | % A | % B |
|---|---|---|
| 0 | 90 | 10 |
| 1.5 | 90 | 10 |
| 11.7 | 5 | 95 |
| 13.7 | 5 | 95 |
| 14 | 90 | 90 |
| 15 | 90 | 90 |

### General Reaction Schemes

Certain compounds of the invention can be made using the following general reaction schemes. Certain compounds of the invention may be made according to or analogously to the synthetic examples described below.

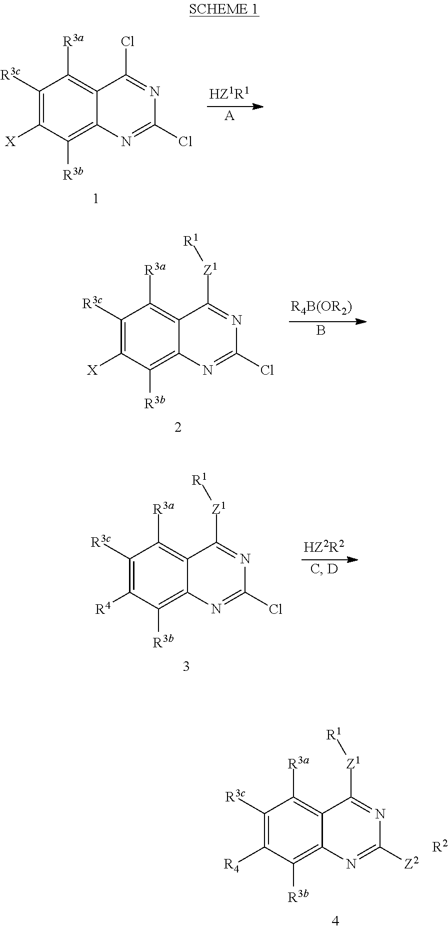

Compounds described by Formula (I) can be prepared according to Scheme 1. In step A compound 1 (where X represents a suitable leaving group such as a halogen or sulfonate ester) can undergo nucleophilic displacement reaction with the appropriate nucleophile HZ¹R¹. R⁴ can then be introduced using a cross-coupling reaction such as a Suzuki reaction in step C with the appropriate boronic acid or ester of R₄. Z²R² can be introduced in step C by nucleophilic displacement of chloride with the appropriate HZ²R² reagent under basic or acidic conditions. Step D may or may not be required and represents a deprotection step to remove protecting groups from reactive atoms present on the R₄, Z₁R₁ and Z₂R₂ groups.

Alternatively, as described in Scheme 2, R⁴ may be introduced prior to the nucleophilic displacement steps (A and B) affording compound 5. From which compound 4 can be prepared in an analogous fashion to that in Scheme 1 through steps A and B followed by deprotection step C as required.

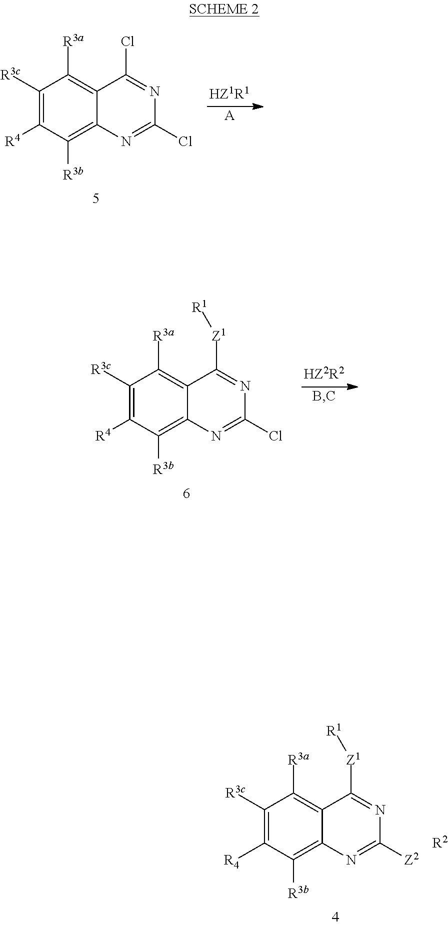

Certain examples were prepared according to scheme 3, starting from commercially available compound 7. Step D was required as a final step to remove the methoxymethyl protecting group introduced in intermediate 12 as well as any acid labile protecting groups present on the Z¹R¹ substituent.

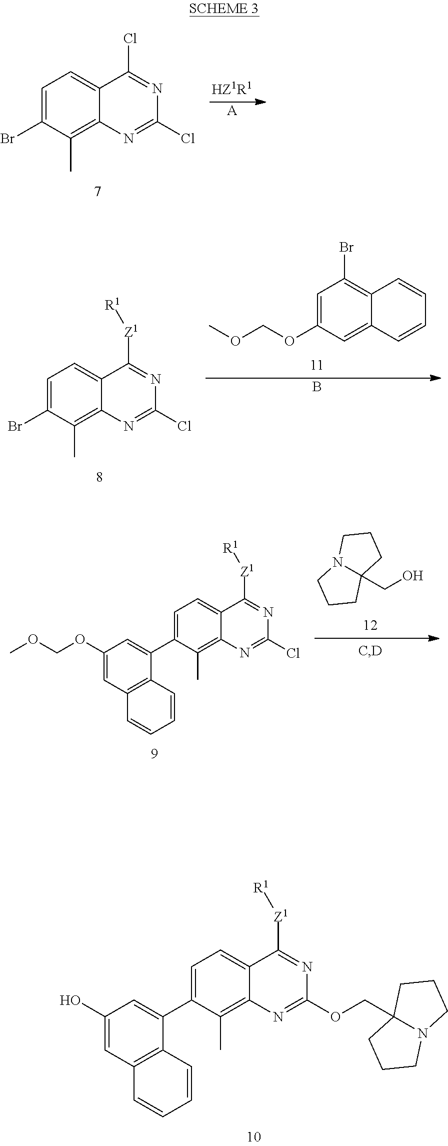

### Intermediate 12—2-[3-(methoxymethoxy)-1-naphthyl]-4,4,5,5-tetramethyl-1,3,2-dioxaborolane

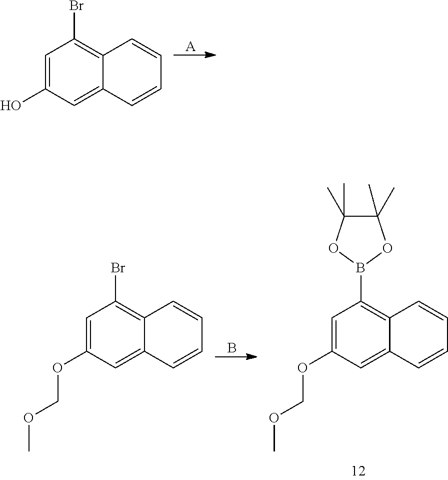

STEP A, 3-(methoxymethoxy)naphthalene: To a suspension of 4-bromonaphthalen-2-ol (3 g, 13.45 mmol) and N,N-diisopropylethylamine (7.03 mL, 40.35 mmol) in DCM (30 mL) at 0° C. was added chloromethyl methyl ether (1.53 mL, 20.17 mmol). The resulting mixture was stirred for 30 min. The reaction was then diluted with distilled water, extracted with DCM (x2), organics were combined and washed with brine (x2), dried over Na₂SO₄, filtered and the filtrate evaporated in vacuo to afford a reddish/pink oil. This was purified by flash column chromatography eluting 0-60% EtOAc in pet. ether (40 g SiO₂, dry loaded with DCM). The desired fractions were combined and evaporated in vacuo to afford a pink oil analysed as 1-bromo-3-(methoxymethoxy)naphthalene (3.2 g, 11.9 mmol, 89% yield).

UPLC-MS (ES⁺, short acidic): 2.06 min, m/z 266.9, 268.9 [M+H]⁺

¹H NMR (400 MHz, CDC₃) δ/ppm: 8.19-8.15 (1H, m), 7.77-7.73 (1H, m), 7.60 (1H, d, J=2.4 Hz), 7.53-7.46 (2H, m), 7.42-7.40 (1H, m), 5.31 (2H, s), 3.55 (3H, s).

STEP B, 2-[3-(methoxymethoxy)-1-naphthyl]-4,4,5,5-tetramethyl-1,3,2-dioxaborolane: To a nitrogen purged suspension of 1-bromo-3-(methoxymethoxy)naphthalene (5.2 g, 19.4 mmol), bis(pinacolato)diboron (9.9 g, 38.9 mmol) and potassium acetate (6.7 g, 68 mmol) in toluene (50 mL) was added [1,1′bis(diphenylphosphino)ferrocene]dichloropalladium (II) (1.4 g, 1.9 mmol). The mixture was stirred at 110° C. for 2.5 hrs. The reaction mixture was then filtered over celite, washing with EtOAc and the filtrate evaporated in vacuo to afford a black oil. This was taken up in distilled water and EtOAc, extracted aqueous (x2) with EtOAc, combined organics were washed with brine, dried over Na₂SO₄, filtered and evaporated in vacuo to afford a black oil. This was purified by flash column chromatography (40 g SiO2, dry loaded with DCM) eluting with 0-20% EtOAc in petrol ether, desired fractions were combined and evaporated in vacuo to afford waxy colourless residue. Analysed as 2-[3-(methoxymethoxy)-1-naphthyl]-4,4,5,5-tetramethyl-1,3,2-dioxaborolane (6 g, 98% yield).

UPLC-MS (ES⁺, short acidic): 2.16 min, m/z 315 [M+H]⁺

¹H NMR (400 MHz, Ds-DMSO) δ/ppm: 8.56-8.59 (1H, d, J=7.1 Hz), 7.86-7.89 (1H, d, J=7.1 Hz), 7.63-7.65 (1H, d, J=2.4 Hz), 7.58-7.60 (1H, d, J=2.4 Hz), 7.40-7.50 (2H, m), 5.31 (2H, s), 3.55 (3H, s), 1.2 (s, 12H).

### Intermediate 11—1,2,3,5,6,7-hexahydropyrrolizin-8-ylmethanol

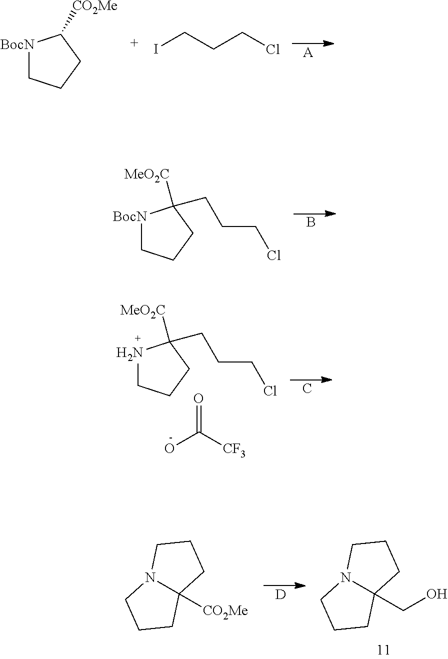

STEP A, O1-tert-butyl O2-methyl 2-(3-chloropropyl)pyrrolidine-1,2-dicarboxylate: A 1M solution of lithium bis(trimethylsilyl)amide (43.6 mL, 43.6 mmol) in THF was added to a solution of N-boc-Proline methylester (10 g, 43.6 mmol) in THF (100 mL) at −78° C. Afterwards, the mixture allowed to stir at that temperature for 30 min. 3-chloropropyl iodide (5.6 mL, 52.3 mmol) was added and reaction mixture was allowed to gradually warm up to 0° C. After 2 hrs in ice bath TLC (3:1 pet. ether:EtOAc) shows complete consumption of starting material (vis. with I₂). Reaction mixture was quenched with a saturated solution of aqueous ammonium chloride (50 mL), partitioned between a layer of ethyl acetate (100 mL) and water (50 mL). The organic layer was separated. The aqueous layer was extracted with ethyl acetate (100 mL), organic extracts were combined, washed with a saturated solution of brine (100 mL), dried over sodium sulfate, filtered and concentrated under reduced pressure to afford a pale yellow oil analysed as O1-tert-butyl O2-methyl 2-(3-chloropropyl)pyrrolidine-1,2-dicarboxylate (13 g, 42.5 mmol, 97% yield).

¹H NMR (400 MHz, CDCl₃) δ/ppm: 3.50-3.62 (4H, m), 3.35-3.45 (2H, m), 3.19-3.23 (1H, m), 2.11-2.20 m, 1H), 1.50-2.05 (m, 6H), 1.25-1.29 (9H, d).

STEP B, methyl 2-(3-chloropropyl)pyrrolidin-1-ium-2-carboxylate; 2,2,2-trifluoroacetate: Trifluoroacetic acid (10 mL, 130.2 mmol) was added to a solution of O1-tert-butyl O2-methyl 2-(3-chloropropyl)pyrrolidine-1,2-dicarboxylate (13 g, 42.5 mmol) in DCM (22 mL). The resulting solution was allowed to stir at room temp overnight. All volatiles removed under reduced pressure and dark oil re-dissolved in DCM and evaporated again to remove TFA. The resulting dark oil was analysed as methyl 2-(3-chloropropyl)pyrrolidin-1-ium-2-carboxylate; 2,2,2-trifluoroacetate (19 g, 59.4 mmol, 140% yield). NMR indicates product is TFA salt and contains approximately 1 equiv. of TFA remains, used without further purification.

¹H NMR (400 MHz, CDCl₃) δ/ppm: 10.20-10.45 (1H, bs), 7.70-8-02 (1H, bs), 3.91 (s, 3H), 3.45-3.70 (m, 4H), 2.45-2.55 (1H, m), 2.26-2.31 (1H, m), 2.15-2.25 (3H, m), 1.95-2.04 (2H, m), 1.55-1.65 (1H, m).

STEP C, methyl 1,2,3,5,6,7-hexahydropyrrolizine-8-carboxylate: Potassium carbonate (27 g, 195.4 mmol) was added to a mixture of potassium iodide (1 g, 6.02 mmol) and methyl 2-(3-chloropropyl)pyrrolidin-1-ium-2-carboxylate; 2,2,2-trifluoroacetate (13.5 g, 42.2 mmol) in methanol (200 mL). The mixture was allowed to stir at 35° C. for 90 mins before concentrating under reduced pressure. Then the reaction mixture was partitioned between a layer of DCM (150 mL) and water (150 mL). The organic layer was separated. The aqueous layer was extracted with DCM (100 mL). The organic extracts were combined, washed with brine (100 ml) dried (Na₂SO₄), filtered and concentrated under reduced pressure to afford methyl 1,2,3,5,6,7-hexahydropyrrolizine-8-carboxylate (6.5 g, 38.4 mmol, 91% yield) as a clear oil.

¹H NMR (400 MHz, CDCl₃) δ/ppm: 3.73 (3H, s), 3.12-3.20 (2H, m), 2.62-2.70 (2H, m), 2.25-2.31 (2H, m), 1.78-1.85 (4H, m), 1.62-1.74 (2H, m).

STEP D, 1,2,3,5,6,7-hexahydropyrrolizin-8-ylmethanol: At 0° C., under an N₂ atmosphere a 1M solution of lithium aluminium hydride (19.5 mL, 19.5 mmol) in THF was added dropwise to a solution of methyl 1,2,3,5,6,7-hexahydropyrrolizine-8-carboxylate (1.1 g, 6.5 mmol) in THF (10 mL). The mixture was allowed to stir at that temperature for 30 mins. Maintaining temp of 0° C., inert atmosphere and using vigorous stirring, the reaction was quenched with dropwise addition of water (0.7 mL) then dropwise addition of a 15% aq. NaOH (0.7 mL) followed by more water (2 ml). The mixture was allowed to stir and warm up to room temperature until the precipitated salts dispersed into a freely moving suspension, before filtering, washing the filter cake with THF (2×10 mL). The filtrate was collected and concentrated under reduced pressure to afford 1,2,3,5,6,7-hexahydropyrrolizin-8-ylmethanol (1.0 g, 7.1 mmol, 100% yield) as a yellow oil.

¹H NMR (400 MHz, CDCl₃) δ/ppm: 3.75-3.79 (1H, m), 3.30 (2H, s), 2.98-3.03 (2H, m), 2.59-2.65 (2H, m), 1.51-1.92 (8H, m).

### Intermediate 13—rac-[(2R,8S)-2-Fluoro-1,2,3,5,6,7-hexahydropyrrolizin-8-yl]methanol

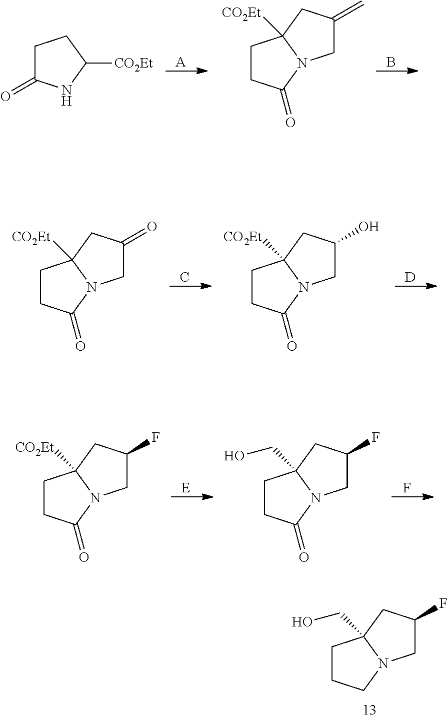

STEP A: Ethyl 6-methylene-3-oxo-1,2,5,7-tetrahydropyrrolizine-8-carboxylate. At −40° C., LiHMDS (1.34 L, 1.34 mol, 2.1 eq) was added dropwise into a solution of ethyl 5-oxo-2-pyrrolidinecarboxylate (100 g, 637 mmol, 1.0 eq) and 3-chloro-2-chloromethyl-1-propene (239 g, 1.91 mol, 3.0 eq) in THF (2000 mL). The mixture was allowed to warm to room temperature and stir over night. The mixture was cooled to −60° C. and adjusted to pH 7 with 2M HCl, poured into water (10 L), extracted with EtOAc (2 L×3), washed with brine (5 L) and dried over Na₂SO₄. The filtered mixture was concentrated and purified by silica gel column (5:1 Pet.ether/EtOAc to 3:1 Pet.ether/EtOAc) to afford ethyl 6-methylene-3-oxo-1,2,5,7-tetrahydropyrrolizine-8-carboxylate as a colourless oil (40 g, 30% yield).

¹H NMR (400 MHz, CDCl₃) δ/ppm: 5.01-5.06 (2H, d), 4.16-4.30 (3H, m), 3.69-3.73 (1H, d), 3.02-3.06 (1H, d), 2.30-2.75 (4H, m), 2.07-2.16 (1H, m), 1.23-1.27 (3H, t).

STEP B: Ethyl 3,6-dioxo-1,2,5,7-tetrahydropyrrolizine-8-carboxylate. At −78° C., O₂ was bubbled through a solution of ethyl 6-methylene-3-oxo-1,2,5,7-tetrahydropyrrolizine-8-carboxylate (115.1 g, 550 mmol, 1.0 eq) in DCM (1 L) and MeOH (100 mL) for 30 minutes. Ozone was then bubbled through the solution with stirring at −78° C. until the solution became blue. O₂ was then bubbled through the solution at the same temperature for a further 30 min. Dimethyl sulfide (68.35 g, 1.1 mol, 2.0 eq) was added at −78° C. and the solution was allowed to reach room temperature and stir over night. The mixture was concentrated and purified by silica gel column (5:1 Pet.ether/EtOAc to 3:1 Pet.ether/EtOAc) to give ethyl 3,6-dioxo-1,2,5,7-tetrahydropyrrolizine-8-carboxylate (108 g, 93% yield) as a colourless oil.

¹H NMR (400 MHz, CDCl₃) δ/ppm: 4.20-4.24 (2H, q), 4.08-4.14 (1H, d), 3.52-3.56 (1H, d), 2.95-3.00 (2H, m), 2.81-2.86 (1H, m), 2.39-2.48 (2H, m), 2.19-2.24 (1H, m), 1.26-1.29 (3H, t).

STEP C: rac-Ethyl (2S,8S)-2-hydroxy-5-oxo-2,3,6,7-tetrahydro-1H-pyrrolizine-8-carboxylate. At 0° C., NaBH₄ (5.81 g, 153 mmol, 0.3 eq) was added to a solution of ethyl 3,6-dioxo-1,2,5,7-tetrahydropyrrolizine-8-carboxylate (108 g, 512 mmol, 1.0 eq) in EtOH (550 mL) and stirred for 10 min. To the mixture was added aqueous NH₄Cl (50 mL) before stirring for a further 20 min at 0° C. The mixture was concentrated in vacuo and the crude was purified by silica gel column (30:1 DCM/MeOH) to afford rac-ethyl (2S,8S)-2-hydroxy-5-oxo-2,3,6,7-tetrahydro-1H-pyrrolizine-8-carboxylate as a yellow oil and apparent mixture of stereoisomers (88 g, 81% yield).

LC-MS (ES⁺, Method 4): 0.40 min, 214.10 [M+H]⁺.

STEP D: rac-Ethyl (2R,8S)-2-fluoro-5-oxo-2,3,6,7-tetrahydro-1H-pyrrolizine-8-carboxylate. At −78° C., DAST (99.8 g, 619 mmol, 1.5 eq) was added dropwise into a solution of rac-ethyl (2S,8S)-2-hydroxy-5-oxo-2,3,6,7-tetrahydro-1H-pyrrolizine-8-carboxylate (88 g, 413 mmol, 1.0 eq) in DCM (1.5 L) and stirred at room temperature overnight. The mixture was cooled to 0° C. before adding MeOH (60 mL) and diluting with brine (2000 mL). The phases were separated and the organics dried over Na₂SO₄, filtered and concentrated under reduced pressure. The crude was purified by silica gel column (8:1 to 5:1 pet. Ether/EtOAc) to give ethyl rac-(2R,8S)-2-fluoro-5-oxo-2,3,6,7-tetrahydro-1H-pyrrolizine-8-carboxylate as a yellow oil (42 g, 47% yield).

¹H NMR (400 MHz, CDCl₃) δ/ppm: 5.21-5.36 (1H, d), 4.20-4.47 (3H, m), 3.19-3.41 (1H, dd), 2.10-2.60 (6H, m), 1.24-1.28 (3H, t).

STEP E: rac-(6R,8S)-6-Fluoro-8-(hydroxymethyl)-2,5,6,7-tetrahydro-1H-pyrrolizin-3-one. At 0° C., LiBH₄ (81 mL, 163 mmol, 1.0 eq) was added dropwise into ethyl (2R,8S)-2-fluoro-5-oxo-2,3,6,7-tetrahydro-1H-pyrrolizine-8-carboxylate (35 g, 163 mmol, 1.0 eq) in THF (350 mL) and stirred at room temperature for 2 hrs. The mixture was cooled to 0° C., before adding aqueous NH₄Cl (100 mL) and stirring for 30 mins at 0° C., the mixture was concentrated and the crude was purified by silica gel column (30:1 DCM/MeOH) to give rac-(6R,8S)-6-fluoro-8-(hydroxymethyl)-2,5,6,7-tetrahydro-1H-pyrrolizin-3-one (28 g, 99% yield).

¹H NMR (400 MHz, CDCl₃) δ/ppm: 5.21-5.35 (1H, d), 4.05-4.16 (1H, m), 3.51-3.55 (1H, d), 3.42-3.46 (1H, d) 2.52-3.11 (3H, m), 1.94-2.41 (5H. m).

STEP F: rac-[(2R,8S)-2-Fluoro-1,2,3,5,6,7-hexahydropyrrolizin-8-yl]methanol. At 0° C., BH₃·DMS (10M, 75.1 mL, 751 mmol, 5.0 eq) was added dropwise into rac-(6R,8S)-6-fluoro-8-(hydroxymethyl)-2,5,6,7-tetrahydro-1H-pyrrolizin-3-one (26 g, 150 mmol, 1.0 eq) in THF (1300 mL) and stirred at room temperature overnight. The mixture was cooled to 0° C., MeOH (300 mL) was added and stirring continued for 1 hr at 0° C. The mixture was concentrated and the crude was dissolved in MeOH (300 mL) and stirred at 50° C. overnight. The mixture was concentrated to give (21 g, 87.9% yield) of rac-[(2R,8S)-2-fluoro-1,2,3,5,6,7-hexahydropyrrolizin-8-yl]methanol, intermediate 21 as a colourless oil.

¹H NMR (400 MHz, CDCl₃) δ/ppm: 5.11-525 (1H, d), 2.80-3.31 (6H, m), 1.74-2.11 (6H, m).

Intermediate 14 4-[7-bromo-2-(1,2,3,5,6,7-hexahydropyrrolizin-8-ylmethoxy)-8-methyl-quinazolin-4-yl]-1,4-oxazepane and intermediate 15 4-[2-(1,2,3,5,6,7-hexahydropyrrolizin-8-ylmethoxy)-8-methyl-7-(4,4,5,5-tetramethyl-1,3,2-dioxaborolan-2-yl)quinazolin-4-yl]-1,4-oxazepane

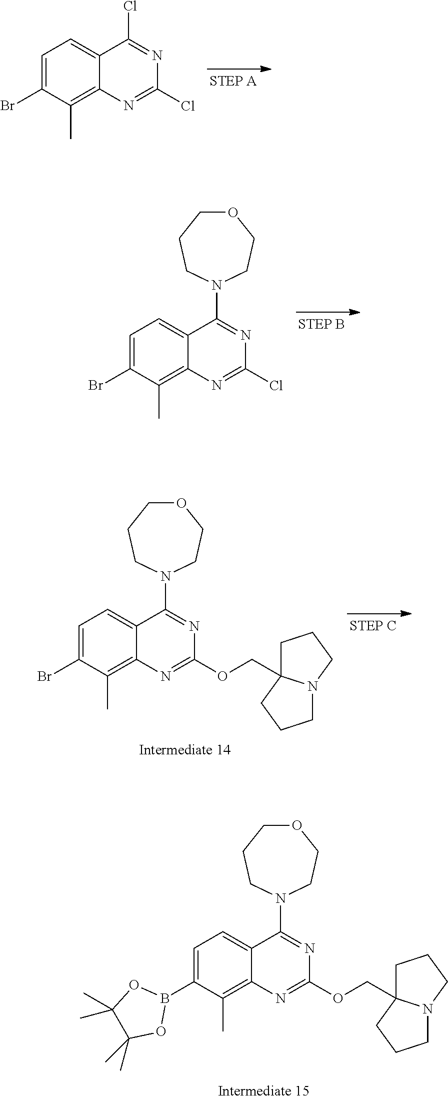 [SMILES: Cc1c(Br)ccc2c(Cl)nc(Cl)nc12 ; Cc1c(Br)ccc2c(N3CCCOCC3)nc(Cl)nc12 ; Cc1c(Br)ccc2c(N3CCCOCC3)nc(OCC34CCCN3CCC4)nc12 ; Cc1c(B2OC(C)(C)C(C)(C)O2)ccc2c(N3CCCOCC3)nc(OCC34CCCN3CCC4)nc12]

STEP A, 4-(7-bromo-2-chloro-8-methyl-quinazolin-4-yl)-1,4-oxazepane: To a solution of 7-bromo-2,4-dichloro-8-methylquinazoline (200 mg, 0.69 mmol) and [1,4]-oxazepane (139 mg, 1.37 mmol) in DCM (7 mL) at 0° C. was added n,n-diisopropylethylamine (0.6 mL, 3.43 mmol) and the mixture was stirred at 0° C. for 2 hours. The reaction mixture was dry loaded onto silica and the crude was purified by column chromatography eluting 0-50% ethyl acetate in petroleum ether. The desired fractions were concentrated in vacuo to yield 4-(7-bromo-2-chloro-8-methyl-quinazolin-4-yl)-1,4-oxazepane (206 mg, 0.58 mmol, 84% yield) as a white solid.

UPLC-MS (ES⁺, Short acidic): 2.22 min. m/z 358.0 [M+H]⁺.

¹H NMR (400 MHz, DMSO-d6) δ/ppm: 7.88 (d, J=8.7 Hz, 1H), 7.64 (d, J=9.1 Hz, 1H), 4.04-4.00 (m, 4H), 3.88-3.86 (m, 2H), 3.72-3.69 (m, 2H), 2.62 (s, 3H), 2.07-2.01 (m, 2H).

STEP B, 4-[7-bromo-2-(1,2,3,5,6,7-hexahydropyrrolizin-8-ylmethoxy)-8-methyl-quinazolin-4-yl]-1,4-oxazepane: Sodium hydride, 60% dispersed in mineral oil (69 mg, 1.73 mmol) was added to 1,2,3,5,6,7-hexahydropyrrolizin-8-ylmethanol (163 mg, 1.16 mmol) in dry THF (6 mL) at 0° C. under nitrogen and the reaction was stirred at 0° C. for 30 minutes. 4-(7-Bromo-2-chloro-8-methyl-quinazolin-4-yl)-1,4-oxazepane (206 mg, 0.58 mmol) was added and the vial was sealed. The reaction was heated to 60° C. and stirred overnight. Water (10 mL) was added and the reaction was extracted with ethyl acetate (10 mL). The organics were washed with NaHCO₃, dried over Na₂SO₄ and concentrated in vacuo to yield 4-[7-bromo-2-(1,2,3,5,6,7-hexahydropyrrolizin-8-ylmethoxy)-8-methyl-quinazolin-4-yl]-1,4-oxazepane (245 mg, 0.53 mmol, 92% yield) as a pink solid.

UPLC-MS (ES⁺, Short acidic): 1.38 min, m/z 463.1 [M+H]⁺

¹H NMR (400 MHz, DMSO-d6) δ/ppm: 7.66 (d, J=9.0 Hz, 1H), 7.42 (d, J=9.1 Hz, 1H), 4.04 (s, 2H), 3.99-3.96 (m, 4H), 3.87-3.85 (m, 2H), 3.71 (t, J=5.3 Hz, 2H), 2.96-2.91 (m, 2H), 2.61 (s, 3H), 2.57-2.54 (m, 1H), 2.07-2.01 (m, 2H), 1.93-1.70 (m, 7H), 1.60-1.53 (m, 2H).

STEP C, 15 4-[2-(1,2,3,5,6,7-hexahydropyrrolizin-8-ylmethoxy)-8-methyl-7-(4,4,5,5-tetramethyl-1,3,2-dioxaborolan-2-yl)quinazolin-4-yl]-1,4-oxazepane. To a degassed suspension of 4-[7-bromo-2-(1,2,3,5,6,7-hexahydropyrrolizin-8-ylmethoxy)-8-methyl-quinazolin-4-yl]-1,4-oxazepane (500 mg, 1.08 mmol), bis(pinacolato)diboron (550.37 mg, 2.17 mmol) and potassium acetate (372 mg, 3.79 mmol) in toluene (12 mL) was added [1,1′-bis(diphenylphosphino)ferrocene]dichloropalladium II (79.29 mg, 0.11 mmol). The mixture was stirred at 110° C. for 2 hours before diluting in EtOAc and filtering. The filtrate was evaporated in vacuo to afford an orange oil. The product was purified by flash column chromatography (KP Amino D 11 g) eluting 0-100% EtOAc in petrol ether. The desired fractions were combined and concentrated in vacuo to afford 4-[2-(1,2,3,5,6,7-hexahydropyrrolizin-8-ylmethoxy)-8-methyl-7-(4,4,5,5-tetramethyl-1,3,2-dioxaborolan-2-yl)quinazolin-4-yl]-1,4-oxazepane as a crude brown oil used without further purification (380 mg, 69%).

UPLC-MS (ES⁺, Short acidic): 1.63 min, m/z 509.3[M+H]⁺

### Intermediate 16

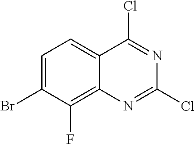 [SMILES: Fc1c(Br)ccc2c(Cl)nc(Cl)nc12]

### 7-bromo-2,4-dichloro-8-fluoro-quinazoline

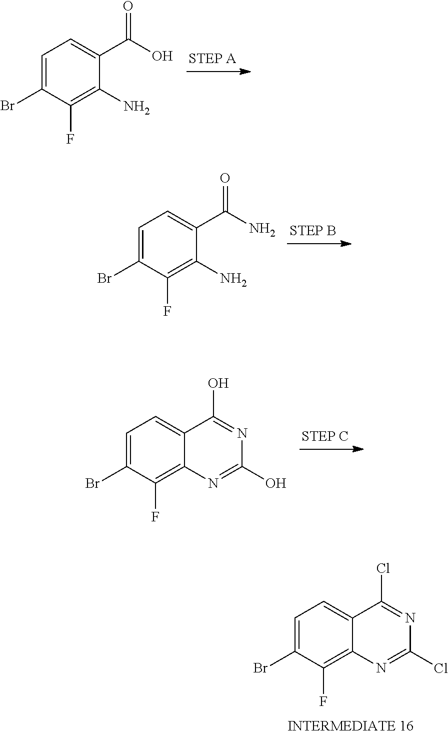 [SMILES: Nc1c(C(=O)O)ccc(Br)c1F ; NC(=O)c1ccc(Br)c(F)c1N ; Oc1nc(O)c2ccc(Br)c(F)c2n1 ; Fc1c(Br)ccc2c(Cl)nc(Cl)nc12]

STEP A, 2-amino-4-bromo-3-fluoro-benzamide. To a solution of 2-amino-4-bromo-3-fluorobenzoic acid (7 g, 29.9 mmol) in DMF (70 mL) was added DIEA (38.6 g, 299.1 mmol) and HATU (22.7 g59.824 mmol), NH₄Cl (9.5 g, 179.472 mmol).The mixture was stirred at room temperature for 2 hrs. The mixture was diluted with MTBE (1000 mL), washed 1 M HCl (500 mL) and brine (500 mL) and dried over Na₂SO₄ and concentrated under vacuum before triturating with DCM to give 2-amino-4-bromo-3-fluoro-benzamide (5 g, 70%) as a light red solid.

UPLC-MS (ES⁺, Method 4): 1.2 min, m/z 233.0 and 235.0 [M+H]⁺

¹H NMR (400 MHz, DMSO-d6) δ/ppm: 7.91 (bs. 1H), 7.37-7.35 (m, 2H), 6.78-6.74 (m, 3H).

STEP B, 7-bromo-8-fluoro-quinazoline-2,4-diol. To a solution of 2-amino-4-bromo-3-fluorobenzamide (9 g, 38.6 mmol) in DMF (150 mL) was added NaH (3.86 g, 96 mmol, 60% in oil) at 0° C., and stirred at 0° C. for 30 mins. CDI (15.6 g, 96.5 mmol) was added and the mixture was stirred at 60° C. for 30 mins. The mixture was quenched with water and adjusted pH to 2 with 1 M HCl, filtered and washed with water to get 7-bromo-8-fluoro-quinazoline-2,4-diol (11.5 g) as light yellow solid.

UPLC-MS (ES⁺, Method 4): 1.0 min, m/z 256.9 and 259.0 [M−H]⁺

¹H NMR (400 MHz, DMSO-d6) δ/ppm: 11.51-11.46 (m, 2H), 7.64-7.61 (m, 1H), 7.44-7.41 (m, 1H).

STEP C, 7-bromo-2,4-dichloro-8-fluoro-quinazoline. A mixture of 7-bromo-8-fluoro-quinazoline-2,4-diol (200 mg, 0.77 mmol), DIEA (499 mg, 3.86 mmol) and POCl₃ (2.1 g, 13.9 mmol) was stirred at 100° C. for 0.5 hrs. The reaction mixture was concentrated in vacuo and diluted with saturated aqueous NaHCO₃ (10 ml). The aqueous layer was extracted with DCM (5 ml) and washed with brine (2×10 ml). The organic layer was dried over Na₂SO₄ and concentrated in vacuo to give crude 7-bromo-2,4-dichloro-8-fluoro-quinazoline (188 mg, 82%) used without further purification.

¹H NMR (400 MHz, DMSO-d6) δ/ppm: 7.65-7.62 (m, 1H), 7.46-7.42 (m, 1H).

### EXAMPLE COMPOUNDS

### Example 1

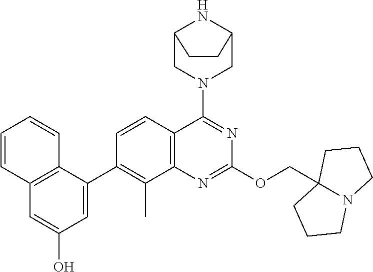 [SMILES: Cc1c(-c2cc(O)cc3ccccc23)ccc2c(N3CC4CCC(C3)N4)nc(OCC34CCCN3CCC4)nc12]

### 4-[4-(3,8-diazabicyclo[3.2.1]octan-3-yl)-2-(1,2,3,5,6,7-hexahydropyrrolizin-8-ylmethoxy)-8-methyl-quinazolin-7-yl]naphthalen-2-ol

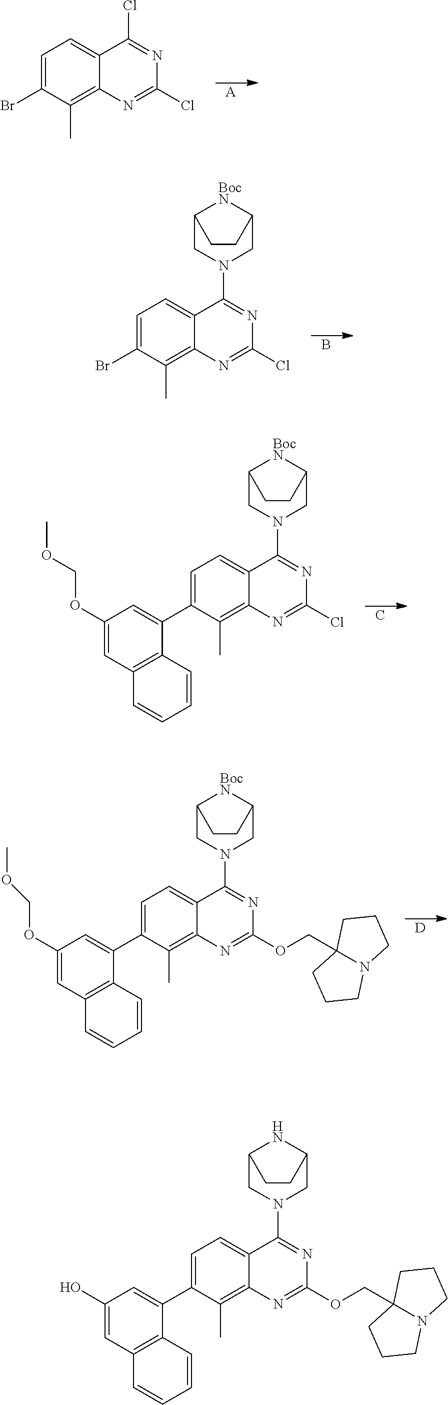

STEP A, tert-butyl 3-(7-bromo-2-chloro-8-methyl-quinazolin-4-yl)-3,8-diazabicyclo[3.2.1]octane-8-carboxylate: A solution of tert-butyl 3-(7-bromo-2-chloro-8-methyl-quinazolin-4-yl)-3,8-diazabicyclo[3.2.1]octane-8-carboxylate (80 mg, 0.17 mmol) in NMP (1.5 mL) was added to 1,2,3,5,6,7-hexahydropyrrolizin-8-ylmethanol (45 mg, 0.32 mmol). The reaction was heated to 140° C. for 2 hrs under microwave irradiation. The mixture was diluted into EtOAc and water and the layers separated. The organic layer was washed with water (x3), NH₄Cl (x2) and brine (x3) and dried (Na₂SO₄). The solution was concentrated in vacuo to afford tert-butyl 3-(7-bromo-2-chloro-8-methyl-quinazolin-4-yl)-3,8-diazabicyclo[3.2.1]octane-8-carboxylate in quantitative yield as a brown oil.

UPLC-MS (ES⁺, Short acidic): 2.36 min, m/z 469.1 [M+H]⁺

¹H NMR (400 MHz, CDCl₃) δ/ppm: 7.54-7.48 (m, 2H), 4.38-4.28 (m, 2H), 3.68-3.47 (m, 2H), 3.11-2.97 (m. 1H), 2.75 (s, 3H), 2.53-1.41 (m, 1H), 1.98-1.88 (m, 2H), 1.80-1.70 (m, 2H), 1.02 (s, 9H) STEP B, tert-butyl 3-[2-chloro-7-[3-(methoxymethoxy)-1-naphthyl]-8-methyl-quinazolin-4-yl]-3,8-diazabicyclo[3.2.1]octane-8-carboxylate: A stirred solution of tert-butyl 3-(7-bromo-2-chloro-8-methyl-quinazolin-4-yl)-3,8-diazabicyclo[3.2.1]octane-8-carboxylate (145.03 mg, 0.3100 mmol), 2-[3-(methoxymethoxy)-1-naphthyl]-4,4,5,5-tetramethyl-1,3,2-dioxaborolane (107 mg, 0.34 mmol) and cesium carbonate (202 mg, 0.62 mmol) in 1,4-dioxane (3 mL) and water (0.5 mL) was degassed with N₂ for 10 minutes.

Tris(dibenzylideneacetone)dipalladium(0) (28 mg, 0.0300 mmol) was added and the reaction was stirred at 100° C. overnight. The reaction was filtered through celite, washing with MeOH. The product was purified by flash column chromatography (12 g SiO₂, 0-40% EtOAc in pet. ether). The desired fractions were concentrated in vacuo to afford tert-butyl 3-[2-chloro-7-[3-(methoxymethoxy)-1-naphthyl]-8-methyl-quinazolin-4-yl]-3,8-diazabicyclo[3.2.1]octane-8-carboxylate (116 mg, 0.20 mmol, 65% yield) as a colourless oil.

UPLC-MS (ES⁺, Short acidic): 2.43 min, m/z 576.3 [M+H]

¹H NMR (400 MHz, CDCl₃) δ/ppm 7.83 (d, J=8.2 Hz, 1H), 7.75 (d, J=9.0 Hz, 1H), 7.49-7.44 (m, 2H), 7.32-7.29 (m, 2H), 7.25-7.24 (m, 1H), 7.10 (d, J=2.6 Hz, 1H), 5.36-5.32 (m, 2H), 4.48-4.43 (m, 2H), 3.56 (s, 3H), 2.38 (s, 3H), 2.01-1.85 (m, 4H), 1.52 (s, 9H), 1.32-1.24 (m, 4H).

STEP C, tert-butyl 3-[2-(1,2,3,5,6,7-hexahydropyrrolizin-8-ylmethoxy)-7-[3-(methoxymethoxy)-1-naphthyl]-8-methyl-quinazolin-4-yl]-3,8-diazabicyclo[3.2.1]octane-8-carboxylate: To a stirred solution of 1,2,3,5,6,7-hexahydropyrrolizin-8-ylmethanol (23 mg, 0.16 mmol) in THF (1 mL) at 0° C. was added sodium hydride, (60% dispersed in mineral oil) (10 mg, 0.26 mmol). The solution was stirred at 0° C. for 30 mins. The ice bath was removed and tert-butyl 3-[2-chloro-7-[3-(methoxymethoxy)-1-naphthyl]-8-methyl-quinazolin-4-yl]-3,8-diazabicyclo[3.2.1]octane-8-carboxylate (50 mg, 0.09 mmol) was added. The vial was sealed and heated to 60° C. for 4 hours. The reaction was cooled to room temperature and concentrated in vacuo prior to partitioning between EtOAc and water. The layers were separated and organic layer was washed with water (x3). The organics were passed through a phase separator and concentrated in vacuo to afford tert-butyl 3-[2-(1,2,3,5,6,7-hexahydropyrrolizin-8-ylmethoxy)-7-[3-(methoxymethoxy)-1-naphthyl]-8-methyl-quinazolin-4-yl]-3,8-diazabicyclo[3.2.1]octane-8-carboxylate (60 mg, 0.09 mmol, 100% yield) as an orange solid.

UPLC-MS (ES⁺, Short acidic): 1.95 min, m/z 680.8 [M+H]⁺ (84%)

STEP D, 4-[4-(3,8-diazabicyclo[3.2.1]octan-3-yl)-2-(1,2,3,5,6,7-hexahydropyrrolizin-8-ylmethoxy)-8-methyl-quinazolin-7-yl]naphthalen-2-ol: At 0° C., triethylsilane (0.11 mL, 0.69 mmol) was added to a solution of tert-butyl 3-[2-(1,2,3,5,6,7-hexahydropyrrolizin-8-ylmethoxy)-7-[3-(methoxymethoxy)-1-naphthyl]-8-methyl-quinazolin-4-yl]-3,8-diazabicyclo[3.2.1]octane-8-carboxylate (47 mg, 0.07 mmol) in DCM (0.7 mL). Trifluoroacetic acid (1 mL, 13.75 mmol) was added and reaction mixture allowed to stir at room temperature for 1 hr. The reaction mixture was concentrated under reduced pressure and dry loaded onto celite. The product was purified by reverse phase chromatography (4 g C-18 silica, 15-100% MeCN in water) and the desired fractions concentrated in vauco. Product further purified by SCX (1 g cartridge), eluting with MeOH (x2 vol.) followed by 1M NH₃ in MeOH (x3 vol.) affording 4-[4-(3,8-diazabicyclo[3.2.1]octan-3-yl)-2-(1,2,3,5,6,7-hexahydropyrrolizin-8-ylmethoxy)-8-methyl-quinazolin-7-yl]naphthalen-2-ol (14 mg, 0.026 mmol, 38% yield) as a white solid.

UPLC-MS (ES⁺, Long acidic): 2.48 min, m/z 536.4 [M+H]⁺

¹H NMR (400 MHz, DMSO-d6) δ/ppm: 9.89 (s, 1H), 7.85 (d, J=8.6 Hz, 1H), 7.78 (d, J=8.5 Hz, 1H), 7.44-7.38 (m, 1H), 7.21 (d, J=2.5 Hz, 1H), 7.19-7.17 (m, 2H), 7.10 (d, J=8.9 Hz, 1H), 6.95 (d, J=2.4 Hz, 1H), 4.24 (t, J=10.0 Hz, 2H), 4.05 (s, 2H), 3.50 (s, 2H), 3.43 (q, J=6.3 Hz, 2H), 2.94 (dq, J=5.3, 5.1 Hz, 2H), 2.17 (s, 3H), 1.96-1.89 (m, 3H), 1.85-1.72 (m, 6H), 1.70-1.64 (m, 2H), 1.62-1.54 (m, 3H).

### Example 2

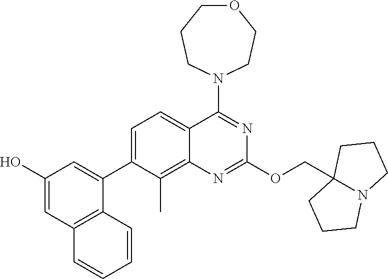 [SMILES: Cc1c(-c2cc(O)cc3ccccc23)ccc2c(N3CCCOCC3)nc(OCC34CCCN3CCC4)nc12]

### 4-[2-(1,2,3,5,6,7-hexahydropyrrolizin-8-ylmethoxy)-8-methyl-4-(1,4-oxazepan-4-yl)quinazolin-7-yl]naphthalen-2-ol

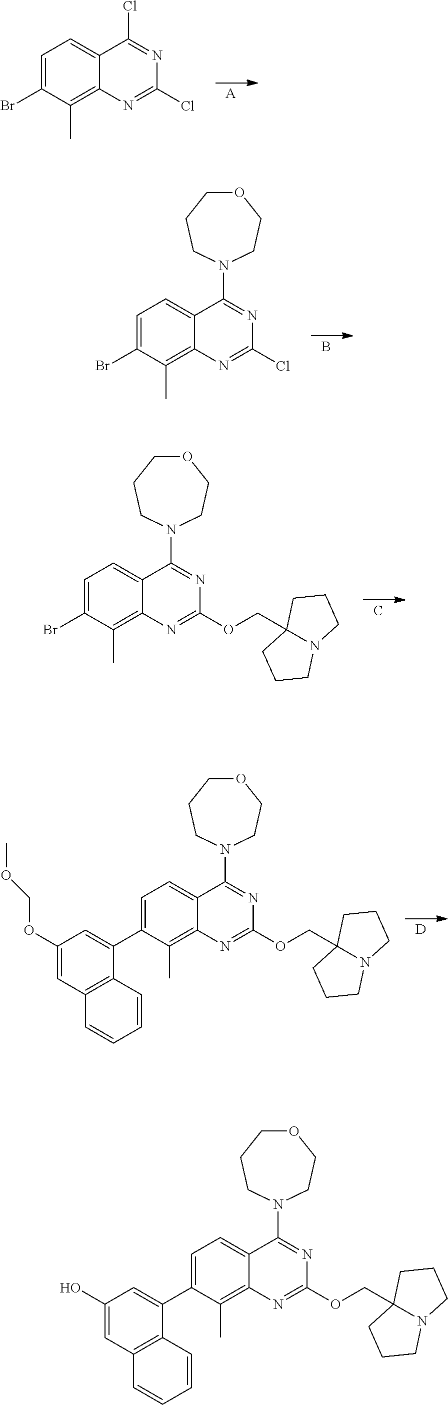

STEP A, 4-(7-bromo-2-chloro-8-methyl-quinazolin-4-yl)-1,4-oxazepane: To a solution of 7-bromo-2,4-dichloro-8-methylquinazoline (200 mg, 0.69 mmol) and [1,4]-oxazepane (139 mg, 1.37 mmol) in DCM (7 mL) at 0° C. was added n,n-diisopropylethylamine (0.6 mL, 3.43 mmol) and the mixture was stirred at 0° C. for 2 hours. The reaction mixture was dry loaded onto silica and the crude was purified by column chromatography eluting 0-50% ethyl acetate in petroleum ether. The desired fractions were concentrated in vacuo to yield 4-(7-bromo-2-chloro-8-methyl-quinazolin-4-yl)-1,4-oxazepane (206 mg, 0.58 mmol, 84% yield) as a white solid.

UPLC-MS (ES⁺, Short acidic): 2.22 min, m/z 358.0 [M+H]⁺.

¹H NMR (400 MHz, DMSO-d6) δ/ppm: 7.88 (d, J=8.7 Hz, 1H), 7.64 (d, J=9.1 Hz, 1H), 4.04-4.00 (m, 4H), 3.88-3.86 (m, 2H), 3.72-3.69 (m, 2H), 2.62 (s, 3H), 2.07-2.01 (m, 2H).

STEP B, 4-[7-bromo-2-(1,2,3,5,6,7-hexahydropyrrolizin-8-ylmethoxy)-8-methyl-quinazolin-4-yl]-1,4-oxazepane: Sodium hydride, 60% dispersed in mineral oil (69 mg, 1.73 mmol) was added to 1,2,3,5,6,7-hexahydropyrrolizin-8-ylmethanol (163 mg, 1.16 mmol) in dry THF (6 mL) at 0° C. under nitrogen and the reaction was stirred at 0° C. for 30 minutes. 4-(7-Bromo-2-chloro-8-methyl-quinazolin-4-yl)-1,4-oxazepane (206 mg, 0.58 mmol) was added and the vial was sealed. The reaction was heated to 60° C. and stirred overnight. Water (10 mL) was added and the reaction was extracted with ethyl acetate (10 mL). The organics were washed with NaHCO₃, dried over Na₂SO₄ and concentrated in vacuo to yield 4-[7-bromo-2-(1,2,3,5,6,7-hexahydropyrrolizin-8-ylmethoxy)-8-methyl-quinazolin-4-yl]-1,4-oxazepane (245 mg, 0.53 mmol, 92% yield) as a pink solid.

UPLC-MS (ES⁺, Short acidic): 1.38 min, m/z 463.1 [M+H]⁺

¹H NMR (400 MHz, DMSO-d6) δ/ppm: 7.66 (d, J=9.0 Hz, 1H), 7.42 (d, J=9.1 Hz, 1H), 4.04 (s, 2H), 3.99-3.96 (m, 4H), 3.87-3.85 (m, 2H), 3.71 (t, J=5.3 Hz, 2H), 2.96-2.91 (m, 2H), 2.61 (s, 3H), 2.57-2.54 (m, 1H), 2.07-2.01 (m, 2H), 1.93-1.70 (m, 7H), 1.60-1.53 (m, 2H).

STEP C, 4-[2-(1,2,3,5,6,7-hexahydropyrrolizin-8-ylmethoxy)-7-[3-(methoxymethoxy)-1-naphthyl]-8-methyl-quinazolin-4-yl]-1,4-oxazepane: 4-[7-bromo-2-(1,2,3,5,6,7-hexahydropyrrolizin-8-ylmethoxy)-8-methyl-quinazolin-4-yl]-1,4-oxazepane (245 mg, 0.53 mmol), Cs₂CO₃ (519 mg, 1.59 mmol) and 2-(3-(2-methoxymethoxy)naphthalene-1-yl)-4,4,5,5-tetramethyl-1,3,2-dioxaborolane (200 mg, 0.64 mmol) in 1,4-dioxane (3 mL) and water (1 mL) were degassed for 30 minutes in a microwave vial. [1,1′-Bis(diphenylphosphino)ferrocene]dichloropalladium II (78 mg, 0.11 mmol) was added and the reaction was stirred at 95° C. overnight. The mixture was concentrated in vacuo and the crude was purified by column chromatography eluting 70% ethyl acetate in petroleum ether followed by 0-12% 1 N NH₃ MeOH in DCM. The desired fractions were concentrated in vacuo to yield 4-[2-(1,2,3,5,6,7-hexahydropyrrolizin-8-ylmethoxy)-7-[3-(methoxymethoxy)-1-naphthyl]-8-methyl-quinazolin-4-yl]-1,4-oxazepane (181 mg, 0.32 mmol, 60% yield) as a brown oil.

UPLC-MS (ES⁺, Short acidic): 1.49 min, m/z 569.3 [M+H]⁺.

¹H NMR (400 MHz, DMSO-d6) δ/ppm: 7.92 (d, J=8.2 Hz, 1H), 7.88 (d, J=8.5 Hz, 1H), 7.55 (s, 1H), 7.51-7.48 (m, 1H), 7.24 (d, J=7.5 Hz, 1H), 7.20 (d, J=8.5 Hz, 1H), 7.14 (s, 1H), 5.39-5.35 (m, 2H), 4.10 (br s, 2H), 3.86-3.80 (m, 2H), 3.72-3.70 (m, 2H), 3.66-3.62 (m, 2H), 3.46 (s, 3H), 2.99-2.94 (m, 2H), 2.60-2.56 (m, 2H), 2.21 (s, 3H), 1.97-1.89 (m, 2H), 1.83-1.57 (m, 18H).

STEP D, 4-[2-(1,2,3,5,6,7-hexahydropyrrolizin-8-ylmethoxy)-8-methyl-4-(1,4-oxazepan-4-yl) quinazolin-7-yl]naphthalen-2-ol: Triethylsilane (0.5 mL, 3.18 mmol) and 4-[2-(1,2,3,5,6,7-hexahydropyrrolizin-8-ylmethoxy)-7-[3-(methoxymethoxy)-1-naphthyl]-8-methyl-quinazolin-4-yl]-1,4-oxazepane (181 mg, 0.32 mmol) were combined in DCM (3 mL) at 0° C. Trifluoroacetic acid (2.4 mL, 31.83 mmol) was added and the reaction was stirred at 25° C. for 1 h. The mixture was concentrated in vacuo. The crude product was purified by revers phase column chromatography eluting 0-50% MeCN in H₂O (containing 0.1% formic acid). The desired fractions were taken up in a minimal amount of MeOH and loaded onto an SCX column. MeOH (20 ml) was passed though the column, then the product was washed off with 1 M NH₃ in MeOH and concentrated in vacuo to yield 4-[2-(1,2,3,5,6,7-hexahydropyrrolizin-8-ylmethoxy)-8-methyl-4-(1,4-oxazepan-4-yl)quinazolin-7-yl]naphthalen-2-ol (110 mg, 0.21 mmol, 66% yield) as a white solid.

UPLC-MS (ES⁺, Long acidic): 2.85 min, m/z 525.4 [M+H]⁺.

¹H NMR (400 MHz, DMSO-d6) δ/ppm: 9.90 (s, 1H), 7.95 (d, J=8.6 Hz, 1H), 7.79 (d, J=8.3 Hz, 1H), 7.43-7.39 (m, 1H), 7.21-7.18 (m, 3H), 7.10 (d, J=8.6 Hz, 1H), 6.95 (s, 1H), 4.07-4.04 (m, 6H), 3.92-3.90 (m, 2H), 3.77-3.74 (m, 2H), 2.97-2.92 (m, 2H), 2.58-2.56 (m, 1H), 2.18 (s, 3H), 2.12-2.06 (m, 2H), 1.96-1.90 (m, 2H), 1.85-1.73 (m, 4H), 1.61-1.55 (m, 2H) (2H hidden under solvent peak).

### Example 3

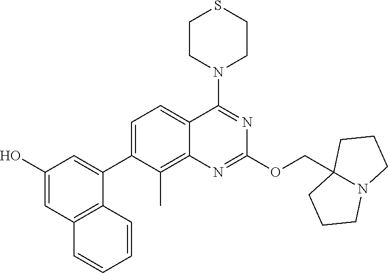 [SMILES: Cc1c(-c2cc(O)cc3ccccc23)ccc2c(N3CCSCC3)nc(OCC34CCCN3CCC4)nc12]

### 4-[2-(1,2,3,5,6,7-hexahydropyrrolizin-8-ylmethoxy)-8-methyl-4-thiomorpholino-quinazolin-7-yl]naphthalen-2-ol

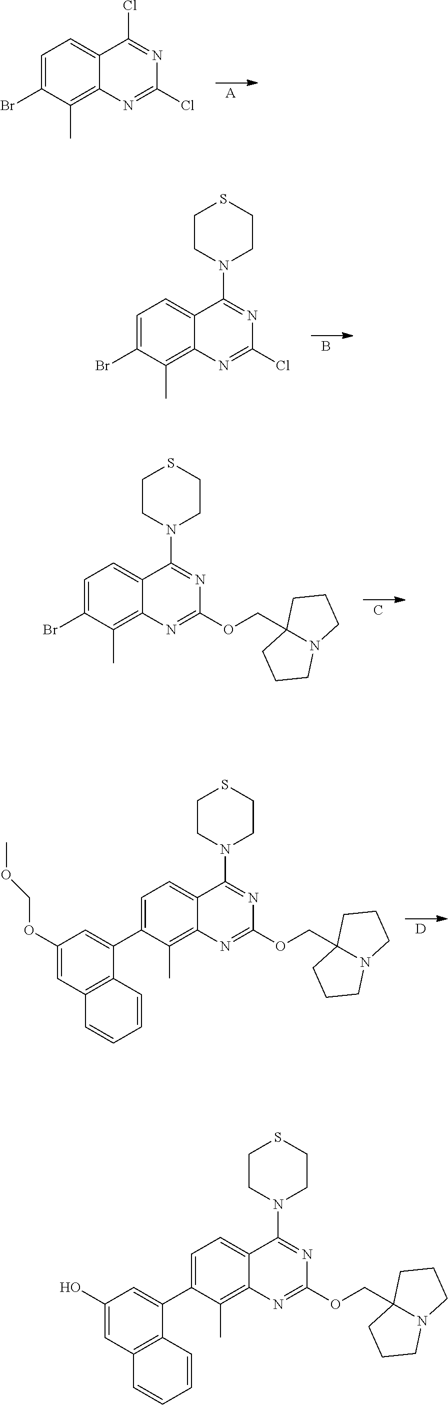

STEP A, 4-(7-bromo-2-chloro-8-methyl-quinazolin-4-yl)thiomorpholine: Thiomorpholine (0.14 mL, 1.37 mmol) was added to a stirred solution of 7-bromo-2,4-dichloro-8-methylquinazoline (200 mg, 0.69 mmol) in DCM (7 mL) at 0° C. and the reaction was left to stir at 0° C. for 3 h. The mixture was dry loaded onto silica and the product was purified by flash column chromatography eluting with 0-80% EtOAc in petroleum ether. The desired fractions were combined and concentrated under reduced pressure to afford 4-(7-bromo-2-chloro-8-methyl-quinazolin-4-yl)thiomorpholine (220 mg, 0.61 mmol, 89% yield) as a pale yellow solid.

UPLC-MS (ES⁺, Short acidic): 2.28 min, m/z 360.0 [M+H]⁺.

¹H NMR (400 MHz, DMSO-d6) δ/ppm: 7.75-7.67 (m, 2H), 4.04-3.99 (m, 4H), 2.87-2.83 (m, 4H), 2.63 (s, 3H).

STEP B, 4-[7-bromo-2-(1,2,3,5,6,7-hexahydropyrrolizin-8-ylmethoxy)-8-methyl-quinazolin-4-yl] thiomorpholine: sodium hydride, 60% dispersed in mineral oil (74 mg, 1.84 mmol) was added to a stirred solution of (tetrahydro-1H-pyrrolizin-7a(5H)-yl)methanol (173 mg, 1.23 mmol) in dry THF (6 mL) at 0° C. and the reaction was allowed to stir at 0° C. for 30 minutes. 4-(7-Bromo-2-chloro-8-methyl-quinazolin-4-yl)thiomorpholine (220 mg, 0.61 mmol) was added and the reaction was heated to 60° C. and stirred overnight. The reaction was cooled to room temperature and concentrated in vacuo. The residue was taken up in a mixture of EtOAc (100 mL) and water (100 mL). The organic phase was collected and the aqueous phase was washed with EtOAc (3×100 mL). The combined organics were washed with water (100 mL) and saturated sodium bicarbonate (100 mL). The organic layer was dried over Na₂SO₄, filtered and concentrated in vacuo to afford 4-[7-bromo-2-(1,2,3,5,6,7-hexahydropyrrolizin-8-ylmethoxy)-8-methyl-quinazolin-4-yl]thiomorpholine (275 mg, 0.59 mmol, 97% yield) as a soft amber solid.

UPLC-MS (ES⁺, Short acidic): 1.55 min, m/z 465.0 [M+H]⁺.

¹H NMR (400 MHz, DMSO-d6) δ/ppm: 7.62 (d, J=9.0 Hz, 1H), 7.47 (d, J=8.9 Hz, 1H), 4.05 (s, 2H), 3.93-3.88 (m, 4H), 2.97-2.90 (m, 2H), 2.86-2.80 (m, 4H), 2.62 (s, 3H), 1.94-1.69 (m, 7H), 1.61-1.52 (m, 2H), 0.89-0.71 (m, 2H).

STEP C, 4-[2-(1,2,3,5,6,7-hexahydropyrrolizin-8-ylmethoxy)-7-[3-(methoxymethoxy)-1-naphthyl]-8-methyl-quinazolin-4-yl]thiomorpholine: A suspension of 4-[7-bromo-2-(1,2,3,5,6,7-hexahydro pyrrolizin-8-ylmethoxy)-8-methyl-quinazolin-4-yl]thiomorpholine (78 mg, 0.17 mmol), 2-(3-(2-methoxymethoxy)naphthalene-1-yl)-4,4,5,5-tetramethyl-1,3,2-dioxaborolane (79 mg, 0.25 mmol) and Cs₂CO₃ (110 mg, 0.34 mmol) in a mixture of 1,4-dioxane (1.3 mL) and water (0.4 mL) was degassed with nitrogen for 15 minutes. [1,1′-Bis(di-tert-butylphosphino)ferrocene]dichloropalladium (II) (22 mg, 0.03 mmol) was added, the reaction was heated to 95° C. and stirred for 2 h. The reaction was cooled to room temperature and the solvent was removed in vacuo. The residue was taken up in DCM and dry loaded onto silica. The product was purified by flash column chromatography on silica gel eluting with 0-60% (20% 1M ammonia in MeOH) in DCM. The desired fractions were combined and concentrated in vacuo to afford 4-[2-(1,2,3,5,6,7-hexahydropyrrolizin-8-ylmethoxy)-7-[3-(methoxymethoxy)-1-naphthyl]-8-methyl-quinazolin-4-yl]thiomorpholine (39 mg, 0.07 mmol, 41% yield) as a light brown solid.

UPLC-MS (ES⁺, Short acidic): 1.86 min, m/z 571.3 [M+H]⁺.

¹H NMR (400 MHz, DMSO-d6) δ/ppm: 7.92 (d, J=8.3 Hz, 1H), 7.83 (d, J=8.5 Hz, 1H), 7.54 (d, J=2.4 Hz, 1H), 7.52-7.47 (m, 1H), 7.32-7.27 (m, 1H), 7.24-7.18 (m, 2H), 7.13 (d, J=2.3 Hz, 1H), 5.40-5.34 (m, 2H), 4.20 (br s, 2H), 4.03-3.94 (m, 4H), 3.46-3.45 (m, 3H), 3.21-2.95 (br s, 2H), 2.93-2.84 (m, 4H), 2.21 (s, 3H), 2.10-1.54 (m, 9H), 1.17 (d, J=13.5 Hz, 1H).

STEP D, 4-[2-(1,2,3,5,6,7-hexahydropyrrolizin-8-ylmethoxy)-8-methyl-4-thiomorpholino-quinazolin-7-yl]naphthalen-2-ol: Triethylsilane (0.11 mL, 0.69 mmol) was added to a solution of 4-[2-(1,2,3,5,6,7-hexahydropyrrolizin-8-ylmethoxy)-7-[3-(methoxymethoxy)-1-naphthyl]-8-methyl-quinazolin-4-yl] thiomorpholine (39 mg, 0.07 mmol) in DCM (0.7 mL) at 0° C. Trifluoroacetic acid (0.53 mL, 6.9 mmol) was added and the reaction was allowed to stir at room temperature for 1 h. The mixture was dry loaded onto silica and purified by reverse phase chromatography eluting with 5-50% MeCN (0.1% formic acid) in water (0.1% formic acid) with fractions containing product isolated by SCX (methanol wash (x2) followed by 1 M NH₃ in MeOH (x2)). The filtrate was concentrated in vacuo to afford 4-[2-(1,2,3,5,6,7-hexahydropyrrolizin-8-ylmethoxy)-8-methyl-4-thiomorpholino-quinazolin-7-yl]naphthalen-2-ol (34 mg, 0.06 mmol, 92% yield) as a yellow solid.

UPLC-MS (ES⁺, Long acidic): 3.31 min, m/z 527.4 [M+H]⁺.

¹H NMR (400 MHz, DMSO-d6) δ/ppm: 9.90 (s, 1H), 7.83-7.76 (m, 2H), 7.43-7.38 (m, 1H), 7.21 (d, J=2.4 Hz, 1H), 7.18-7.15 (m, 3H), 6.95 (d, J=2.4 Hz, 1H), 4.07 (s, 2H), 4.00-3.93 (m, 4H), 2.97-2.85 (m, 6H), 2.19 (s, 3H), 2.03-1.87 (m, 3H), 1.86-1.69 (m, 5H), 1.63-1.53 (m, 2H).

### Example 4

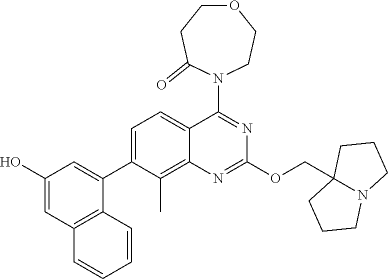 [SMILES: Cc1c(-c2cc(O)cc3ccccc23)ccc2c(N3CCOCCC3=O)nc(OCC34CCCN3CCC4)nc12]

### 4-[2-(1,2,3,5,6,7-hexahydropyrrolizin-8-ylmethoxy)-7-(3-hydroxy-1-naphthyl)-8-methyl-quinazolin-4-yl]-1,4-oxazepan-5-one

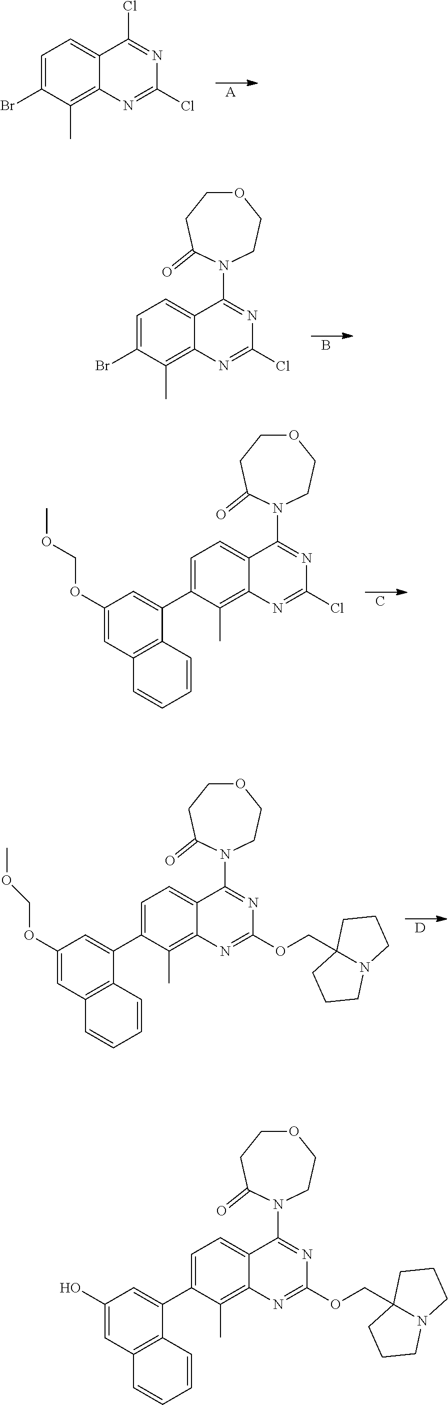

STEP A, 4-(7-bromo-2-chloro-8-methyl-quinazolin-4-yl)-1,4-oxazepan-5-one. Lithium bis(trimethylsilyl)amide (1.71 mL, 1.71 mmol) was added dropwise to a stirred solution of [1,4]-Oxazepan-5-one (197.17 mg, 1.71 mmol) in dry THF (18 mL) at 0° C. and the reaction was allowed to stir at this temperature for 30 minutes. 7-Bromo-2,4-dichloro-8-methylquinazoline (500.mg, 1.71 mmol) was added and the reaction continued to stir at 0° C. for 3 h. The reaction was concentrated to dryness. The residue was re-dissolved in DCM and dry loaded onto silica. The product was purified by flash column chromatography on silica gel (40 g) eluting with 0-100% EtOAc in Petroleum Ether. The desired fraction was concentrated to dryness to afford 4-(7-bromo-2-chloro-8-methyl-quinazolin-4-yl)-1,4-oxazepan-5-one (357.4 mg, 0.96 mmol, 56.3% yield) as a powdery white solid.

UPLC-MS (ES⁺, Short acidic): 1.88 min, m/z 372.0 [M+H]⁺.

¹H NMR (400 MHz, DMSO-d6) δ/ppm: 7.90 (d, J=9.0 Hz, 1H), 7.81 (dd, J=9.0, 0.6 Hz, 1H), 4.16-4.11 (m, 2H), 3.96-3.88 (m, 4H), 3.03-2.98 (m, 2H), 2.74 (s, 3H).

STEP B, 4-[2-chloro-7-[3-(methoxymethoxy)-1-naphthyl]-8-methyl-quinazolin-4-yl]-1,4-oxazepan-5-one. A suspension of 4-(7-bromo-2-chloro-8-methyl-quinazolin-4-yl)-1,4-oxazepan-5-one (357.4 mg, 0.96 mmol), 2-(3-(2-methoxymethoxy)naphthalene-1-yl)-4,4,5,5-tetramethyl-1,3,2-dioxaborolane (363.56 mg, 1.16 mmol) and Potassium carbonate (266.55 mg, 1.93 mmol) in a mixture of 1,4-Dioxane (8.85 mL) and Water (0.89 mL) was de-gassed with nitrogen for 10 minutes. [1,1′-Bis(di-tert-butylphosphino)ferrocene]dichloropalladium(II) (125.7 mg, 0.19 mmol) was added and the reaction was heated to 80° C. before stirring at this temperature for 1 h and 45 minutes. The reaction was cooled to room temperature and silica was added for dry loading. The product was purified by flash column chromatography on silica gel (40 g) eluting with 30-85% EtOAc in Petroleum Ether. The desired fractions were combined and concentrated under reduced pressure to afford 4-[2-chloro-7-[3-(methoxymethoxy)-1-naphthyl]-8-methyl-quinazolin-4-yl]-1,4-oxazepan-5-one (148 mg, 0.31 mmol, 32.1% yield) as a translucent oily residue.

UPLC-MS (ES⁺, Short acidic): 2.10 min, m/z 478.1 [M+H]⁺.

¹H NMR (400 MHz, DMSO-d6) δ/ppm: 8.00 (dd, J=8.6, 0.6 Hz, 1H), 7.95 (d, J=8.2 Hz, 1H), 7.62-7.58 (m, 2H), 7.54-7.49 (m, 1H), 7.34-7.29 (m, 1H), 7.21-7.17 (m, 2H), 5.41-5.36 (m, 2H), 4.21-4.16 (m, 2H), 3.99-3.93 (m, 4H), 3.46 (s, 3H), 3.06-3.01 (m, 2H), 2.34 (s, 3H).

STEP C, 4-[2-(1,2,3,5,6,7-hexahydropyrrolizin-8-ylmethoxy)-7-[3-(methoxymethoxy)-1-naphthyl]-8-methyl-quinazolin-4-yl]-1,4-oxazepan-5-one. A suspension of 4-[2-chloro-7-[3-(methoxymethoxy)-1-naphthyl]-8-methyl-quinazolin-4-yl]-1,4-oxazepan-5-one (148.mg, 0.31 mmol), 1,2,3,5,6,7-hexahydropyrrolizin-8-ylmethanol (218.mg, 1.55 mmol), Cesium carbonate (201.95 mg, 0.62 mmol) and 4,6-Bis(diphenylphosphino)-10H-phenoxazine (68.32 mg, 0.12 mmol) in dry 1,4-Dioxane (3 mL) was de-gassed with nitrogen for 10 minutes. Palladium (II) acetate (13.9 mg, 0.06 mmol) was added. The reaction was heated to 110° C. and allowed to stir at this temperature for 3 h. The reaction was cooled to room temperature and silica was added for dry loading. The product was purified by flash column chromatography on silica gel (40 g) eluting with 25-100% MeOH in DCM. The desired fractions were combined and concentrated under reduced pressure to afford 4-[2-(1,2,3,5,6,7-hexahydropyrrolizin-8-ylmethoxy)-7-[3-(methoxymethoxy)-1-naphthyl]-8-methyl-quinazolin-4-yl]-1,4-oxazepan-5-one (53.5 mg, 0.0918 mmol, 29.65% yield) as a clear translucent residue.

UPLC-MS (ES⁺, Short acidic): 1.70 min, m/z 583.3 [M+H]⁺

STEP D, 4-[2-(1,2,3,5,6,7-hexahydropyrrolizin-8-ylmethoxy)-7-(3-hydroxy-1-naphthyl)-8-methyl-quinazolin-4-yl]-1,4-oxazepan-5-one. 4-[2-(1,2,3,5,6,7-hexahydropyrrolizin-8-ylmethoxy)-7-[3-(methoxymethoxy)-1-naphthyl]-8-methyl-quinazolin-4-yl]-1,4-oxazepan-5-one (53.5 mg, 0.09 mmol) was dissolved in DCM (2 mL) and the reaction was cooled to 0° C. Triethylsilane (0.15 mL, 0.92 mmol) was added, followed by slow addition of Trifluoroacetic acid (0.21 mL, 2.75 mmol). The reaction was stirred at 0 degrees for 5 minutes and the reaction was then warmed to room temperature. The reaction was allowed to stir at 25° C. for 45 minutes. Celite was added to the reaction and it was concentrated to dryness. The product was purified by reverse phase chromatography (25 g) eluting with 0-40% MeCN (0.1% formic acid) in Water (0.1% formic acid) with fractions containing product isolated by SCX (methanol wash (x2) followed by 1 M NH₃ in MeOH (x2)). The filtrate was concentrated under reduced pressure to afford 4-[2-(1,2,3,5,6,7-hexahydropyrrolizin-8-ylmethoxy)-7-(3-hydroxy-1-naphthyl)-8-methyl-quinazolin-4-yl]-1,4-oxazepan-5-one (11.8 mg, 0.02 mmol, 23.8% yield) as a pale yellow solid.

UPLC-MS (ES⁺, Long acidic): 3.11 min, m/z 539.8 [M+H]⁺.

¹H NMR (400 MHz, DMSO-d6) δ/ppm: 9.95 (s, 1H), 7.85 (dd, J=8.6, 0.6 Hz, 1H), 7.80 (d, J=8.3 Hz, 1H), 7.44-7.39 (m, 1H), 7.32 (d, J=8.6 Hz, 1H), 7.23 (d, J=2.4 Hz, 1H), 7.21-7.16 (m, 1H), 7.15-7.11 (m, 1H), 6.97 (d, J=2.4 Hz, 1H), 4.20-4.14 (m, 2H), 4.14-4.09 (m, 2H), 3.98-3.91 (m, 4H), 3.03-2.91 (m, 2H), 2.29 (s, 3H), 1.99-1.89 (m, 3H), 1.89-1.68 (m, 6H), 1.66-1.57 (m, 3H).

### Example 5

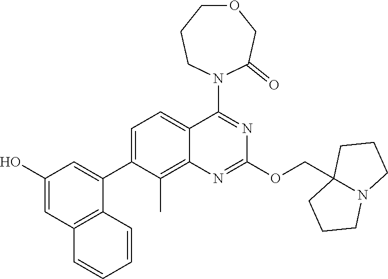 [SMILES: Cc1c(-c2cc(O)cc3ccccc23)ccc2c(N3CCCOCC3=O)nc(OCC34CCCN3CCC4)nc12]

### 4-[2-(1,2,3,5,6,7-hexahydropyrrolizin-8-ylmethoxy)-7-(3-hydroxy-1-naphthyl)-8-methyl-quinazolin-4-yl]-1,4-oxazepan-3-one

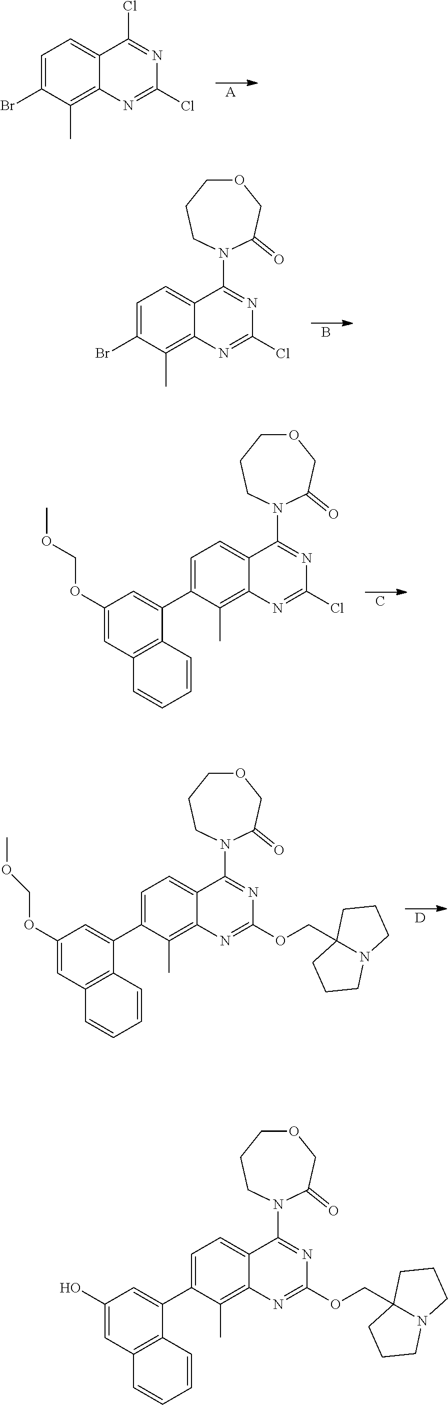

STEP A, 4-(7-bromo-2-chloro-8-methyl-quinazolin-4-yl)-1,4-oxazepan-3-one. Lithium bis(trimethylsilyl)amide (1.71 mL, 1.71 mmol) was added dropwise to a stirred solution of [1,4]-oxazepan-3-one (197.17 mg, 1.71 mmol) in dry THF (18 mL) at 0° C. and the reaction was allowed to stir at this temperature for 30 minutes. 7-Bromo-2,4-dichloro-8-methylquinazoline (500.mg, 1.71 mmol) was added and the reaction continued to stir at 0° C. for 3 h. The reaction was concentrated to dryness. The residue was re-dissolved in DCM and dry loaded onto silica. The product was purified by flash column chromatography on silica gel (40 g) eluting with 0-100% EtOAc in Petroleum Ether. The desired fraction was concentrated to dryness to afford 4-(7-bromo-2-chloro-8-methyl-quinazolin-4-yl)-1,4-oxazepan-3-one (273.8 mg, 0.7387 mmol, 43.1% yield) as a powdery white solid.

UPLC-MS (ES⁺, Short acidic): 1.93 min, m/z 372.0 [M+H]⁺.

¹H NMR (400 MHz, DMSO-d6) δ/ppm: 7.95 (d, J=9.0 Hz, 1H), 7.68 (dd, J=9.1, 0.6 Hz, 1H), 4.46 (s, 2H), 4.20.4.15 (m, 2H), 4.00-3.94 (m, 2H), 2.74 (s, 3H), 2.06-1.99 (m, 2H).

STEP B, 4-[2-chloro-7-[3-(methoxymethoxy)-1-naphthyl]-8-methyl-quinazolin-4-yl]-1,4-oxazepan-3-one. A suspension of 4-(7-bromo-2-chloro-8-methyl-quinazolin-4-yl)-1,4-oxazepan-3-one (673.6 mg, 1.82 mmol), 2-(3-(2-methoxymethoxy)naphthalene-1-yl)-4,4,5,5-tetramethyl-1,3,2-dioxaborolane (685.21 mg, 2.18 mmol) and Potassium carbonate (502.38 mg, 3.63 mmol) in a mixture of 1,4-Dioxane (16.7 mL) and Water (1.67 mL) was de-gassed with nitrogen for 20 minutes. [1,1′-Bis(di-tert-butylphosphino)ferrocene]dichloropalladium(II) (236.9 mg, 0.36 mmol) was added and the reaction was heated to 80° C. before stirring at this temperature for 2 hours and 10 minutes. The reaction was cooled to room temperature and silica was added for dry loading. The product was purified by flash column chromatography on silica gel (40 g) eluting with 30-85% EtOAc in Petroleum Ether. The desired fractions were combined and concentrated under reduced pressure to afford 4-[2-chloro-7-[3-(methoxymethoxy)-1-naphthyl]-8-methyl-quinazolin-4-yl]-1,4-oxazepan-3-one (351.3 mg, 0.73 mmol, 40.4% yield) as a clear oily residue.

UPLC-MS (ES⁺, Short acidic): 2.17 min, m/z 478.1 [M+H]⁺.

¹H NMR (400 MHz, DMSO-d6) δ/ppm: 7.95 (d, J=8.4 Hz, 1H), 7.88 (dd, J=8.6, 0.6 Hz, 1H), 7.65 (d, J=8.6 Hz, 1H), 7.59 (d, J=2.5 Hz, 1H), 7.54-7.49 (m, 1H), 7.34-7.29 (m, 1H), 7.21-7.17 (m, 2H), 5.41-5.35 (m, 2H), 4.49 (s, 2H), 4.25-4.19 (m, 2H), 4.02-3.97 (m, 2H), 3.46 (s, 3H), 2.35-2.31 (m, 3H), 2.12-2.03 (m, 2H).

STEP C, 4-[2-(1,2,3,5,6,7-hexahydropyrrolizin-8-ylmethoxy)-7-[3-(methoxymethoxy)-1-naphthyl]-8-methyl-quinazolin-4-yl]-1,4-oxazepan-3-one. A suspension of 4-[2-chloro-7-[3-(methoxymethoxy)-1-naphthyl]-8-methyl-quinazolin-4-yl]-1,4-oxazepan-3-one (351.3 mg, 0.74 mmol), 1,2,3,5,6,7-hexahydropyrrolizin-8-ylmethanol (518.97 mg, 3.68 mmol), Cesium carbonate (479.36 mg, 1.47 mmol) and 4,6-Bis(diphenylphosphino)-10H-phenoxazine (162.16 mg, 0.29 mmol) in dry 1,4-Dioxane (7.5 mL) was de-gassed with nitrogen for 10 minutes. Palladium (II) acetate (33.mg, 0.1500 mmol) was added. The reaction was heated to 110° C. and allowed to stir at this temperature for 3 h. The reaction was cooled to room temperature and silica was added for dry loading. The product was purified by flash column chromatography on silica gel (40 g) eluting first with 20-100% EtOAc in Petroleum ether followed by 0-20% (1M ammonia in MeOH) in DCM. The desired fractions were combined and concentrated under reduced pressure to afford 4-[2-(1,2,3,5,6,7-hexahydropyrrolizin-8-ylmethoxy)-7-[3-(methoxymethoxy)-1-naphthyl]-8-methyl-quinazolin-4-yl]-1,4-oxazepan-3-one (37.1 mg, 0.064 mmol, 8.6% yield) as a translucent residue.

UPLC-MS (ES⁺, Short acidic): 1.67 min, m/z 583.6 [M+H]⁺.

STEP D, 4-[2-(1,2,3,5,6,7-hexahydropyrrolizin-8-ylmethoxy)-7-(3-hydroxy-1-naphthyl)-8-methyl-quinazolin-4-yl]-1,4-oxazepan-3-one. 4-[2-(1,2,3,5,6,7-hexahydropyrrolizin-8-ylmethoxy)-7-[3-(methoxymethoxy)-1-naphthyl]-8-methyl-quinazolin-4-yl]-1,4-oxazepan-3-one (37.1 mg, 0.06 mmol) was dissolved in DCM (2.5 mL) and the reaction was cooled to 0° C. Triethylsilane (0.1 mL, 0.64 mmol) was added, followed by slow addition of Trifluoroacetic acid (0.15 mL, 1.91 mmol). The reaction was stirred at 0° C. for 5 minutes and the reaction was then warmed to room temperature. The reaction was allowed to stir at 25° C. for 2 h and 40 minutes. Celite was added to the reaction and it was concentrated to dryness. The product was purified by reverse phase chromatography (12 g) eluting with 0-40% MeCN (0.1% formic acid) in Water (0.1% formic acid) with fractions containing product isolated by SCX (methanol wash (x2) followed by 1 M NH₃ in MeOH (x2)). The filtrate was concentrated to dryness to afford 4-[2-(1,2,3,5,6,7-hexahydropyrrolizin-8-ylmethoxy)-7-(3-hydroxy-1-naphthyl)-8-methyl-quinazolin-4-yl]-1,4-oxazepan-3-one (8.1 mg, 0.015 mmol, 23.6% yield) as a pale yellow powdery solid.

UPLC-MS (ES⁺, Long acidic): 3.23 min, m/z 539.7 [M+H]⁺.

¹H NMR (400 MHz, DMSO-d6) δ/ppm: 9.95 (s, 1H), 7.80 (d, J=8.3 Hz, 1H), 7.75 (dd, J=8.5, 0.5 Hz, 1H), 7.44-7.39 (m, 1H), 7.35 (d, J=8.6 Hz, 1H), 7.23 (d, J=2.4 Hz, 1H), 7.21-7.16 (m, 1H), 7.15-7.11 (m, 1H), 6.97 (d, J=2.4 Hz, 1H), 4.46 (s, 2H), 4.19-4.12 (m, 4H), 4.02-3.96 (m, 2H), 2.99-2.92 (m, 2H), 2.29 (s, 3H), 2.11 (m, 2H), 2.00-1.89 (m, 3H), 1.89-1.72 (m, 5H), 1.66-1.57 (m, 2H).

### Example 6

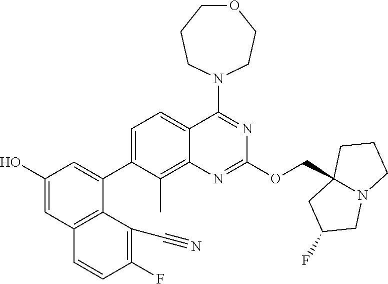 [SMILES: Cc1c(-c2cc(O)cc3ccc(F)c(C#N)c23)ccc2c(N3CCCOCC3)nc(OC[C@@]34CCCN3C[C@H](F)C4)nc12]

### rac-5-ethynyl-6-fluoro-4-[2-[[(2R,8S)-2-fluoro-1,2,3,5,6,7-hexahydropyrrolizin-8-yl]methoxy]-8-methyl-4-(1,4-oxazepan-4-yl)quinazolin-7-yl]naphthalen-2-ol

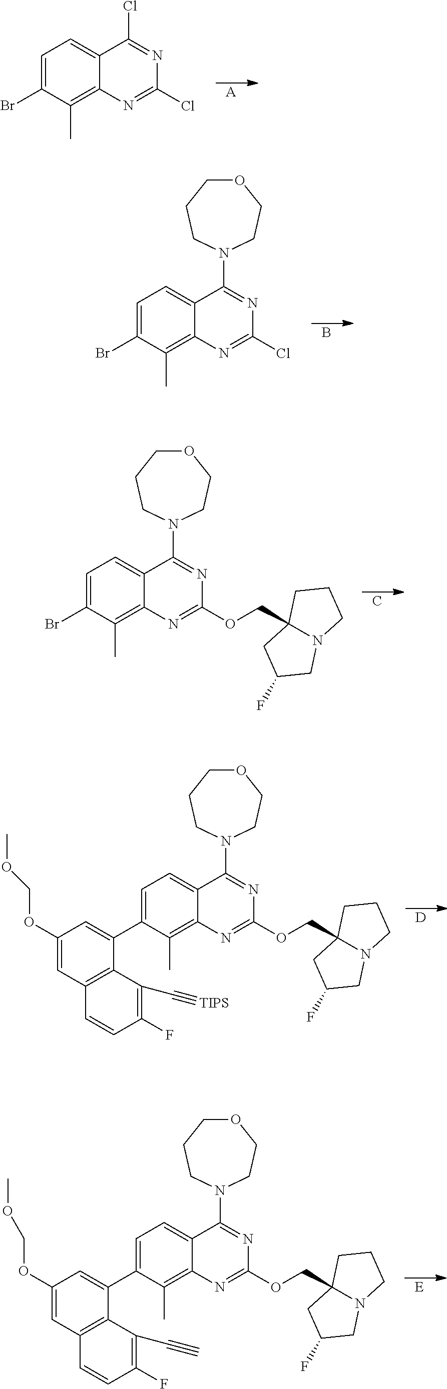

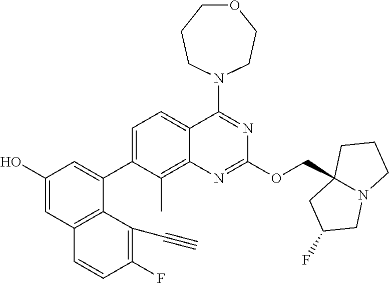 [SMILES: C#Cc1c(F)ccc2cc(O)cc(-c3ccc4c(N5CCCOCC5)nc(OC[C@@]56CCCN5C[C@H](F)C6)nc4c3C)c12]

2-[2-fluoro-6-(methoxymethoxy)-8-(4,4,5,5-tetramethyl-1,3,2-dioxaborolan-2-yl)-1-naphthyl]ethynyl-triisopropyl-silane used in STEP C was prepared following the procedure published in The Journal of Medicinal Chemistry: https://doi.org/10.1021/acs.jmedchem.1c01688

STEP A, 4-(7-bromo-2-chloro-8-methyl-quinazolin-4-yl)-1,4-oxazepane. This procedure is described in example 2 (STEP A).

STEP B, rac-4-[7-bromo-2-[[(2R,8S)-2-fluoro-1,2,3,5,6,7-hexahydropyrrolizin-8-yl]methoxy]-8-methyl-quinazolin-4-yl]-1,4-oxazepane. Sodium hydride, (60% dispersed in mineral oil) (29.61 mg, 0.74 mmol) was added to a stirred solution of rac-[(2R,8S)-2-fluoro-1,2,3,5,6,7-hexahydropyrrolizin-8-yl]methanol (78.56 mg, 0.4900 mmol) in dry THF (6 mL) at 0° C. and the reaction was allowed to stir at 0° C. for 30 minutes. 4-(7-bromo-2-chloro-8-methyl-quinazolin-4-yl)-1,4-oxazepane (88.mg, 0.25 mmol) was added and the reaction was heated to 60° C. before being allowed to stir overnight. The reaction was cooled to room temperature and concentrated to dryness. The residue was dissolved in a mixture of DCM and MeOH and silica was added for dry loading. The product was purified by flash column chromatography on silica gel (25 g) eluting first with 0-100% EtOAc in Petroleum Ether followed by 0-20% MeOH (1M NH₃) in DCM. The desired fractions were united and concentrated under reduced pressure to afford rac-4-[7-bromo-2-[[(2R,8S)-2-fluoro-1,2,3,5,6,7-hexahydropyrrolizin-8-yl]methoxy]-8-methyl-quinazolin-4-yl]-1,4-oxazepane (99.4 mg, 0.2073 mmol, 84.048% yield) as a brown residue.

UPLC-MS (ES⁺, Short acidic): 1.46 min, m/z 481.1 [M+H]⁺.

STEP C, rac-2-[2-fluoro-8-[2-[[(2R,8S)-2-fluoro-1,2,3,5,6,7-hexahydropyrrolizin-8-yl]methoxy]-8-methyl-4-(1,4-oxazepan-4-yl)quinazolin-7-yl]-6-(methoxymethoxy)-1-naphthyl]ethynyl-triisopropyl-silane. A suspension of 2-[2-fluoro-6-(methoxymethoxy)-8-(4,4,5,5-tetramethyl-1,3,2-dioxaborolan-2-yl)-1-naphthyl]ethynyl-triisopropyl-silane (337.26 mg, 0.66 mmol), rac-4-[7-bromo-2-[[(2R,8S)-2-fluoro-1,2,3,5,6,7-hexahydropyrrolizin-8-yl]methoxy]-8-methyl-quinazolin-4-yl]-1,4-oxazepane (210.3 mg, 0.44 mmol) and cesium carbonate (285.86 mg, 0.88 mmol) in a mixture of 1,4-Dioxane (8.23 mL) and Water (2.7438 mL) was de-gassed with nitrogen for 15 minutes. [1,1′-Bis(di-tert-butylphosphino)ferrocene]dichloropalladium(II) (28.59 mg, 0.04 mmol) was added and the reaction was heated to 90° C. The reaction was allowed to stir overnight. The reaction was cooled to room temperature and was filtered through a plug of Celite (washed twice with MeOH). The filtrate was concentrated to dryness. The resulting residue was taken up in a mixture of MeOH and DCM, and silica was added for dry loading. The product was purified by flash column chromatrography on silica gel (40 g) eluting with 0-20% (1M ammonia in MeOH) in DCM. The desired fractions were combined and concentrated under reduced pressure to afford rac-2-[2-fluoro-8-[2-[[(2R,8S)-2-fluoro-1,2,3,5,6,7-hexahydropyrrolizin-8-yl]methoxy]-8-methyl-4-(1,4-oxazepan-4-yl)quinazolin-7-yl]-6-(methoxymethoxy)-1-naphthyl]ethynyl-triisopropyl-silane (258.8 mg, 0.32 mmol, 75.1% yield) as a tan residue.

UPLC-MS (ES⁺, Short acidic): 2.20 min, m/z 785.5 [M+H]⁺.

¹H NMR (400 MHz, DMSO-d6) δ/ppm: 8.09 (m, 1H), 7.82 (d, J=8.8 Hz, 1H), 7.64 (d, J=2.7 Hz, 1H), 7.52 (t, J=8.9 Hz, 1H), 7.10 (d, J=8.7 Hz, 1H), 7.03 (d, J=2.6 Hz, 1H), 5.37-5.32 (m, 2H), 4.18-3.95 (m, 6H), 3.92 (s, 3H), 3.83-3.71 (m, 2H), 3.43 (s, 3H), 3.18-2.97 (m, 3H), 2.90-2.79 (m, 1H), 2.56-2.44 (m, 2H), 2.22 (d, J=3.5 Hz, 3H), 2.19-1.97 (m, 6H), 1.92-1.68 (m, 3H), 0.83-0.77 (m, 18H), 0.50-0.39 (m, 3H)

STEP D, rac-4-[7-[8-ethynyl-7-fluoro-3-(methoxymethoxy)-1-naphthyl]-2-[[(2R,8S)-2-fluoro-1,2,3,5,6,7-hexahydropyrrolizin-8-yl]methoxy]-8-methyl-quinazolin-4-yl]-1,4-oxazepane. Tetrabutylammonium fluoride 1.OM in THF (0.35 mL, 0.35 mmol) was added to a stirred solution of rac-2-[2-fluoro-8-[2-[[(2R,8S)-2-fluoro-1,2,3,5,6,7-hexahydropyrrolizin-8-yl]methoxy]-8-methyl-4-(1,4-oxazepan-4-yl)quinazolin-7-yl]-6-(methoxymethoxy)-1-naphthyl]ethynyl-triisopropyl-silane (249.8 mg, 0.32 mmol) in THF (6 mL) at 0° C. The reaction was allowed to warm to room temperature before stirring for 45 minutes. Silica was added for dry loading. The product was purified by flash column chromatography on silica gel (25 g) eluting with 0-20% MeOH (1M NH₃) in DCM. The desired fractions were combined and concentrated in vacuo to afford rac-4-[7-[8-ethynyl-7-fluoro-3-(methoxymethoxy)-1-naphthyl]-2-[[(2R,8S)-2-fluoro-1,2,3,5,6,7-hexahydropyrrolizin-8-yl]methoxy]-8-methyl-quinazolin-4-yl]-1,4-oxazepane (194.5 mg, 0.31 mmol, 97.2% yield) as a tan waxy residue.

UPLC-MS (ES⁺, Short acidic): 1.59 min, m/z 629.3 [M+H]⁺

¹H NMR (400 MHz, DMSO-d6) δ/ppm: 8.08-8.02 (m, 1H), 7.81 (d, J=8.7 Hz, 1H), 7.64 (d, J=2.6 Hz, 1H), 7.49 (t, J=9.0 Hz, 1H), 7.12-7.07 (m, 2H), 5.37-5.32 (m, 2H), 4.24-4.13 (m, 1H), 4.12-3.84 (m, 8H), 3.80-3.76 (m, 4H), 3.65 (s, 1H), 3.45 (s, 3H), 3.25-2.99 (m, 2H), 2.95-2.80 (m, 1H), 2.15 (s, 3H), 2.13-2.00 (m, 4H), 1.95-1.74 (m, 3H).

STEP E, rac-5-ethynyl-6-fluoro-4-[2-[[(2R,8S)-2-fluoro-1,2,3,5,6,7-hexahydropyrrolizin-8-yl]methoxy]-8-methyl-4-(1,4-oxazepan-4-yl)quinazolin-7-yl]naphthalen-2-ol. Hydrogen Chloride (5.mL, 5 mmol) in dioxane (1M) was slowly added to a solution of rac-4-[7-[8-ethynyl-7-fluoro-3-(methoxymethoxy)-1-naphthyl]-2-[[(2R,8S)-2-fluoro-1,2,3,5,6,7-hexahydropyrrolizin-8-yl]methoxy]-8-methyl-quinazolin-4-yl]-1,4-oxazepane (70 mg, 0.11 mmol) in dry 1,4-Dioxane (5 mL) at room temperature and was allowed to stir for 15 minutes. The reaction was concentrated to dryness and the resultant residue was taken up in DCM. Celite was added for dry loading. The product was purified by reversed phase chromatography eluting with 0-80% MeCN (0.1% ammonium carbonate) in Water (0.1% ammonium carbonate). The product was isolated by SCX (methanol wash (x2) followed by 1 M NH₃ in MeOH (x2)) from the desired fractions and the solvent was evaporated under reduced pressure to afford a pale yellow residue. The product was purified by preparative HPLC. The product was isolated by SCX (methanol wash (x2) followed by 1 M NH₃ in MeOH (x2)) from the desired fractions and the solvent was evaporated under reduced pressure to afford rac-5-ethynyl-6-fluoro-4-[2-[[(2R,8S)-2-fluoro-1,2,3,5,6,7-hexahydropyrrolizin-8-yl]methoxy]-8-methyl-4-(1,4-oxazepan-4-yl)quinazolin-7-yl]naphthalen-2-ol (8.4 mg, 0.014 mmol, 12.9% yield) as a powdery white solid analysed as a mixture of atropisomers.

UPLC-MS (ES⁺, Long acidic): 3.17 min, m/z 585.3 [M+H]⁺, 3.18 min, m/z 585.3 [M+H]⁺.

¹H NMR (400 MHz, DMSO-d6) δ/ppm: 10.04 (s, 1H), 7.96-7.91 (m, 1H), 7.80 (d, J=8.6 Hz, 1H), 7.45-7.39 (m, 1H), 7.30 (d, J=2.6 Hz, 1H), 7.06 (d, J=8.6 Hz, 1H), 6.92 (d, J=2.5 Hz, 1H), 5.37-5.19 (m, 1H), 4.16-4.09 (m, 1H), 4.09-3.94 (m, 6H), 3.93-3.88 (m, 2H), 3.79-3.73 (m, 2H), 3.72-3.70 (m, 1H), 3.15-2.99 (m, 3H), 2.88-2.78 (m, 1H), 2.17-2.00 (m, 7H), 1.89-1.71 (m, 3H).

The following examples in Table 3 were prepared using the same sequence of synthetic steps as for example 2, replacing 1,4-oxazepane in Step A with the corresponding building block described in Table 3.

| Building | | | | |
|---|---|---|---|---|
| block | Example | Structure | ¹HNMR | LC-MS |
| 4H,5H,6H,7H- pyra- zolo[1,5- a]pyrazine | 7 4-[4-(6,7-dihydro- 4H-pyrazolo[1,5- alpyrazin-5-yl)-2- (1,2,3,5,6,7- hexahydro- pyrrolizin- 8-ylmethoxy)-8- methyl-quinazolin- 7-yl]naphthalen-2- ol | 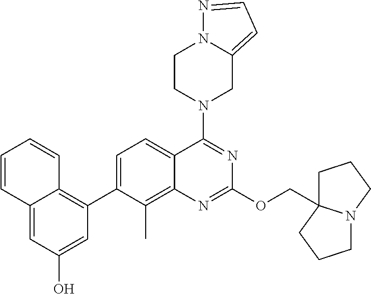 [SMILES: Cc1c(-c2cc(O)cc3ccccc23)ccc2c(N3CCn4nccc4C3)nc(OCC34CCCN3CCC4)nc12] | (400 MHz, DMSO-d₆) δ/ppm 9.91 (s, 1H), 7.98 (d, J = 8.4 Hz, 1H), 7.80 (d, J = 8.3 Hz, 1H), 7.48 (d, J = 1.8 Hz, 1H), 7.45-7.39 (m, 1H), 7.24-7.17 (m, 4H), 6.97 (d, J = 2.4 Hz, 1H), 6.22 (d, J = 1.8 Hz, 1H), 4.98 (s, 2H), 4.47 (dd, J = 5.4, 5.4 Hz, 2H), 4.27-4.17 (m, 2H), 4.10 (s, 2H), 2.98- 2.91 (m, 2H), 2.57- 2.52 (m, 2H), 2.22 (s, 3H), 1.99-1.73 (m, 6H), 1.63-1.56 (m, 2H). | ES⁺, (Method 1): 3.2 min, 547.4 [M + H]⁺ |
| 5H,6H,7H,8H- imidazo[1,5- a]pyrazine | 8 4-[4-(6,8-dihydro- 5H-imidazo[1,5- a]pyrazin-7-yl)-2- (1,2,3,5,6,7- hexahydro- pyrrolizin-8- ylmethoxy)-8- methyl-quinazolin- 7-yl]naphthalen-2- ol | 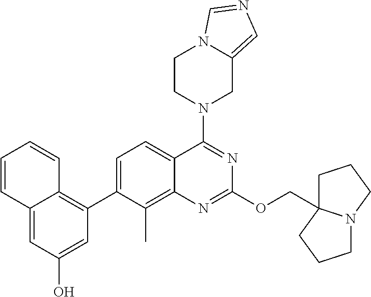 [SMILES: Cc1c(-c2cc(O)cc3ccccc23)ccc2c(N3CCn4cncc4C3)nc(OCC34CCCN3CCC4)nc12] | (400 MHz, DMSO-d₆) δ/ppm 9.91 (s, 1H), 7.96 (d, J = 8.4 Hz, 1H), 7.79 (d, J = 8.4 Hz, 1H), 7.68 (d, J = 0.9 Hz, 1H), 7.44-7.39 (m, 1H), 7.23-7.17 (m, 4H), 6.97 (d, J = 2.4 Hz, 1H), 6.84 (d, J = 1.0 Hz, 1H), 4.96 (s, 2H), 4.39 (dd, J = 5.4, 5.4 Hz, 2H), 4.14-4.08 (m, 4H), 2.98-2.91 (m, 2H), 2.57-2.52 (m, 2H), 2.21 (s, 3H), 1.98-1.73 (m, 6H), 1.64-1.55 (m, 2H). | ES⁺, (Method 1): 2.6 min, 547.3 [M + H]⁺ |
| 5H,6H,7H,8H- imidazo[1,2- a]pyrazine | 9 4-[4-(6,8-dihydro- 5H-imidazo[1,2- a]pyrazin-7-yl)-2- (1,2,3,5,6,7- hexahydro- pyrrolizin-8- ylmethoxy)-8- methyl-quinazolin- 7-yl]naphthalen-2- ol | 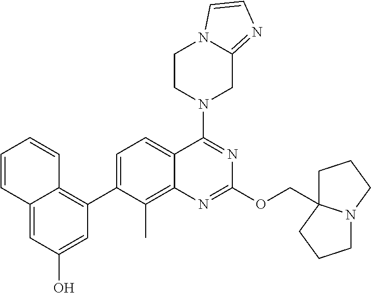 [SMILES: Cc1c(-c2cc(O)cc3ccccc23)ccc2c(N3CCn4ccnc4C3)nc(OCC34CCCN3CCC4)nc12] | (400 MHz, DMSO-d₆) δ/ppm 9.92 (s, 1H), 7.99 (d, J = 8.6 Hz, 1H), 7.80 (d, J = 8.4 Hz, 1H), 7.44-7.39 (m, 1H), 7.25-7.17 (m, 5H), 6.98 (d, J = 2.4 Hz, 1H), 6.94 (d, J = 1.2 Hz, 1H), 4.93 (s, 2H), 4.37- 4.32 (m, 2H), 4.25- 4.15 (m, 4H), 2.99 (s, 1H), 2.69-2.66 (m, 1H), 2.53 (d, J = 2.0 Hz, 1H), 2.47-2.41 (m, 2H), 2.35-2.32 (m, 1H), 2.22 (s, 3H), 2.01- 1.96 (m, 2H), 1.84- 1.78 (m, 2H), 1.70- 1.62 (m, 2H). | ES⁺, (Method 1): 2.6 min, 547.4 [M + H]⁺ |
| 1H,4H,5H,6H,7H- pyrazolo[4,3- c]pyridine | 10 4-[2-(1,2,3,5,6,7- hexahydro- pyrrolizin-8- ylmethoxy)-8- methyl-4-(1,4,6,7- tetrahydro- pyrazolo[4,3- c]pyridin-5- yl)quinazolin-7- yl]naphthalen-2-ol | 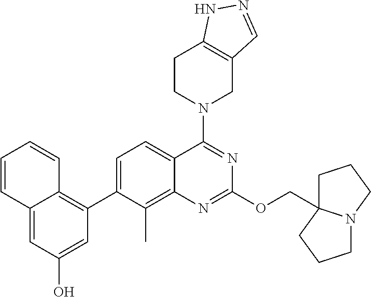 [SMILES: Cc1c(-c2cc(O)cc3ccccc23)ccc2c(N3CCc4[nH]ncc4C3)nc(OCC34CCCN3CCC4)nc12] | (400 MHz, DMSO-d₆) δ/ppm 9.90 (s, 1H), 7.92 (d, J = 8.4 Hz, 1H), 7.79 (d, J = 8.4 Hz, 1H), 7.44-7.39 (m, 1H), 7.23-7.17 (m, 4H), 6.97 (d, J = 2.4 Hz, 1H), 4.81-4.79 (m, 2H), 4.09 (s, 4H), 3.05 (d, J = 5.5 Hz, 2H), 2.98- 2.91 (m, 2H), 2.68- 2.52 (m, 2H), 2.46- 2.44 (m, 1H), 2.35- 2.32 (m, 1H), 2.19 (s, 3H), 1.98-1.72 (m, 6H), 1.63-1.55 (m, 2H). | ES⁺, (Method 1): 3.0 min, 547.3 [M + H]⁺ |
| 1H,4H,5H,6H,7H- pyrazolo[3,4- c]pyridine | 11 4-[2-(1,2,3,5,6,7- hexahydro- pyrrolizin-8- ylmethoxy)-8- methyl-4-(1,4,5,7- tetrahydro- pyrazolo[3,4- c]pyridin-6- yl)quinazolin-7- yl]naphthalen-2-ol | 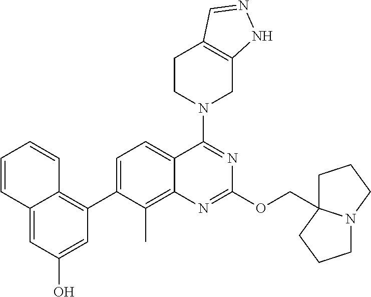 [SMILES: Cc1c(-c2cc(O)cc3ccccc23)ccc2c(N3CCc4cn[nH]c4C3)nc(OCC34CCCN3CCC4)nc12] | (400 MHz, DMSO-d₆) δ/ppm 12.7 (1H, s), 10.2 (1H, s) 9.94 (1H, s), 8.00-7.96 (1H, m), 7.80 (1H, d, J = 8.4 Hz), 7.45-7.40 (1H, m), 7.28-7.16 (4H, m), 6.97 (1H, d, J = 2.3 Hz), 4.89 (2H, s), 4.60- 4.58 (2H, m), 4.04- 4.01 (1H, m), 3.52- 3.47 (1H, m), 3.27 (2H, s), 2.99-2.96 (1H, m), 2.69-2.66 (1H, m), 2.47-2.45 (3H, m), 2.35-2.32 (2H, m), 2.24-2.01 (8H, m). | ES⁺, (Method 1): 3.1 min, 547.3 [M + H]⁺ |

### Example 12

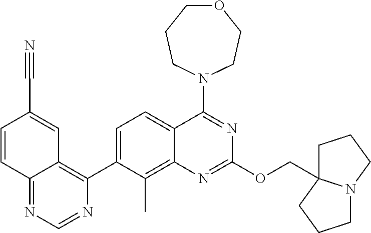 [SMILES: Cc1c(-c2ncnc3ccc(C#N)cc23)ccc2c(N3CCCOCC3)nc(OCC34CCCN3CCC4)nc12]

### 4-[2-(1,2,3,5,6,7-hexahydropyrrolizin-8-ylmethoxy)-8-methyl-4-(1,4-oxazepan-4-yl)quinazolin-7-yl]quinazoline-6-carbonitrile

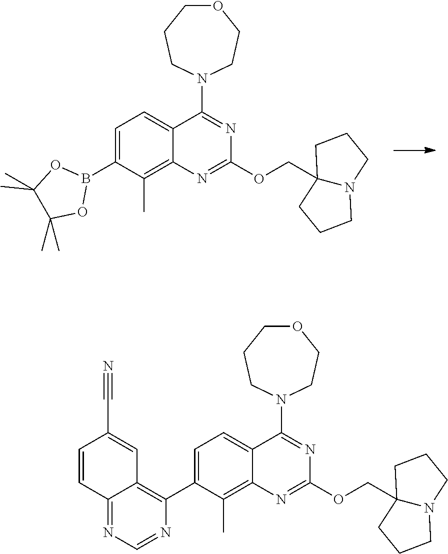 [SMILES: Cc1c(B2OC(C)(C)C(C)(C)O2)ccc2c(N3CCCOCC3)nc(OCC34CCCN3CCC4)nc12 ; Cc1c(-c2ncnc3ccc(C#N)cc23)ccc2c(N3CCCOCC3)nc(OCC34CCCN3CCC4)nc12]

To a degassed suspension of 4-[2-(1,2,3,5,6,7-hexahydropyrrolizin-8-ylmethoxy)-8-methyl-7-(4,4,5,5-tetramethyl-1,3,2-dioxaborolan-2-yl)quinazolin-4-yl]-1,4-oxazepane (120 mg, 0.24 mmol), 4-chloro-6-quinazolinecarbonitrile (67.12 mg, 0.35 mmol) and cesium carbonate (230.69 mg, 0.71 mmol) in 1,4-dioxane (3 mL) and Water (0.3 mL) was added [1,1′-bis(di-tert-butylphosphino)ferrocene]dichloropalladium (II) (15.38 mg, 0.02 mmol). The mixture was stirred at 95° C. overnight. The crude was diluted with EtOAc and filtered before concentration under vacuum. The crude was purified via flash column chromatography (5 g, KP-Amino D) eluting with 10-100% EtOAc in pet ether, followed by 0-20% MeOH in DCM. The product was further purifiedusing preparative HPLC. The fractions were collected and passed through an SCX column (1M NH₃ in MeOH). The concentrated product solution was dried in vacuo to afford 4-[2-(1,2,3,5,6,7-hexahydropyrrolizin-8-ylmethoxy)-8-methyl-4-(1,4-oxazepan-4-yl)quinazolin-7-yl]quinazoline-6-carbonitrile (15 mg, 10%) as a yellow crystalline solid.

UPLC-MS (ES⁺, Method 1): 2.68 min, m/z 536.3 [M+H]⁺

¹H NMR (400 MHz, DMSO-d₆) δ/ppm 9.59 (s, 1H), 8.37 (dd, J=1.7, 8.8 Hz, 1H), 8.31 (d, J=8.7 Hz, 1H), 8.22-8.20 (m, 1H), 8.07-8.02 (m, 1H), 7.28-7.23 (m, 1H), 4.27 (s, 1H), 4.09 (s, 2H), 3.94-3.89 (m, 6H), 3.77-3.74 (m, 2H), 2.98-2.92 (m, 2H), 2.27-2.23 (m, 3H), 2.13-2.07 (m, 3H), 1.97-1.74 (m, 6H), 1.63-1.55 (m, 2H).

### Example 13

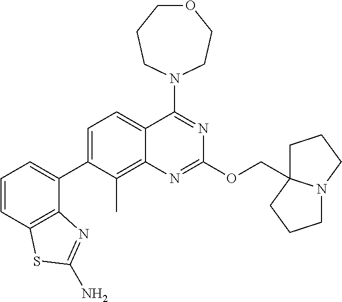 [SMILES: Cc1c(-c2cccc3sc(N)nc23)ccc2c(N3CCCOCC3)nc(OCC34CCCN3CCC4)nc12]

### 4-[2-(1,2,3,5,6,7-hexahydropyrrolizin-8-ylmethoxy)-8-methyl-4-(1,4-oxazepan-4yl)quinazolin-7-yl]-1,3-benzothiazol-2-amine

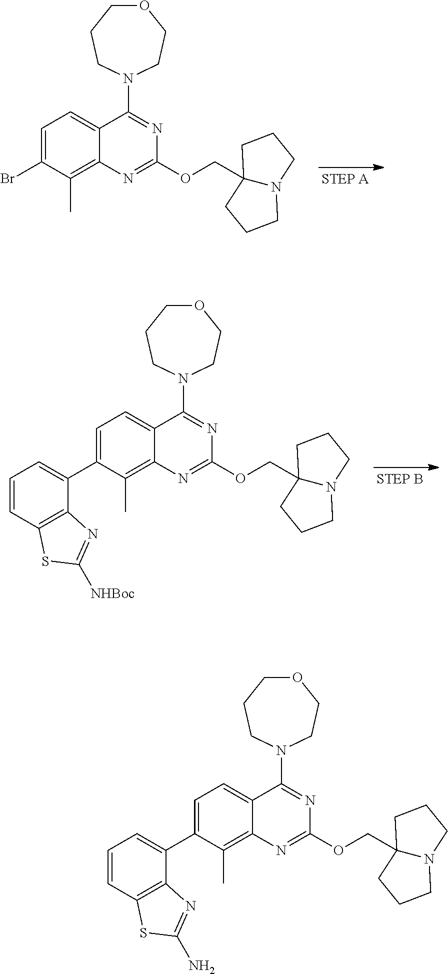 [SMILES: Cc1c(Br)ccc2c(N3CCCOCC3)nc(OCC34CCCN3CCC4)nc12 ; Cc1c(-c2cccc3sc(NC(=O)OC(C)(C)C)nc23)ccc2c(N3CCCOCC3)nc(OCC34CCCN3CCC4)nc12 ; Cc1c(-c2cccc3sc(N)nc23)ccc2c(N3CCCOCC3)nc(OCC34CCCN3CCC4)nc12]

STEP A, tert-butyl N-[4-[2-(1,2,3,5,6,7-hexahydropyrrolizin-8-ylmethoxy)-8-methyl-4-(1,4-oxazepan-4-yl)quinazolin-7-yl]-1,3-benzothiazol-2-yl]carbamate. To a degassed suspension of 4-[7-bromo-2-(1,2,3,5,6,7-hexahydropyrrolizin-8-ylmethoxy)-8-methyl-quinazolin-4-yl]-1,4-oxazepane (50 mg, 0.11 mmol), tert-butyl N-[4-(4,4,5,5-tetramethyl-1,3,2-dioxaborolan-2-yl)-1,3-benzothiazol-2-yl]carbamate (61.16 mg, 0.16 mmol) and cesium carbonate (105.9 mg, 0.33 mmol) in 1,4-dioxane (2 mL) and Water (0.2 mL) was added [1,1′-Bis(di-tert-butylphosphino)ferrocene]dichloropalladium(II) (7.06 mg, 0.01 mmol). The mixture was stirred at 95° C. overnight. The reaction mixture was diluted with EtOAc, filtered and concentrated under vacuum to afford a black oil. The crude was purified via flash column chromatography (5 g KP NH modified column) eluting from 10% to 100% EtOAc in pet ether followed by flushing through with 20% MeOH in DCM. The fractions containing product were combined and concentrated under vacuo to afford tert-butyl N-[4-[2-(1,2,3,5,6,7-hexahydropyrrolizin-8-ylmethoxy)-8-methyl-4-(1,4-oxazepan-4-yl)quinazolin-7-yl]-1,3-benzothiazol-2-yl]carbamate as a yellow oil (10 mg, 15%).

UPLC-MS (ES⁺, Method 2): 1.9 min, m/z 631.3 [M+H]⁺

STEP B, 4-[2-(1,2,3,5,6,7-hexahydropyrrolizin-8-ylmethoxy)-8-methyl-4-(1,4-oxazepan-4yl)quinazolin-7-yl]-1,3-benzothiazol-2-amine. To a solution of tert-butyl N-[4-[2-(1,2,3,5,6,7-hexahydropyrrolizin-8-ylmethoxy)-8-methyl-4-(1,4-oxazepan-4-yl)quinazolin-7-yl]-1,3-benzothiazol-2-yl]carbamate (10 mg, 0.02 mmol) in DCM (1 mL), trifluoroacetic acid (0.01 mL, 0.16 mmol) was added at room temperature and left to stir for 30 mins. The crude was concentrated and purified by flash column chromatography eluting 100% DCM to 20% MeOH in DCM. The desired fractions were concentrated to dryness, this afforded 4-[2-(1,2,3,5,6,7-hexahydropyrrolizin-8-ylmethoxy)-8-methyl-4-(1,4-oxazepan-4-yl)quinazolin-7-yl]-1,3-benzothiazol-2-amine (7 mg, 80%) as a white solid.

UPLC-MS (ES⁺, Method 1): 2.7 min, m/z 531.3 [M+H]⁺

¹H NMR (400 MHz, DMSO-d₆) δ/ppm 7.84 (d, J=8.8 Hz, 1H), 7.72 (dd, J=2.4, 6.7 Hz, 1H), 7.51 (s, 2H), 7.13-7.10 (m, 3H), 4.06-3.99 (m, 6H), 3.91-3.87 (m, 2H), 3.76-3.71 (m, 2H), 2.97-2.91 (m, 1H), 2.53 (d, J=1.8 Hz, 1H), 2.46-2.44 (m, 1H), 2.34-2.27 (m, 3H), 2.10-2.05 (m, 3H), 1.95-1.72 (m, 6H), 1.61-1.53 (m, 2H).

### Example 14

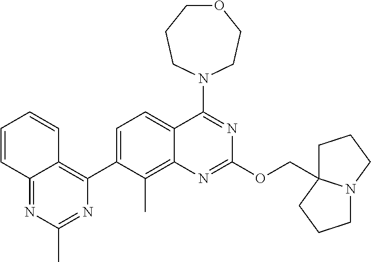 [SMILES: Cc1nc(-c2ccc3c(N4CCCOCC4)nc(OCC45CCCN4CCC5)nc3c2C)c2ccccc2n1]

### 4-[2-(1,2,3,5,6,7-hexahydropyrrolizin-8-ylmethoxy)-8-methyl-7-(2-methylquinazolin-4-yl)quinazolin-4-yl]-1,4-oxazepane

4-[2-(1,2,3,5,6,7-hexahydropyrrolizin-8-ylmethoxy)-8-methyl-7-(2-methylquinazolin-4-yl)quinazolin-4-yl]-1,4-oxazepane was prepared via analogy with Example 12, replacing 4-chloro-6-quinazolinecarbonitrile with 4-chloro-2-methylquinazoline.

UPLC-MS (ES⁺, Method 1): 2.6 min, m/z 525.3 [M+H]⁺

¹H NMR (400 MHz, DMSO-d₆) δ/ppm 8.05-7.98 (m, 3H), 7.63-7.53 (m, 2H), 7.21 (d, J=8.6 Hz, 1H), 4.10-4.03 (m, 6H), 3.92 (dd, J=4.5, 4.5 Hz, 2H), 3.75 (dd, J=5.3, 5.3 Hz, 2H), 2.97-2.91 (m, 2H), 2.84 (s, 3H), 2.55-2.52 (m, 2H), 2.23, 2.21 (s, 3H), 2.12-2.06 (m, 2H), 1.96-1.72 (m, 6H), 1.62-1.54 (m, 2H).

### Example 15

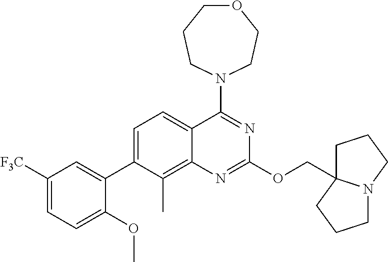 [SMILES: COc1ccc(C(F)(F)F)cc1-c1ccc2c(N3CCCOCC3)nc(OCC34CCCN3CCC4)nc2c1C]

### 4-[2-(1,2,3,5,6,7-hexahydropyrrolizin-8-ylmethoxy)-7-[2-methoxy-5-(trifluoromethyl)phenyl]-8-methyl-quinazolin-4-yl]-1,4-oxazepane

4-[2-(1,2,3,5,6,7-hexahydropyrrolizin-8-ylmethoxy)-7-[2-methoxy-5-(trifluoromethyl)phenyl]-8-methyl-quinazolin-4-yl]-1,4-oxazepane was prepared via analogy with Example 13 STEP A, replacing tert-butyl N-[4-(4,4,5,5-tetramethyl-1,3,2-dioxaborolan-2-yl)-1,3-benzothiazol-2-yl]carbamate with 2-methoxy-5-(trifluoromethyl)phenylboronic acid.

UPLC-MS (ES⁺, Method 1): 3.2 min, m/z 557.4 [M+H]⁺

¹H NMR (400 MHz, DMSO-d₆) δ/ppm 7.87 (d, J=8.7 Hz, 1H), 7.80 (dd, J=1.8, 8.7 Hz, 1H), 7.47 (d, J=2.2 Hz, 1H), 7.35 (d, J=8.7 Hz, 1H), 7.05 (d, J=8.6 Hz, 1H), 4.07-3.98 (m, 6H), 3.82 (m, 5H), 3.73 (dd, J=4.8, 5.8 Hz, 2H), 2.98-2.90 (m, 2H), 2.58-2.52 (m, 2H), 2.25 (s, 3H), 2.11-2.03 (m, 2H), 1.95-1.71 (m, 6H), 1.61-1.52 (m, 2H).

### Example 16

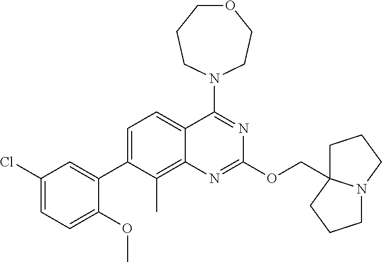 [SMILES: COc1ccc(Cl)cc1-c1ccc2c(N3CCCOCC3)nc(OCC34CCCN3CCC4)nc2c1C]

### 4-[7-(5-chloro-2-methoxy-phenyl)-2-(1,2,3,5,6,7-hexahydropyrrolizin-8-ylmethoxy)-8-methyl-quinazolin-4-yl]-1,4-oxazepane

4-[7-(5-chloro-2-methoxy-phenyl)-2-(1,2,3,5,6,7-hexahydropyrrolizin-8-ylmethoxy)-8-methyl-quinazolin-4-yl]-1,4-oxazepane was prepared via analogy with Example 13 STEP A, replacing tert-butyl N-[4-(4,4,5,5-tetramethyl-1,3,2-dioxaborolan-2-yl)-1,3-benzothiazol-2-yl]carbamate with 5-chloro-2-methoxyphenylboronic acid.

UPLC-MS (ES⁺, Method 1): 3.2 min, m/z 523.3 [M+H]⁺

¹H NMR (400 MHz, DMSO-d₆) δ/ppm 7.84 (d, J=8.7 Hz, 1H), 7.46 (d, J=2.2 Hz, 1H), 7.20-7.15 (m, 2H), 7.05 (d, J=8.6 Hz, 1H), 4.07-3.98 (m, 6H), 3.82 (m, 5H), 3.73 (dd, J=4.8, 5.8 Hz, 2H), 2.98-2.90 (m, 2H), 2.58-2.52 (m, 2H), 2.25 (s, 3H), 2.11-2.03 (m, 2H), 1.95-1.71 (m, 6H), 1.61-1.52 (m, 2H).

### Example 17

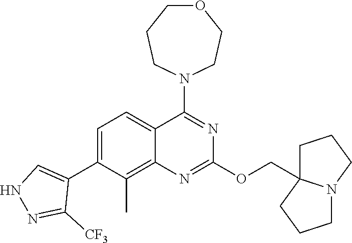 [SMILES: Cc1c(-c2c[nH]nc2C(F)(F)F)ccc2c(N3CCCOCC3)nc(OCC34CCCN3CCC4)nc12]

### 4-[2-(1,2,3,5,6,7-hexahydropyrrolizin-8-ylmethoxy)-8-methyl-7-[3-(trifluoromethyl)-1H-pyrazol-4-yl]quinazolin-4-yl]-1,4-oxazepane

4-[2-(1,2,3,5,6,7-hexahydropyrrolizin-8-ylmethoxy)-8-methyl-7-[3-(trifluoromethyl)-1H-pyrazol-4-yl]quinazolin-4-yl]-1,4-oxazepane was prepared via analogy with Example 13 STEP A, replacing tert-butyl N-[4-(4,4,5,5-tetramethyl-1,3,2-dioxaborolan-2-yl)-1,3-benzothiazol-2-yl]carbamate with 3-trifluoromethyl-1H-pyrazole-4-boronic acid pinacol ester.

UPLC-MS (ES⁺, Method 1): 2.6 min, m/z 517.2 [M+H]⁺

¹H NMR (400 MHz, DMSO-d₆) δ/ppm 13.87-13.83 (bs, 1H), 8.08 (d, J=0.6 Hz, 1H), 7.85 (d, J=8.7 Hz, 1H), 7.06 (d, J=8.7 Hz, 1H), 4.05-3.73 (m, 10H), 2.97-2.90 (m, 2H), 2.53 (m, 2H), 2.47 (d, J=1.8 Hz, 3H), 2.35 (s, 1H), 2.34 (s, 3H), 2.09-1.52 (m, 10H).

### Example 18

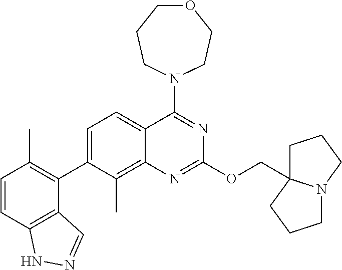 [SMILES: Cc1ccc2[nH]ncc2c1-c1ccc2c(N3CCCOCC3)nc(OCC34CCCN3CCC4)nc2c1C]

### 4-[2-(1,2,3,5,6,7-hexahydropyrrolizin-8-ylmethoxy)-8-methyl-7-(5-methyl-1H-indazol-4-yl)quinazolin-4-yl]-1,4-oxazepane

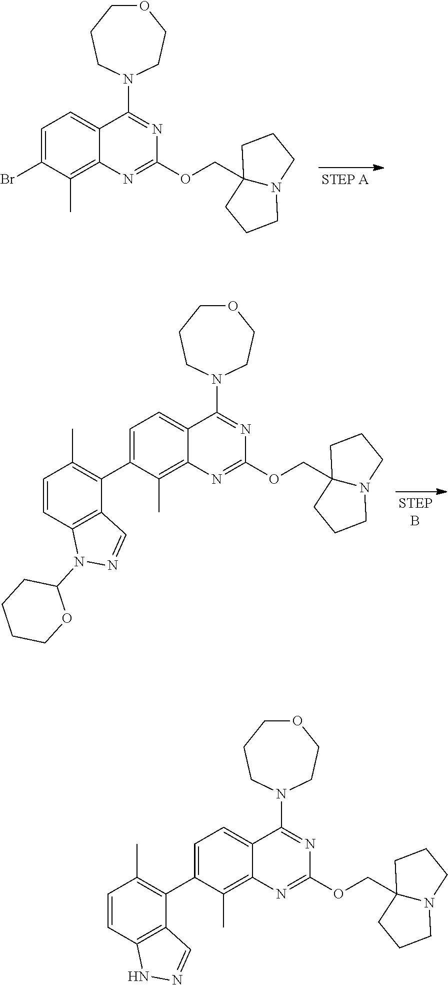 [SMILES: Cc1c(Br)ccc2c(N3CCCOCC3)nc(OCC34CCCN3CCC4)nc12 ; Cc1ccc2c(cnn2C2CCCCO2)c1-c1ccc2c(N3CCCOCC3)nc(OCC34CCCN3CCC4)nc2c1C ; Cc1ccc2[nH]ncc2c1-c1ccc2c(N3CCCOCC3)nc(OCC34CCCN3CCC4)nc2c1C]

STEP A, 4-[2-(1,2,3,5,6,7-hexahydropyrrolizin-8-ylmethoxy)-8-methyl-7-(5-methyl-1-tetrahydropyran-2-yl-indazol-4-yl)quinazolin-4-yl]-1,4-oxazepane. A suspension of 5-methyl-1-tetrahydropyran-2-yl-4-(4,4,5,5-tetramethyl-1,3,2-dioxaborolan-2-yl)indazole (215.2 mg, 0.63 mmol), 4-[7-bromo-2-(1,2,3,5,6,7-hexahydropyrrolizin-8-ylmethoxy)-8-methyl-quinazolin-4-yl]-1,4-oxazepane (241.7 mg, 0.52 mmol) and cesium carbonate (512.1 mg, 1.57 mmol) in a mixture of 1,4-dioxane (4 mL) and water (1.38 mL) was de-gassed with nitrogen for 15 minutes. [1,1′-Bis(diphenylphosphino)ferrocene]palladium(II) chloride dichloromethane complex (85.58 mg, 0.1 mmol) was added and the reaction was allowed to stir at 95° C. overnight. The reaction was cooled to room temperature and filtered through and plug of celite. The filtrate was concentrated to dryness and the resultant residue was re-dissolved in DCM for dry loading on silica. The product was purified by flash column chromatography on silica gel (40 g) eluting with 0-60% (20% 1M ammonia in MeOH) in DCM. The desired fractions were combined and concentrated under reduced pressure to afford 4-[2-(1,2,3,5,6,7-hexahydropyrrolizin-8-ylmethoxy)-8-methyl-7-(5-methyl-1-tetrahydropyran-2-yl-indazol-4-yl)quinazolin-4-yl]-1,4-oxazepane (37.3 mg, 12% yield) as a brown flaky solid.

UPLC-MS (ES⁺, Method 2): 1.6 min, m/z 597.3 [M+H]⁺

¹H NMR (400 MHz, DMSO-d₆) δ/ppm 8.00-7.93 (m, 1H), 7.69 (d, J=8.7 Hz, 1H), 7.43-7.40 (m, 2H), 7.09-7.02 (m, 1H), 5.89-5.83 (m, 1H), 4.10-4.00 (m, 5H), 3.94-3.86 (m, 3H), 3.78-3.72 (m, 3H), 2.53-2.46 (m, 3H), 2.44-2.36 (m, 2H), 2.17 (d, J=8.6 Hz, 3H), 2.11 (s, 3H) 2.09-1.93 (m, 7H), 1.93-1.68 (m, 6H), 1.63-1.54 (m, 3H).

STEP B, 4-[2-(1,2,3,5,6,7-hexahydropyrrolizin-8-ylmethoxy)-8-methyl-7-(5-methyl-1H-indazol-4-yl)quinazolin-4-yl]-1,4-oxazepane. 4-[2-(1,2,3,5,6,7-hexahydropyrrolizin-8-ylmethoxy)-8-methyl-7-(5-methyl-1-tetrahydropyran-2-yl-indazol-4-yl)quinazolin-4-yl]-1,4-oxazepane (36.3 mg, 0.06 mmol) was dissolved in a mixture of DCM (0.6 mL) and methanol (0.01 mL) and the reaction was cooled to 0° C. Triethylsilane (0.1 mL, 0.61 mmol) was added, followed by slow addition of trifluoroacetic acid (0.47 mL, 6.08 mmol). The reaction was stirred at 0° C. for 5 minutes and the reaction was then warmed to room temperature. The reaction was allowed to stir at 25° C. for 2.5 hrs. Celite was added to the reaction and it was concentrated to dryness. The product was purified by reverse phase chromatography eluting with 2-50% MeCN (0.1% formic acid) in Water (0.1% formic acid) with fractions containing product isolated by SCX (methanol wash (x2) followed by 1 M NH₃ in MeOH (x2)). The filtrate was concentrated in vacuo to afford 4-[2-(1,2,3,5,6,7-hexahydropyrrolizin-8-ylmethoxy)-8-methyl-7-(5-methyl-1H-indazol-4-yl)quinazolin-4-yl]-1,4-oxazepane (29.5 mg, 94.6%) as an off-white powder.

UPLC-MS (ES⁺, Method 1): 2.6 min, m/z 513.4 [M+H]⁺

¹H NMR (400 MHz, DMSO-d₆) δ/ppm 13.08 (s, 1H), 7.94 (d, J=8.7 Hz, 1H), 7.49 (d, J=8.6 Hz, 1H), 7.39 (s, 1H), 7.34 (d, J=8.6 Hz, 1H), 7.05 (d, J=8.6 Hz, 1H), 4.07-4.01 (m, 6H), 3.92-3.88 (m, 2H), 3.77-3.72 (m, 2H), 2.97-2.89 (m, 2H), 2.57-2.46 (m, 2H), 2.16 (s, 3H), 2.12-2.06 (m, 5H), 1.96-1.88 (m, 2H), 1.85-1.71 (m, 4H), 1.61-1.52 (m, 2H).

### Example 19

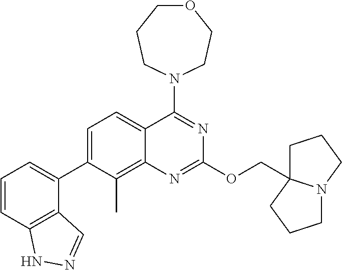 [SMILES: Cc1c(-c2cccc3[nH]ncc23)ccc2c(N3CCCOCC3)nc(OCC34CCCN3CCC4)nc12]

### 4-[2-(1,2,3,5,6,7-hexahydropyrrolizin-8-ylmethoxy)-7-(1H-indazol-4-yl)-8-methyl-quinazolin-4-yl]-1,4-oxazepane

4-[2-(1,2,3,5,6,7-hexahydropyrrolizin-8-ylmethoxy)-7-(1H-indazol-4-yl)-8-methyl-quinazolin-4-yl]-1,4-oxazepane was prepared by analogy with example 18, replacing 5-methyl-1-tetrahydropyran-2-yl-4-(4,4,5,5-tetramethyl-1,3,2-dioxaborolan-2-yl)indazole in STEP A with 1-tetrahydropyran-2-yl-4-(4,4,5,5-tetramethyl-1,3,2-dioxaborolan-2-yl)indazole.

UPLC-MS (ES⁺, Method 1): 2.7 min, m/z 499.3 [M+H]⁺

¹H NMR (400 MHz, DMSO-d₆) δ/ppm 13.25 (s, 1H), 7.94 (d, J=8.7 Hz, 1H), 7.72 (s, 1H), 7.60 (d, J=8.4 Hz, 1H), 7.47 (dd, J=7.0, 8.3 Hz, 1H), 7.22 (d, J=8.7 Hz, 1H), 7.07 (dd, J=0.7, 7.0 Hz, 1H), 4.09-4.01 (m, 6H), 3.92-3.88 (m, 2H), 3.75 (dd, J=4.8, 5.8 Hz, 2H), 2.99-2.92 (m, 2H), 2.60-2.52 (m, 3H), 2.36 (s, 3H), 2.12-2.05 (m, 2H), 1.98-1.73 (m, 6H), 1.63-1.55 (m, 2H).

### Example 20

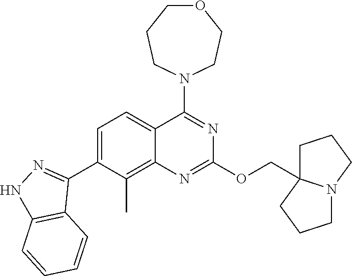 [SMILES: Cc1c(-c2n[nH]c3ccccc23)ccc2c(N3CCCOCC3)nc(OCC34CCCN3CCC4)nc12]

### 4-[2-(1,2,3,5,6,7-hexahydropyrrolizin-8-ylmethoxy)-7-(1H-indazol-3-yl)-8-methyl-quinazolin-4-yl]-1,4-oxazepane

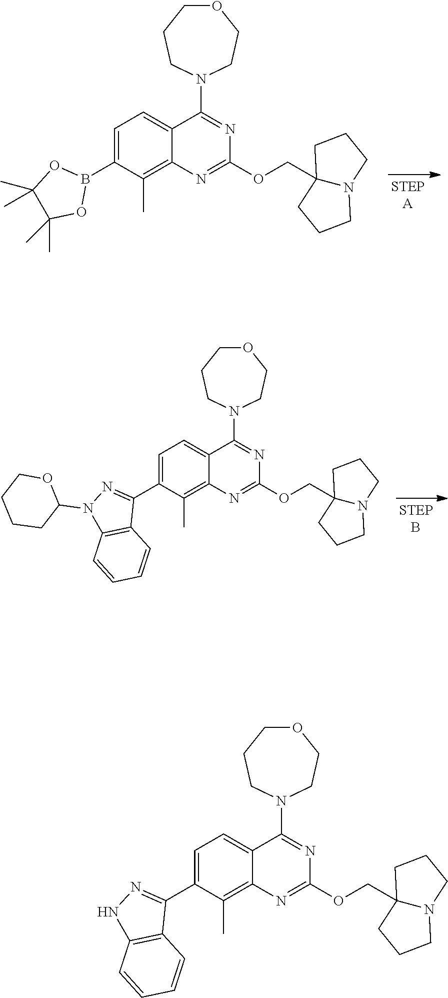 [SMILES: Cc1c(B2OC(C)(C)C(C)(C)O2)ccc2c(N3CCCOCC3)nc(OCC34CCCN3CCC4)nc12 ; Cc1c(-c2nn(C3CCCCO3)c3ccccc23)ccc2c(N3CCCOCC3)nc(OCC34CCCN3CCC4)nc12 ; Cc1c(-c2n[nH]c3ccccc23)ccc2c(N3CCCOCC3)nc(OCC34CCCN3CCC4)nc12]

STEP A, 4-[2-(1,2,3,5,6,7-hexahydropyrrolizin-8-ylmethoxy)-8-methyl-7-(1-tetrahydropyran-2-ylindazol-3-yl)quinazolin-4-yl]-1,4-oxazepane. To a degassed suspension of 4-[2-(1,2,3,5,6,7-hexahydropyrrolizin-8-ylmethoxy)-8-methyl-7-(4,4,5,5-tetramethyl-1,3,2-dioxaborolan-2-yl)quinazolin-4-yl]-1,4-oxazepane (150 mg, 0.3 mmol), 3-bromo-1-tetrahydropyran-2-yl-indazole (124.41 mg, 0.44 mmol) and cesium carbonate (288.4 mg, 0.89 mmol) in 1,4-dioxane (5 mL) and water (0.5 mL) was added [1,1′-Bis(di-tert-butylphosphino)ferrocene]dichloropalladium(II) (19.23 mg, 0.03 mmol). The mixture was stirred at 95° C. overnight. The reaction mixture was diluted with EtOAc, filtered and concentrated in vacuo. The crude material was purified via flash column chromatography (5 g KP NH modified column) eluting from 10% to 100% EtOAc in pet ether followed by 20% MeOH in DCM. The fractions containing product were combined and concentrated to afford 4-[2-(1,2,3,5,6,7-hexahydropyrrolizin-8-ylmethoxy)-8-methyl-7-(1-tetrahydropyran-2-ylindazol-3-yl)quinazolin-4-yl]-1,4-oxazepane (81 mg, 47%) as a brown oil.

UPLC-MS (ES⁺, Method 2): 1.7 min, m/z 583.3 [M+H]⁺

STEP B, 4-[2-(1,2,3,5,6,7-hexahydropyrrolizin-8-ylmethoxy)-7-(1H-indazol-3-yl)-8-methyl-quinazolin-4-yl]-1,4-oxazepane. To a solution of 4-[2-(1,2,3,5,6,7-hexahydropyrrolizin-8-ylmethoxy)-8-methyl-7-(1-tetrahydropyran-2-ylindazol-3-yl)quinazolin-4-yl]-1,4-oxazepane (80 mg, 0.14 mmol) in a mixture of DCM (2 mL) methanol (2 mL), trifluoroacetic acid (0.11 mL, 1.37 mmol) was added at room temperature and left to stir overnight. The reaction was heated up to 40° C. and 4N HCl in dioxane (0.34 mL, 1.37 mmol) was added to the reaction. This was left to stir at room temperature over the weekend. The crude was purified by flash column chromatography eluting 10%-100% EtOAc in Pet ether, followed by 20% MeOH in DCM. The desired fractions were concentrated to afford 4-[2-(1,2,3,5,6,7-hexahydropyrrolizin-8-ylmethoxy)-7-(1H-indazol-3-yl)-8-methyl-quinazolin-4-yl]-1,4-oxazepane (4 mg. 6%) as a white solid.

UPLC-MS (ES⁺, Method 1): 2.7 min, m/z 499.3 [M+H]⁺

¹H NMR (400 MHz, DMSO-d₆) δ/ppm 3.35 (s, 1H), 7.96 (d, J=8.7 Hz, 1H), 7.65-7.60 (m, 2H), 7.45-7.38 (m, 2H), 7.20-7.16 (m, 1H), 4.09-4.01 (m, 6H), 3.92-3.88 (m, 2H), 3.75 (dd, J=4.8, 5.8 Hz, 2H), 2.99-2.92 (m, 2H), 2.60-2.52 (m, 3H), 2.36 (s, 3H), 2.12-2.05 (m, 2H), 1.98-1.73 (m, 6H), 1.63-1.55 (m, 2H).

### Example 21

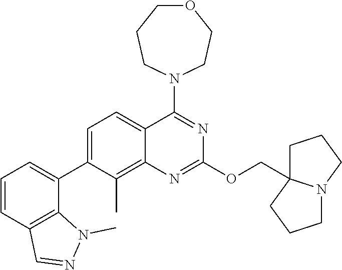 [SMILES: Cc1c(-c2cccc3cnn(C)c23)ccc2c(N3CCCOCC3)nc(OCC34CCCN3CCC4)nc12]

### 4-[2-(1,2,3,5,6,7-hexahydropyrrolizin-8-ylmethoxy)-8-methyl-7-(1-methylindazol-7-yl)quinazolin-4-yl]-1,4-oxazepane

4-[2-(1,2,3,5,6,7-hexahydropyrrolizin-8-ylmethoxy)-8-methyl-7-(1-methylindazol-7-yl)quinazolin-4-yl]-1,4-oxazepane was prepared by analogy with example 12, replacing 4-chloro-6-quinazolinecarbonitrile with 7-Bromo-1-methyl-1H-indazole.

UPLC-MS (ES⁺, Method 1): 2.9 min, m/z 513.4 [M+H]⁺

¹H NMR (400 MHz, DMSO-d₆) δ/ppm 8.15 (s, 1H), 7.95 (d, J=8.7 Hz, 1H), 7.84 (dd, J=1.1, 7.9 Hz, 1H), 7.26-7.16 (m, 3H), 4.07-4.02 (m, 6H), 3.93-3.88 (m, 2H), 3.78-3.73 (m, 2H), 3.43 (s, 3H), 2.97-2.90 (m, 2H), 2.56-2.52 (m, 2H), 2.22 (s, 3H), 2.12-2.05 (m, 2H), 1.96-1.71 (m, 6H), 1.61-1.53 (m, 2H).

### Example 22

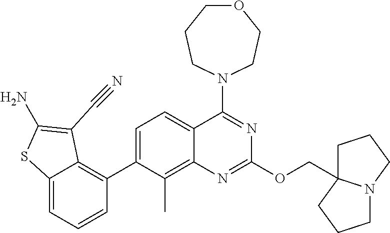 [SMILES: Cc1c(-c2cccc3sc(N)c(C#N)c23)ccc2c(N3CCCOCC3)nc(OCC34CCCN3CCC4)nc12]

### 2-amino-4-[2-(1,2,3,5,6,7-hexahydropyrrolizin-8-ylmethoxy)-8-methyl-4-(1,4-oxazepan-4-yl)quinazolin-7-yl]benzothiophene-3-carbonitrile

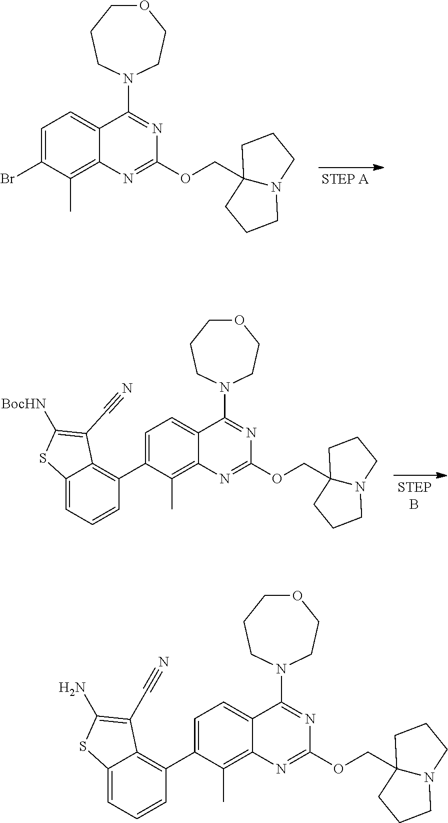 [SMILES: Cc1c(Br)ccc2c(N3CCCOCC3)nc(OCC34CCCN3CCC4)nc12 ; Cc1c(-c2cccc3sc(NC(=O)OC(C)(C)C)c(C#N)c23)ccc2c(N3CCCOCC3)nc(OCC34CCCN3CCC4)nc12 ; Cc1c(-c2cccc3sc(N)c(C#N)c23)ccc2c(N3CCCOCC3)nc(OCC34CCCN3CCC4)nc12]

STEP A, tert-butyl N-[3-cyano-4-[2-(1,2,3,5,6,7-hexahydropyrrolizin-8-ylmethoxy)-8-methyl-4-(1,4-oxazepan-4-yl)quinazolin-7-yl]benzothiophene-2-yl]carbamate. To a degassed suspension of 4-[7-bromo-2-(1,2,3,5,6,7-hexahydropyrrolizin-8-ylmethoxy)-8-methyl-quinazolin-4-yl]-1,4-oxazepane (50 mg, 0.11 mmol), tert-butyl N-[3-cyano-4-(4,4,5,5-tetramethyl-1,3,2-dioxaborolan-2-yl)benzothiophen-2-yl]carbamate (173.52 mg, 0.43 mmol) and cesium carbonate (105.9 mg, 0.33 mmol) in 1,4-dioxane (2.5 mL) and water (0.25 mL) was added [1,1′-Bis(diphenylphosphino)ferrocene]dichloropalladium II (15.9 mg, 0.02 mmol). The mixture was stirred at 95° C. overnight. The reaction was diluted with 20% MeOH in DCM, filtered and concentrated to afford a black solid. The crude was purified by flash column chromatography (5 g KP NH modified column) eluting 0-100% EtOAc in pet ether followed by 20% MeOH in DCM. The desired fractions were combined and concentrated under vacuum to afford tert-butyl N-[3-cyano-4-[2-(1,2,3,5,6,7-hexahydropyrrolizin-8-ylmethoxy)-8-methyl-4-(1,4-oxazepan-4-yl)quinazolin-7-yl]benzothiophen-2-yl]carbamate (30 mg, 43%) as a yellow oil.

UPLC-MS (ES⁺, Method 2): 1.9 min, m/z 655.5 [M+H]⁺

STEP B, 2-amino-4-[2-(1,2,3,5,6,7-hexahydropyrrolizin-8-ylmethoxy)-8-methyl-4-(1,4-oxazepan-4-yl)quinazolin-7-yl]benzothiophene-3-carbonitrile. To a solution of tert-butyl N-[3-cyano-4-[2-(1,2,3,5,6,7-hexahydropyrrolizin-8-ylmethoxy)-8-methyl-4-(1,4-oxazepan-4-yl)quinazolin-7-yl]benzothiophen-2-yl]carbamate (30 mg, 0.05 mmol) in DCM (1 mL), trifluoroacetic acid (0.04 mL, 0.46 mmol) was added at room temperature and left to stir for 30 mins. The mixture was concentrated and the crude product was purified by flash column chromatography eluting 10%-100% EtOAc in pet ether to followed by 20% MeOH in DCM. The desired fractions were combined and concentrated to dryness to afford an off-white residue. This was redissolved in DCM (1 ml) and passed through an SXC column (NH₃ in MeOH) before removing solvent from the product containing fraction. This afforded 2-amino-4-[2-(1,2,3,5,6,7-hexahydropyrrolizin-8-ylmethoxy)-8-methyl-4-(1,4-oxazepan-4-yl)quinazolin-7-yl]benzothiophene-3-carbonitrile (4 mg, 15%) as an off-white solid.

UPLC-MS (ES⁺, Method 1): 2.9 min, m/z 555.3 [M+H]⁺

¹H NMR (400 MHz, DMSO-d₆) δ/ppm 7.85 (d, J=8.4 Hz, 1H), 7.72 (dd, J=1.1, 7.9 Hz, 1H), 7.67 (s, 2H), 7.20 (dd, J=7.6, 7.6 Hz, 1H), 7.09-7.04 (m, 2H), 4.09-3.99 (m, 6H), 3.92-3.88 (m, 2H), 3.77-3.72 (m, 2H), 3.05-2.94 (m, 2H), 2.55-2.50 (m, 2H), 2.23 (s, 3H), 2.07-2.06 (m, 2H), 1.91 (s, 2H), 1.84-1.78 (m, 4H), 1.71-1.60 (m, 2H).

### Example 23

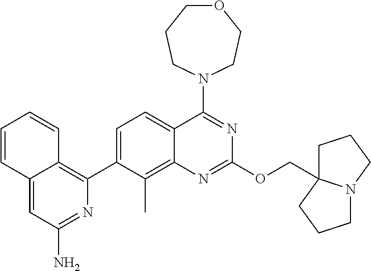 [SMILES: Cc1c(-c2nc(N)cc3ccccc23)ccc2c(N3CCCOCC3)nc(OCC34CCCN3CCC4)nc12]

### 1-[2-(1,2,3,5,6,7-hexahydropyrrolizin-8-ylmethoxy)-8-methyl-4-(1,4-oxazepan-4-yl)quinazolin-7-yl]isoquinolin-3-amine

1-[2-(1,2,3,5,6,7-hexahydropyrrolizin-8-ylmethoxy)-8-methyl-4-(1,4-oxazepan-4-yl)quinazolin-7-yl]isoquinolin-3-amine was prepared via analogy with Example 12, replacing 4-chloro-6-quinazolinecarbonitrile with 3-Amino-1-bromoisoquinoline.

UPLC-MS (ES⁺, Method 1): 2.6 min, m/z 525.3 [M+H]⁺

¹H NMR (400 MHz, DMSO-d₆) δ/ppm 7.96-7.93 (m, 1H), 7.62-7.60 (m, 1H), 7.46-7.40 (m, 1H), 7.20-7.03 (m, 3H), 6.69 (s, 1H), 6.09-6.01 (bs, 2H), 4.10-4.03 (m, 6H), 3.92 (dd, J=4.5, 4.5 Hz, 2H), 3.75 (dd, J=5.3, 5.3 Hz, 2H), 2.97-2.91 (m, 2H), 2.55-2.52 (m, 2H), 2.23, 2.21 (s, 3H), 2.12-2.06 (m, 2H), 1.96-1.72 (m, 6H), 1.62-1.54 (m, 2H).

### Example 24

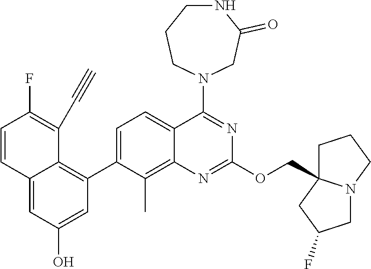 [SMILES: C#Cc1c(F)ccc2cc(O)cc(-c3ccc4c(N5CCCNC(=O)C5)nc(OC[C@@]56CCCN5C[C@H](F)C6)nc4c3C)c12]

### 4-[7-(8-ethynyl-7-fluoro-3-hydroxy-1-naphthyl)-2-[[rac-(2R,8S)-2-fluoro-1,2,3,5,6,7-hexahydropyrrolizin-8-yl]methoxy]-8-methyl-quinazolin-4-yl]-1,4-diazepan-2-one

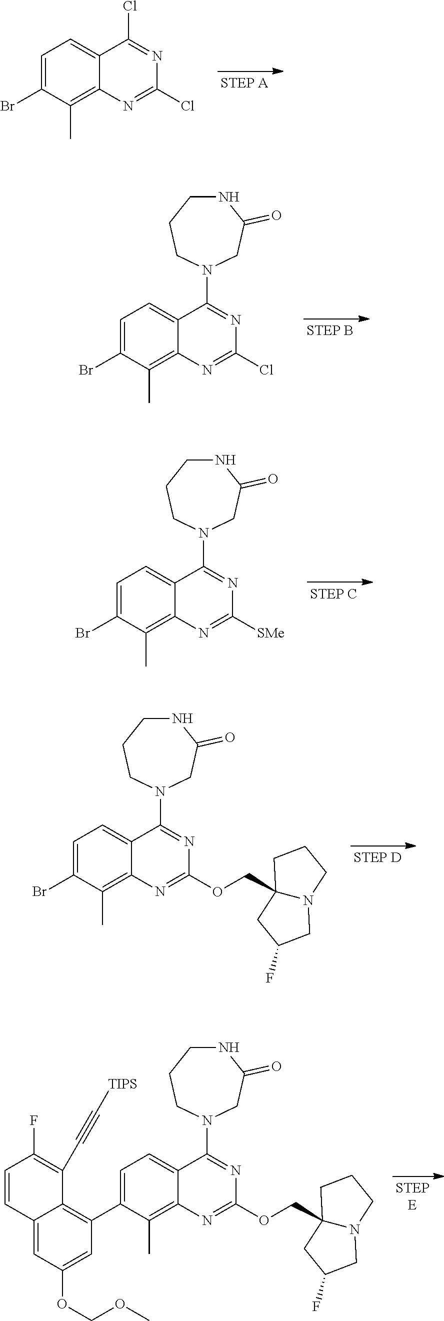

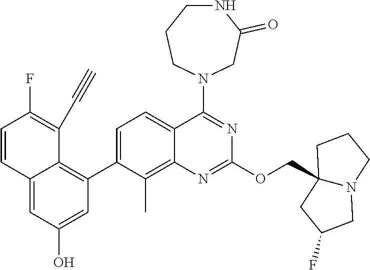 [SMILES: C#Cc1c(F)ccc2cc(O)cc(-c3ccc4c(N5CCCNC(=O)C5)nc(OC[C@@]56CCCN5C[C@H](F)C6)nc4c3C)c12]

STEP A, 4-(7-bromo-2-chloro-8-methyl-quinazolin-4-yl)-1,4-diazepan-2-one. 1,4-Diazepan-2-one (234.59 mg, 2.06 mmol) was added to a stirred solution of 7-bromo-2,4-dichloro-8-methylquinazoline (300 mg, 1.03 mmol) and N,N-diisopropylethylamine (0.6 mL, 3.43 mmol) in DCM (7 mL) at 0° C. and the reaction was left to stir for 2 h. The reaction was directly dry loaded onto silica. The product was purified by flash column chromatography (12 g SiO₂) eluting first with 20-100% EtOAc in petroleum ether, followed by 0-20% MeOH in DCM. The desired fractions were combined and concentrated under reduced pressure to afford 4-(7-bromo-2-chloro-8-methyl-quinazolin-4-yl)-1,4-diazepan-2-one (382.5 mg, 100% yield) as a powdery white solid.

UPLC-MS (ES⁺, Method 2): 1.7 min, m/z 371.0 [M+H]⁺

STEP B, 4-(7-bromo-8-methyl-2-methylsulfanyl-quinazolin-4-yl)-1,4-diazepan-2-one. Sodium methanethiolate (135.54 mg, 1.93 mmol) was added to a solution of 4-(7-bromo-2-chloro-8-methyl-quinazolin-4-yl)-1,4-diazepan-2-one (357.4 mg, 0.97 mmol) in dry DMF (10 mL) and the reaction was allowed to stir at room temperature for 1 h. The reaction was concentrated to dryness. The residue was taken up in EtOAc (30 mL) and silica was added for dry loading.

The product was purified by flash column chromatorgaphy on silica gel (12 g) eluting first with 20-100% EtOAc in petroleum ether followed by 0-20% MeOH in DCM. The desired fractions were combined and concentrated under reduced pressure to afford 4-(7-bromo-8-methyl-2-methylsulfanyl-quinazolin-4-yl)-1,4-diazepan-2-one (312.4 mg, 0.81 mmol, 84% yield) as a pale yellow powder.

UPLC-MS (ES⁺, Method 2): 1.8 min, m/z 382.4 [M+H]⁺

¹H NMR (400 MHz, DMSO-d₆) δ/ppm 7.87 (d, J=9.1 Hz, 1H), 7.77-7.71 (m, 1H), 7.53 (d, J=9.0 Hz, 1H), 4.29 (s, 2H), 3.99-3.92 (m, 2H), 3.09-3.01 (m, 2H), 2.64 (s, 3H) 2.53 (s, 3H), 2.08-1.98 (m, 2H).

STEP C, 4-[7-bromo-8-methyl-2-[[rac-(2R,8S)-2-fluoro-1,2,3,5,6,7-hexahydropyrrolizin-8-yl]methoxy]quinazolin-4-yl]-1,4-diazepan-2-one. m-chloroperbenzoic acid, m-CPBA (565.56 mg, 3.28 mmol) was added to a stirring solution of 4-(7-bromo-8-methyl-2-methylsulfanyl-quinazolin-4-yl)-1,4-diazepan-2-one (312.4 mg, 0.82 mmol) in DCM (25 mL) at 0° C. The reaction stirred for 2.5 hrs. The reaction was warmed to room temperature and allowed to stir overnight. The reaction was concentrated to dryness. The resultant residue was dissolved in dry THF (30 mL) and [rac-(2R,8S)-2-fluoro-1,2,3,5,6,7-hexahydropyrrolizin-8-yl]methanol (391.31 mg, 2.46 mmol) was added. Sodium tert-butoxide (1.23 mL, 2.46 mmol) (2M in THF) was added dropwise and the reaction was allowed to stir for 2.5 hrs. The reaction was concentrated to dryness. The resultant residue was re-dissolved in DCM (5 mL) and THF (5 mL). Silica was added for dry loading. The product was purified by flash column chromatography on silica gel (25 g) eluting with 0-20% (1M ammonia in MeOH) in DCM. The desired fractions were united and concentrated to dryness to afford 4-[7-bromo-8-methyl-2-[[rac-(2R,8S)-2-fluoro-1,2,3,5,6,7-hexahydropyrrolizin-8-yl]methoxy]quinazolin-4-yl]-1,4-diazepan-2-one (304.3 mg, 0.618 mmol, 75% yield) as a translucent yellow gum.

UPLC-MS (ES⁺, Method 2): 1.4 min, m/z 494.0 [M+H]⁺

STEP D, 4-[7-[7-fluoro-3-(methoxymethoxy)-8-(2-triisopropylsilylethynyl)-1-naphthyl]-8-methyl-2-[[rac-(2R,8S)-2-fluoro-1,2,3,5,6,7-hexahydropyrrolizin-8-yl]methoxy]quinazolin-4-yl]-1,4-diazepan-2-one. A suspension of 2-[2-fluoro-6-(methoxymethoxy)-8-(4,4,5,5-tetramethyl-1,3,2-dioxaborolan-2-yl)-1-naphthyl]ethynyl-triisopropyl-silane (237.65 mg, 0.46 mmol), 4-[7-bromo-8-methyl-2-[[rac-(2R,8S)-2-fluoro-1,2,3,5,6,7-hexahydropyrrolizin-8-yl]methoxy]quinazolin-4-yl]-1,4-diazepan-2-one (152.2 mg, 0.31 mmol) and cesium carbonate (201.43 mg, 0.62 mmol) in a mixture of 1,4-dioxane (5 mL) and Water (2 mL) was de-gassed with nitrogen for 15 minutes. [1,1′-Bis(di-tert-butylphosphino)ferrocene]dichloropalladium(II) (40.29 mg, 0.06 mmol) was added and the reaction was heated to 90° C. The reaction was allowed to stir overnight. The reaction was cooled to room temperature and was filtered through a plug of Celite (washed twice with MeOH). The filtrate was concentrated to dryness.

The resulting residue was taken up in a mixture of MeOH and DCM, and silica was added for dry loading. The product was purified by flash column chromatrography on silica gel (12 g) eluting with 0-20% (1M ammonia in MeOH) in DCM. The desired fractions were combined and concentrated under reduced pressure to afford 4-[7-[7-fluoro-3-(methoxymethoxy)-8-(2-triisopropylsilylethynyl)-1-naphthyl]-8-methyl-2-[[rac-(2R,8S)-2-fluoro-1,2,3,5,6,7-hexahydropyrrolizin-8-yl]methoxy]quinazolin-4-yl]-1,4-diazepan-2-one (32.7 mg, 13% yield) as a brown residue.

UPLC-MS (ES⁺, Method 2): 1.92 min, m/z 798.7 [M+H]⁺

STEP E, 4-[7-(8-ethynyl-7-fluoro-3-hydroxy-1-naphthyl)-2-[[rac-(2R,8S)-2-fluoro-1,2,3,5,6,7-hexahydropyrrolizin-8-yl]methoxy]-8-methyl-quinazolin-4-yl]-1,4-diazepan-2-one. A solution of 4-[2-[[rac-(2R,8S)-2-fluoro-1,2,3,5,6,7-hexahydropyrrolizin-8-yl]methoxy]-7-[7-fluoro-3-(methoxymethoxy)-8-(2-triisopropylsilylethynyl)-1-naphthyl]-8-methyl-quinazolin-4-yl]-1,4-diazepan-2-one (32.7 mg, 0.04 mmol) in dry THF (2 mL) was cooled to 0° C.

Tetrabutylammonium fluoride 1.OM in THF (0.08 mL, 0.08 mmol) was added and the reaction was allowed to warm to room temperature before stirring for 30 minutes. The reaction was concentrated to dryness. The residue was taken up in dry 1,4-Dioxane (2 mL). Hydrogen chloride (0.51 mL, 2.05 mmol) (4M in 1,4-dioxane) was added and the reaction was allowed to stir for 50 minutes. Silica was added and the reaction was concentrated to dryness. The product was purified by flash column chromatography on silica gel (4 g) eluting with 0-20% (1M ammonia in MeOH) in DCM. The desired fractions were united and concentrated to dryness to afford 4-[7-(8-ethynyl-7-fluoro-3-hydroxy-1-naphthyl)-2-[[rac-(2R,8S)-2-fluoro-1,2,3,5,6,7-hexahydropyrrolizin-8-yl]methoxy]-8-methyl-quinazolin-4-yl]-1,4-diazepan-2-one (11.5 mg, 47% yield) as a tan solid.

UPLC-MS (ES⁺, Method 1): 2.9 min, m/z 598.3 [M+H]⁺

¹H NMR (400 MHz, DMSO-d₆) δ/ppm 10.05 (s, 1H), 7.97-7.92 (m, 1H), 7.90 (d, J=8.6 Hz, 1H), 7.83-7.78 (m, 1H), 7.43 (t, J=9.0 Hz, 1H), 7.32 (d, J=2.6 Hz, 1H), 7.11 (d, J=8.6 Hz, 1H), 6.94 (d, J=2.5 Hz, 1H), 4.37 (d, J=4.7 Hz, 2H), 4.26-3.90 (m, 2H), 3.73 (s, 1H), 3.24-3.13 (m, 4H), 2.87 (s, 1H), 2.18-2.00 (m, 8H), 1.96-1.71 (m, 4H), 1.63-1.52 (m, 1H), 1.37-1.28 (m, 1H), 0.94 (t, J=7.3 Hz, 1H).

### Example 25

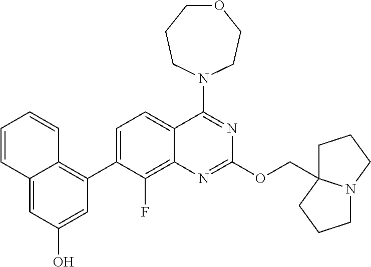 [SMILES: Oc1cc(-c2ccc3c(N4CCCOCC4)nc(OCC45CCCN4CCC5)nc3c2F)c2ccccc2c1]

### 4-[8-fluoro-2-(1,2,3,5,6,7-hexahydropyrrolizin-8-ylmethoxy)-4-(1,4-oxazepan-4-yl)quinazolin-7-yl]naphthalen-2-ol

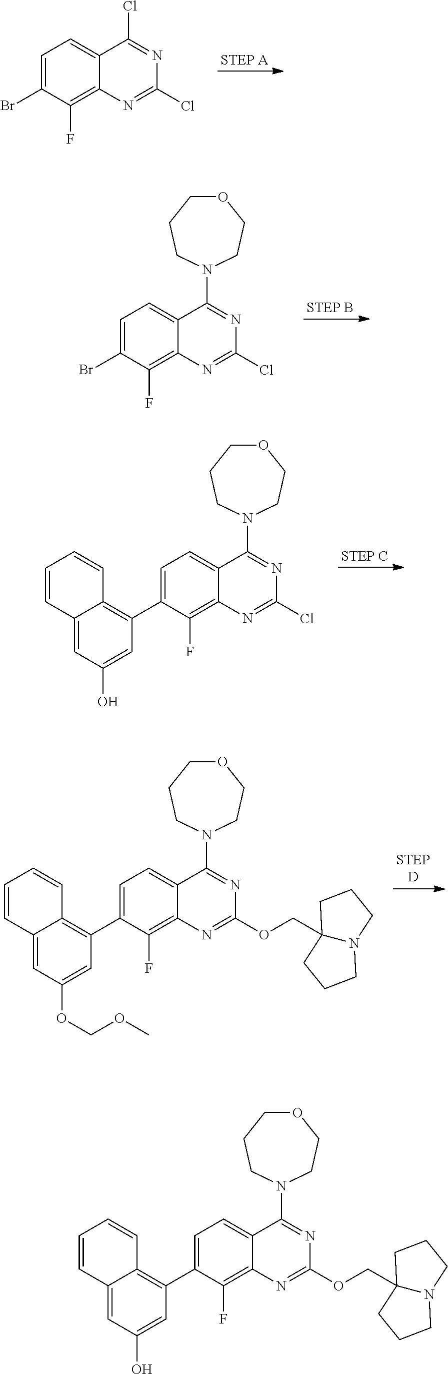 [SMILES: Fc1c(Br)ccc2c(Cl)nc(Cl)nc12 ; Fc1c(Br)ccc2c(N3CCCOCC3)nc(Cl)nc12 ; Oc1cc(-c2ccc3c(N4CCCOCC4)nc(Cl)nc3c2F)c2ccccc2c1 ; COCOc1cc(-c2ccc3c(N4CCCOCC4)nc(OCC45CCCN4CCC5)nc3c2F)c2ccccc2c1 ; Oc1cc(-c2ccc3c(N4CCCOCC4)nc(OCC45CCCN4CCC5)nc3c2F)c2ccccc2c1]

STEP A, 4-(7-bromo-2-chloro-8-fluoro-quinazolin-4-yl)-1,4-oxazepane. A mixture of 7-bromo-2,4-dichloro-8-fluoroquinazoline (500 mg, 1.69 mmol) and DIEA (1485.23 mg, 11.492 mmol) in DCM (10 ml) was stirred at −40° C. under N₂, before adding 1,4-oxazepane (279 mg, 2 mmol) and stirring at −40° C. under N₂ for 0.5 hours. The mixture was poured into water (100 mL), extracted with DCM (25 mL×3). The combined organic layers were washed with brine (50 mL), dried over Na₂SO₄ and concentrated under vacuum, The crude was purified by silica gel column chromatography (pet. ether/EtOAc=5/1 to pet.ether/EtOAc=2/1v/v) affording 4-(7-bromo-2-chloro-8-fluoro-quinazolin-4-yl)-1,4-oxazepane (500 mg, 82.04%) as a yellow solid.

UPLC-MS (ES⁺, Method 4): 1.7 min, m/z 361.9 [M+H]⁺

¹H NMR (400 MHz, DMSO-d₆) δ/ppm 7.89-7.86 (m, 1H), 7.70-7.66 (m, 1H), 4.09-4.01 (m, 4H), 3.90-3.86 (m, 2H), 3.77-3.68 (m, 2H), 2.11-2.01 (m, 2H).

STEP B, 4-[2-chloro-8-fluoro-7-[3-(methoxymethoxy)-1-naphthyl]quinazolin-4-yl]-1,4-oxazepane. A mixture of 7-bromo-2-chloro-8-fluoro-4-[1,4]oxazepan-4-yl-quinazoline (300 mg, 0.83 mmol), 2-(3-methoxymethoxy-naphthalen-1-yl)-4,4,5,5-tetramethyl-[1,3,2]dioxaborolane (261 mg, 0.83 mmol), Pd(dppf)Cl₂ (60.9 mg, 0.08 mmol) and K₂CO₃ (344 mg, 2.496 mmol) in 1,4-dioxane (4 mL) and H₂O (1 mL) was stirred at 60° C. under N₂ for 1.5 hours. The mixture concentrated under vacuum. The crude was purified by silica gel column (pet. ether/EtOAc=5/1 to pet. ether/EtOAc=2/1 v/v) to give 4-[2-chloro-8-fluoro-7-[3-(methoxymethoxy)-1-naphthyl]quinazolin-4-yl]-1,4-oxazepane (300 mg, 77%) as a yellow solid.

UPLC-MS (ES⁺, Method 4): 1.9 min, m/z 468.1 [M+H]⁺

¹H NMR (400 MHz, DMSO-d₆) δ/ppm 8.13-8.07 (m, 1H), 7.97-7.92 (m, 1H) 7.62-7.31 (m, 6H), 5.38 (s, 2H), 4.18-4.10 (m, 4H), 3.96-3.91 (m, 2H), 3.78-3.71 (m, 2H), 3.46 (s, 3H), 2.16-2.06 (m, 2H).

STEP C, 4-[8-fluoro-2-(1,2,3,5,6,7-hexahydropyrrolizin-8-ylmethoxy)-7-[3-(methoxymethoxy)-1-naphthyl]quinazolin-4-yl]-1,4-oxazepane. A mixture of (tetrahydro-pyrrolizin-7a-yl)-methanol (171 mg, 1.2 mmol) in DMF (8 ml) was stirred at 0° C. under N₂, before adding NaH (77 mg, 3.248 mmol) and stirring at 0° C. for 0.5 hours. 2-Chloro-8-fluoro-7-(3-methoxymethoxy-naphthalen-1-yl)-4-[1,4]oxazepan-4-yl-quinazoline (190 mg, 0.4 mmol) in DMF (2 mL) was added to the reaction solution. The mixture was warmed to 65° C. and stirred overnight. The mixture was poured into water (20 mL), extracted with EtOAc (20 mL×3).The combined organic layers were washed with brine (40 mL), dried over Na₂SO₄ and concentrated under vacuum affording 4-[8-fluoro-2-(1,2,3,5,6,7-hexahydropyrrolizin-8-ylmethoxy)-7-[3-(methoxymethoxy)-1-naphthyl]quinazolin-4-yl]-1,4-oxazepane (70 mg, 30%) as a yellow solid.

UPLC-MS (ES⁺, Method 4): 1.9 min, m/z 573.2 [M+H]⁺

¹H NMR (400 MHz, DMSO-d₆) δ/ppm 8.04-7.88 (m, 2H), 7.61-7.28 (m, 6H), 5.38 (s, 2H), 4.55 (s, 2H), 4.17-4.09 (m, 4H), 3.95-3.89 (m, 2H), 3.77-3.66 (m, 2H), 3.57-3.50 (m, 4H), 3.26-3.19 (m, 2H), 2.18-1.91 (m, 10H).

STEP D, 4-[8-fluoro-2-(1,2,3,5,6,7-hexahydropyrrolizin-8-ylmethoxy)-4-(1,4-oxazepan-4-yl)quinazolin-7-yl]naphthalen-2-ol. A mixture of 4-[8-fluoro-2-(1,2,3,5,6,7-hexahydropyrrolizin-8-ylmethoxy)-7-[3-(methoxymethoxy)-1-naphthyl]quinazolin-4-yl]-1,4-oxazepane (60 mg, 0.10 mmol) in HCl-1,4-dioxane (4M, 4 mL) was stirred at 25° C. under N₂ for 0.5 hours before concentrating to obtain 4-[8-fluoro-2-(1,2,3,5,6,7-hexahydropyrrolizin-8-ylmethoxy)-4-(1,4-oxazepan-4-yl)quinazolin-7-yl]naphthalen-2-ol (23.6 mg, 42%) as a white solid analysed as an HCl salt.

UPLC-MS (ES⁺, Method 4): 1.0 min, m/z 529.2 [M+H]⁺

¹H NMR (400 MHz, DMSO-d₆) δ/ppm 10.71 (s, 1H), 10.07 (s, 1H), 8.02 (d, J=8.8 Hz, 1H), 7.80 (d, J=8.3 Hz, 1H), 7.47-7.37 (m, 2H), 7.33-7.22 (m, 3H), 7.13 (d, J=2.4 Hz, 1H), 4.56 (s, 2H), 4.13 (m, 4H), 3.93 (m, 2H), 3.75 (m, 2H), 3.51 (m, 2H), 3.23-3.16 (m, 2H), 2.12 (m, 6H), 2.03-1.95 (m, 4H).

### Example 26

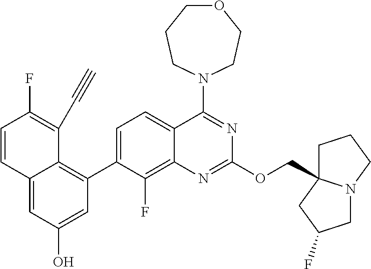 [SMILES: C#Cc1c(F)ccc2cc(O)cc(-c3ccc4c(N5CCCOCC5)nc(OC[C@@]56CCCN5C[C@H](F)C6)nc4c3F)c12]

### 5-ethynyl-6-fluoro-4-[8-fluoro-4-(1,4-oxazepan-4-yl)-2-[[rac-(2R,8S)-2-fluoro-1,2,3,5,6,7-hexahydropyrrolizin-8-yl]methoxy]quinazolin-7-yl]naphthalen-2-ol

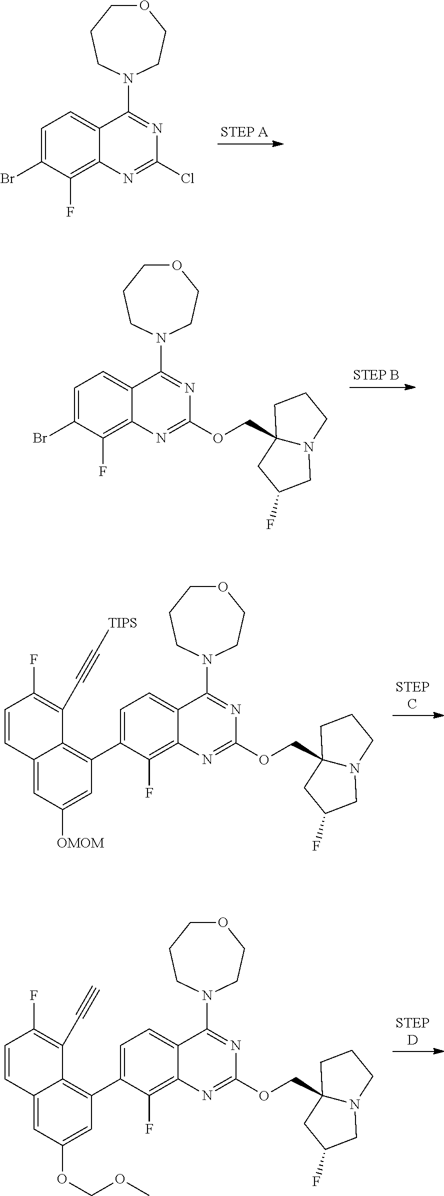

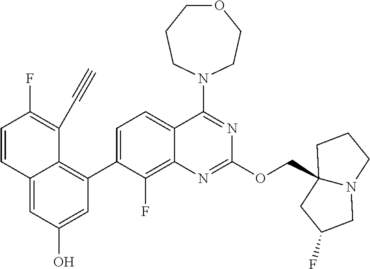 [SMILES: C#Cc1c(F)ccc2cc(O)cc(-c3ccc4c(N5CCCOCC5)nc(OC[C@@]56CCCN5C[C@H](F)C6)nc4c3F)c12]

STEP A, 4-[7-bromo-8-fluoro-2-[[rac-(2R,8S)-2-fluoro-1,2,3,5,6,7-hexahydropyrrolizin-8-yl]methoxy]quinazolin-4-yl]-1,4-oxazepane. A mixture of 7-bromo-2-chloro-8-fluoro-4-[1,4]oxazepan-4-yl-quinazoline (500 mg, 1.38 mmol) and (2-fluoro-tetrahydro-pyrrolizin-7a-yl)-methanol (220 mg, 1.38 mmol) in DMF (15 ml) was stirred at 0° C. under N₂,before adding NaH (266 mg, 11.1 mmol) was stirred at 0° C. for 0.5 hours. The reaction was left at room temperature and overnight. The mixture was poured into saturated aqueous NH₄Cl solution (150 mL), extracted with EtOAc (40 mL×3). The combined organic layers were washed with brine (60 mL), dried over Na₂SO₄ and concentrated. The crude was purified by silica gel column chromatography (DCM/MeOH=50/1 to DCM/MeOH=10/1, v/v) to give 4-[7-bromo-8-fluoro-2-[[rac-(2R,8S)-2-fluoro-1,2,3,5,6,7-hexahydropyrrolizin-8-yl]methoxy]quinazolin-4-yl]-1,4-oxazepane (550 mg, 82%) as a yellow solid.

UPLC-MS (ES⁺, Method 4): 0.9 min, m/z 485.1 [M+H]⁺

¹H NMR (400 MHz, DMSO-d₆) 5/ppm 7.80-7.74 (m, 1H), 7.47-7.41 (m, 1H), 4.09-3.98 (m, 4H), 3.82-3.79 (m, 2H), 3.74-3.71 (m, 2H), 3.60-3.51 (m, 4H), 3.11-3.07 (m, 2H), 2.86-2.81 (m, 1H), 2.06-1.77 (m, 6H), 1.48-1.44 (m, 2H).

STEP B, 2-[2-fluoro-8-[8-fluoro-4-(1,4-oxazepan-4-yl)-2-[[rac-(2R,8S)-2-fluoro-1,2,3,5,6,7-hexahydropyrrolizin-8-yl]methoxy]quinazolin-7-yl]-6-(methoxymethoxy)-1-naphthyl]ethynyl-triisopropyl-silane. 4-[7-bromo-8-fluoro-2-[[rac-(2R,8S)-2-fluoro-1,2,3,5,6,7-hexahydropyrrolizin-8-yl]methoxy]quinazolin-4-yl]-1,4-oxazepane (200 mg, 0.414 mmol), 2-{7-Fluoro-3-methoxymethoxy-8-[(triisopropylsilanyl)-ethynyl]-naphthalen-1-yl}-4,4,5,5-tetramethyl-[1,3,2]dioxaborolane (318 mg, 0.62 mmol), Pd(dtpbf)Cl₂ (26.7 mg, 0.04 mmol) and Cs₂CO₃ (269.6 mg, 0.82 mmol) in 3:11,4-dioxane/H₂O=(20 mL) was stirred at 50° C. under N₂ for 2 hours. The mixture was concentrated in vacuum and purified by silica gel column chromatography (DCM/MeOH=50/1 to DCM/MeOH=30/1, v/v) to give 2-[2-fluoro-8-[8-fluoro-4-(1,4-oxazepan-4-yl)-2-[[rac-(2R,8S)-2-fluoro-1,2,3,5,6,7-hexahydropyrrolizin-8-yl]methoxy]quinazolin-7-yl]-6-(methoxymethoxy)-1-naphthyl]ethynyl-triisopropyl-silane (120 mg, 37%) as a yellow solid.

UPLC-MS (ES⁺, Method 4): 1.9 min, m/z 789.3 [M+H]⁺

STEP C, 4-[7-[8-ethynyl-7-fluoro-3-(methoxymethoxy)-1-naphthyl]-8-fluoro-2-[[rac-(2R,8S)-2-fluoro-1,2,3,5,6,7-hexahydropyrrolizin-8-yl]methoxy]quinazolin-4-yl]-1,4-oxazepane. A mixture of 2-[2-fluoro-8-[8-fluoro-4-(1,4-oxazepan-4-yl)-2-[[rac-(2R,8S)-2-fluoro-1,2,3,5,6,7-hexahydropyrrolizin-8-yl]methoxy]quinazolin-7-yl]-6-(methoxymethoxy)-1-naphthyl]ethynyl-triisopropyl-silane (110 mg, 0.139 mmol) and CsF (105.5 mg, 0.69 mmol) in DMF (10 mL) was stirred at 25° C. for 1 hour. The mixture was poured into water (100 mL), extracted with EtOAc (25 mL×3). The combined organic layers were washed with brine (50 mL), dried over Na₂SO₄ and concentrated to give crude 4-[7-[8-ethynyl-7-fluoro-3-(methoxymethoxy)-1-naphthyl]-8-fluoro-2-[[rac-(2R,8S)-2-fluoro-1,2,3,5,6,7-hexahydropyrrolizin-8-yl]methoxy]quinazolin-4-yl]-1,4-oxazepane (100 mg, 90.69%) as a dark solid taken directly into the next step as a crude mixture.

UPLC-MS (ES⁺, Method 4): 1.3 min, m/z 633.1 [M+H]⁺

STEP D, 5-ethynyl-6-fluoro-4-[8-fluoro-4-(1,4-oxazepan-4-yl)-2-[[rac-(2R,8S)-2-fluoro-1,2,3,5,6,7-hexahydropyrrolizin-8-yl]methoxy]quinazolin-7-yl]naphthalen-2-ol. A mixture of 4-[7-[8-ethynyl-7-fluoro-3-(methoxymethoxy)-1-naphthyl]-8-fluoro-2-[[rac-(2R,8S)-2-fluoro-1,2,3,5,6,7-hexahydropyrrolizin-8-yl]methoxy]quinazolin-4-yl]-1,4-oxazepane (90 mg, 0.14 mmol) in HCl-dioxane (4M, 5 mL) was stirred at room temperature for 0.5 hours. The mixture was concentrated under vacuum and subject to preparative HPLC purification to give 5-ethynyl-6-fluoro-4-[8-fluoro-4-(1,4-oxazepan-4-yl)-2-[[rac-(2R,8S)-2-fluoro-1,2,3,5,6,7-hexahydropyrrolizin-8-yl]methoxy]quinazolin-7-yl]naphthalen-2-ol (23 mg, 28%) as a yellow solid.

UPLC-MS (ES⁺, Method 4): 1.8 min, m/z 589.1 [M+H]⁺

¹H NMR (400 MHz, DMSO-d₆) δ/ppm 7.97-7.93 (m, 1H), 7.82 (d, J=8.8 Hz, 1H), 7.47-7.43 (m, 1H), 7.35 (s, 1H), 7.19-7.16 (m, 1H), 7.06 (s, 1H), 5.28 (d, J=54.1 Hz, 1H), 4.11-3.99 (m, 6H), 3.90 (d, J=9.0 Hz, 2H), 3.76 (s, 2H), 3.15-2.99 (m, 4H), 2.86-2.82 (m, 1H), 2.18-1.69 (m, 8H).

### Example 27

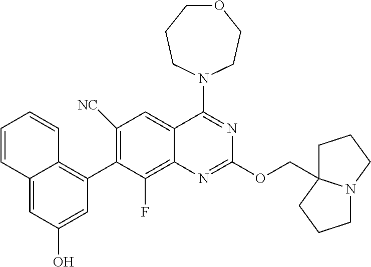 [SMILES: N#Cc1cc2c(N3CCCOCC3)nc(OCC34CCCN3CCC4)nc2c(F)c1-c1cc(O)cc2ccccc12]

### 8-fluoro-2-(1,2,3,5,6,7-hexahydropyrrolizin-8-ylmethoxy)-7-(3-hydroxy-1-naphthyl)-4-(1,4-oxazepan-4-yl)quinazoline-6-carbonitrile

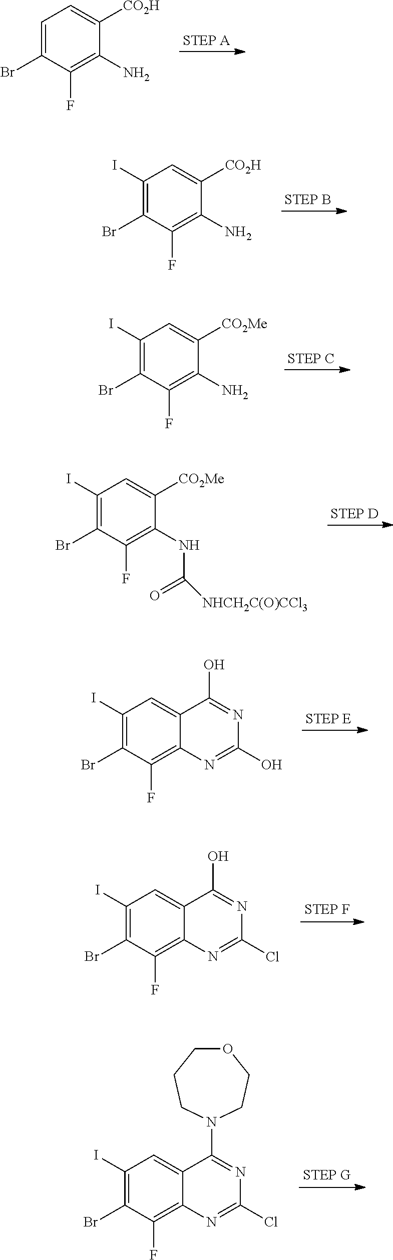

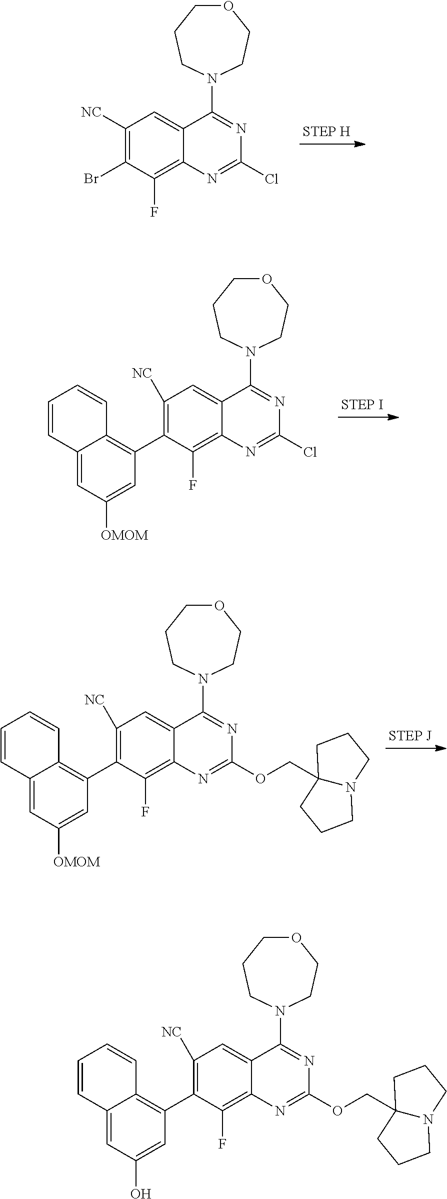

STEP A, 2-amino-4-bromo-3-fluoro-5-iodo-benzoic acid. To a solution of 2-Amino-4-bromo-3-fluorobenzoic acid (4.84 g, 20.68 mmol) in DMF (20 mL) was added N-iodosuccinimide (6.98 g, 31.02 mmol) at r.t. The mixture was stirred at 80° C. for 3 h, cooled to r.t and poured into water (200 mL). The fine precipitate was collected by filtration. The precipitate in the filter was washed with DCM/MeOH. The solution was reduced in vacuo. The dark solid was triturated with 20 mL, collected by vacuum filtration and dried under vacuum at 50° C. affording 2-amino-4-bromo-3-fluoro-5-iodo-benzoic acid as am orange solid (5.8 g, 78%).

UPLC-MS (ES⁺, Method 2): 1.8 min, m/z 359.9/361.8 [M+H]⁺

¹H NMR (400 MHz, DMSO-d₆) δ/ppm 13.36 (bs, 1H), 7.99 (s, 1H), 6.90 (bs, 2H).

STEP B, methyl 2-amino-4-bromo-3-fluoro-5-iodo-benzoate. To a solution of 2-amino-4-bromo-3-fluoro-5-iodo-benzoic acid (50 mg, 0.14 mmol) in DMF (0.5 mL) was added cesium carbonate (67.89 mg, 0.21 mmol). The suspension was stirred at r.t. for 2 h before addition of iodomethane (8.65 uL, 0.14 mmol) diluted in DMF (0.5 mL). The mixture was stirred at r.t. over the weekend before pouring into brine. The aqueous layer was extracted with EtOAc (x4). The combined organics were washed with brine, dried with Na₂SO₄ and reduced in vacuo affording methyl 2-amino-4-bromo-3-fluoro-5-iodo-benzoate as a red solid (41 mg, 80%).

UPLC-MS (ES⁺, Method 2): 2.1 min, m/z 373.9/375.9 [M+H]⁺

¹H NMR (400 MHz, DMSO-d₆) δ/ppm 7.98 (d, J=1.8 Hz, 1H), 6.86 (bs, 2H), 3.83 (s, 3H).

STEP C, methyl 4-bromo-3-fluoro-5-iodo-2-(trichloromethylcarbamoylamino)benzoate. To a stirred solution of methyl 2-amino-4-bromo-3-fluoro-5-iodo-benzoate (2000 mg, 5.35 mmol) in THF (50 mL) at r.t. was added trichloroacetyl isocyanate (0.7 mL, 5.88 mmol) dropwise.

After 4 hrs the volatiles were removed in vacuo. The residue was titurated with Et₂O affording methyl 4-bromo-3-fluoro-5-iodo-2-(trichloromethylcarbamoylamino)benzoate (2.8 g, 100%), as an off-white solid.

UPLC-MS (ES⁺, Method 2): 1.6 min, m/z 416.9/418.9 [M+H]⁺

¹H NMR (400 MHz, DMSO-d₆) δ/ppm 1.83 (bs, 1H), 10.03 (s, 1H), 8.17 (d, J=1.7 Hz, 1H), 3.82 (s, 3H).

STEP D, 7-bromo-8-fluoro-6-iodo-quinazoline-2,4-diol. 7M Ammonia in MeOH (3.63 mL, 25.43 mmol) was added to a suspension of methyl 4-bromo-3-fluoro-5-iodo-2-[(2,2,2-trichloroacetyl)carbamoylamino]benzoate (2860 mg, 5.09 mmol) in methanol (50 mL) at room temperature and allowed to stir at room temperature for 30 mins. Afterwards, all volatiles were removed under reduced pressure. The residue was triturated with diethyl ether, 7-bromo-8-fluoro-6-iodo-quinazoline-2,4-diol was collected by filtration as a white solid and dried under reduced pressure (1.9 g, 100%).

UPLC-MS (ES⁺, Method 2): 1.6 min, m/z 382.8/384.8 [M−H]⁺

¹H NMR (400 MHz, DMSO-d₆) δ/ppm 8.27 (bs, 2H),8.07 (d, J=1.6 Hz, 1H) ppm.

STEP E, 7-bromo-2-chloro-8-fluoro-6-iodo-quinazolin-4-ol. A suspension of 7-bromo-8-fluoro-6-iodo-quinazoline-2,4-diol (954 mg, 2.48 mmol) in phosphorus oxychloride (6.93 mL, 74.35 mmol) was heated to 100° C. followed by addition of N,N-Diisopropylethylamine (1.08 mL, 6.2 mmol). The mixture was stirred at 100° C. for 2 h and cooled down. The mixture was poured into a stirred solution of NaHCO₃ (aq, sat, 500 mL) and extracted with DCM (x4).

The combined organics were washed with brine, dried with Na₂SO₄ and reduced in vacuo affording 7-bromo-2-chloro-8-fluoro-6-iodo-quinazolin-4-ol (868 mg) as an orange solid used without purification.

UPLC-MS (ES⁺, Method 2): 2.39 min, m/z 400.7/402.8/404.7 [M−H]⁺

STEP F, 4-(7-bromo-2-chloro-8-fluoro-6-iodo-quinazolin-4-yl)-1,4-oxazepane. Under an N₂-atmosphere on an ice-bath a mixture of HATU (2 g, 5.34 mmol), and 7-bromo-2-chloro-8-fluoro-6-iodo-quinazolin-4-ol (1077 mg, 2.67 mmol) in DMA (13 mL) was added N,N-diisopropylethylamine (2.79 mL, 16.02 mmol). After 30 min [1,4]-oxazepane (0.59 mL, 5.34 mmol) was added at 0° C. The ice-bath was removed and stirring continued at r.t. After 2 hrs the mixture was diluted with DCM and washed with brine. The aqueous layer was extracted with DCM (x3), passed through a phase separator and reduced in vacuo. The residue was dry loaded to flash chromatography (SiO₂, 12 g, EtOAc in petroleum ether 0-100%) and residual solvent was removed by co-distillation with toluene (x4). After evaporation of product containing fractions and further drying under vacuum 4-(7-bromo-2-chloro-8-fluoro-6-iodo-quinazolin-4-yl)-1,4-oxazepane was obtained 955 mg, 74%) as an orange solid.

UPLC-MS (ES⁺, Method 2): 2.2 min, m/z 485.8/487.8/489.8 [M+H]⁺

¹H NMR (400 MHz, CDCl₃) δ/ppm 8.26 (d, J=2.0 Hz, 1H), 4.13-4.05 (m, 4H), 3.98 (dd, J=5.8, 3.3 Hz, 2H), 3.89-3.79 (m, 2H), 2.20 (m, 2H).

STEP G, 7-bromo-2-chloro-8-fluoro-4-(1,4-oxazepan-4-yl)quinazoline-6-carbonitrile. A mixture of 4-(7-bromo-2-chloro-8-fluoro-6-iodo-quinazolin-4-yl)-1,4-oxazepane (887 mg, 1.82 mmol) and copper cyanide (326.58 mg, 3.65 mmol) in DMF (9 mL) was purged with N₂ for 15 mins. The mixture was stirred under N₂ at 100° C. After 4.5 h the mixture was cooled, diluted with EtOAc and washed with brine. The aqueous layer was extracted with EtOAc (x2) and the combined organics were washed with a minimum amount of brine. The organics were dried with Na₂SO₄, reduced in vacuo and the residual DMF was co-distilled with toluene (x2). The crude product was dry-loaded to flash chromatography (SiO₂, 12 g, EtOAc in petroleum ether 0-70%), the desired fractions were concentrated to afford 7-bromo-2-chloro-8-fluoro-4-(1,4-oxazepan-4-yl)quinazoline-6-carbonitrile (360 mg, 51%) as a yellow solid.

UPLC-MS (ES⁺, Method 2): 1.9 min, m/z 385.0/387.0 [M+H]⁺

¹H NMR (400 MHz, CDCl₃) δ/ppm 8.52 (d, J=1.7 Hz, 1H), 4.15-4.07 (m, 4H), 3.91-3.86 (m, 2H), 3.70 (m, 2H), 2.04 (m, 2H).

STEP H, 2-chloro-8-fluoro-7-[3-(methoxymethoxy)-1-naphthyl]-4-(1,4-oxazepan-4-yl)quinazoline-6-carbonitrile. A mixture of 2-(3-(2-methoxymethoxy)naphthalene-1-yl)-4,4,5,5-tetramethyl-1,3,2-dioxaborolane (325.16 mg, 1.03 mmol), 7-bromo-2-chloro-8-fluoro-4-(1,4-oxazepan-4-yl)quinazoline-6-carbonitrile (307 mg, 0.8 mmol), cesium carbonate (518.78 mg, 1.59 mmol) and [1,1′-Bis(diphenylphosphino)ferrocene]Palladium(II) chloride dichloromethane complex (130.03 mg, 0.16 mmol) were suspended in 1,4-dioxane (7 mL) and 1,4-dioxane (7 mL). The vial was sealed and purged with N₂ for 15 min. The mixture was irradiated in a microwave to an internal temperature of 100° C. After 2 hrs the resulting mixture was colled and filtered before concentrating. The residue was dry-loaded to flash chromatography (SiO₂, 4 g, EtOAc in petroleum ether 0-100%), product containing fractions were concentrated to afford 2-chloro-8-fluoro-7-[3-(methoxymethoxy)-1-naphthyl]-4-(1,4-oxazepan-4-yl)quinazoline-6-carbonitrile (136 mg, 22%) as a yellow solid.

UPLC-MS (ES⁺, Method 2): 2.1 min, m/z 493.1/495.0 [M+H]⁺

¹H NMR (400 MHz, DMSO-d₆) δ/ppm 8.62 (d, J=0.1 Hz, 1H), 7.99 (d, J=8.2 Hz, 1H), 7.70 (d, =2.5 Hz, 1H),7.60-7.54 (m, 1H), 7.43 (d, J=2.5 Hz, 1H), 7.41-7.37 (m, 2H), 5.42 (s, 2H), 4.25-4.15 (m, 4H), 3.96-3.92 (m, 2H), 3.77-3.70 (m, 2H), 3.47 (s, 3H), 2.14-2.02 (m, 2H).

STEP I, 8-fluoro-2-(1,2,3,5,6,7-hexahydropyrrolizin-8-ylmethoxy)-7-[3-(methoxymethoxy)-1-naphthyl]-4-(1,4-oxazepan-4-yl)quinazoline-6-carbonitrile. In a sealed vial a solution of 2-chloro-8-fluoro-7-[3-(methoxymethoxy)-1-naphthyl]-4-(1,4-oxazepan-4-yl)quinazoline-6-carbonitrile (110 mg, 0.22 mmol), (tetrahydro-1H-pyrrolizin-7a(5H)-yl)methanol (126.05 mg, 0.89 mmol) and N,N-diisopropylethylamine (0.23 mL, 1.34 mmol) in dry 1,4-dioxane (1 mL) was heated to 100° C. After 8 hrs the temperature was reduced to 70° C. and stirring continued overnight. The mixture was cooled to r.t. and volatiles were removed in vacuo and the mixture was dry-loaded on Celite to reverse phase chromatography (SiO₂—C18, 4 g, MeCN in H₂O, 0-100%, with 0.1% (NH₄)₂CO₃). The desired fractions were concentrated to afford 8-fluoro-2-(1,2,3,5,6,7-hexahydropyrrolizin-8-ylmethoxy)-7-[3-(methoxymethoxy)-1-naphthyl]-4-(1,4-oxazepan-4-yl)quinazoline-6-carbonitrile (56 mg, 42%) as a yellow solid.

UPLC-MS (ES⁺, Method 2): 1.7 min, m/z 598.3 [M+H]⁺

¹H NMR (400 MHz, DMSO-d₆) δ/ppm 8.50 (s, 1H), 7.98 (d, J=8.4 Hz, 1H), 7.68 (d, J=2.4 Hz, 1H), 7.57 (ddd, J=3.6, 4.6, 8.3 Hz, 1H), 7.40 (d, J=2.5 Hz, 1H), 7.38 (d, J=4.0 Hz, 1H), 5.40 (s, 2H), 4.21-4.12 (m, 4H), 3.94 (m, 2H), 3.74 (m, 2H), 3.47 (s, 3H), 2.68 (m, 2H).

STEP J, 8-fluoro-2-(1,2,3,5,6,7-hexahydropyrrolizin-8-ylmethoxy)-7-(3-hydroxy-1-naphthyl)-4-(1,4-oxazepan-4-yl)quinazoline-6-carbonitrile. To a solution of 8-fluoro-2-(1,2,3,5,6,7-hexahydropyrrolizin-8-ylmethoxy)-7-[3-(methoxymethoxy)-1-naphthyl]-4-(1,4-oxazepan-4-yl)quinazoline-6-carbonitrile (54 mg, 0.09 mmol) and triethylsilane (0.04 mL, 0.27 mmol) in DCM (1.8 mL) was added trifluoroacetic acid (0.21 mL, 2.71 mmol). The mixture was stirred at r.t. After 90 mins all volatiles were removed and the residue was dry-loaded on celite to reverse phase chromatography (C18-SiO₂, 4 g, MeCN in H₂O, 0-100%, with 0.1% (NH₄)₂CO₃) affording 8-fluoro-2-(1,2,3,5,6,7-hexahydropyrrolizin-8-ylmethoxy)-7-(3-hydroxy-1-naphthyl)-4-(1,4-oxazepan-4-yl)quinazoline-6-carbonitrile (25 mg, 50%) as a yellow solid.

UPLC-MS (ES⁺, Method 1): 3.0 min, m/z 554.4 [M+H]⁺

¹H NMR (400 MHz, DMSO-d₆) δ/ppm 10.10 (s, 1H), 8.49 (s, 1H), 7.85 (d, J=8.4 Hz, 1H), 7.48 (ddd, J=1.8, 6.3, 8.1 Hz, 1H), 7.34 (d, J=2.3 Hz, 1H), 7.30 (d, J=7.8 Hz, 1H), 7.26 (dt, J=1.1, 7.3 Hz, 1H), 7.17 (d, J=2.4 Hz, 1H), 4.20-4.10 (m, 6H), 3.93 (m, 2H), 3.74 (m, 2H), 3.01 (m, 1H), 2.56-2.51 (m, 2H), 2.09 (m, 2H), 1.98-1.88 (m, 2H), 1.88-1.72 (m, 4H), 1.70-1.56 (m, 2H).

### Biological Results

### HTRF Nucleotide Exchange Assay Method

The capacity of compounds to bind KRAS G12D, other KRAS mutants and wildtype RAS isoforms was quantified using a HTRF nucleotide exchange assay. Recombinant human RAS protein (2 nM; aa1-188 KRAS WT, HRAS WT, NRAS WT, or KRAS containing the containing the G12D, G13D or Q61H amino acid substitutions, or 4 nM KRAS; aa1-188 containing the G12V, G12C, G12A or G12S amino acid substitution, an N-terminal 6×His-tag and leader sequence), and 2 nM Europium-labeled anti-6×His antibody were mixed in assay buffer (10 mM HEPES pH7.3, 150 mM NaCl, 5 mM MgCl₂, 0.05% BSA, 0.0025% NP-40 and 100 mM KF) with various concentrations of compound in a 384-well plate and a volume of 5 uL. After a 60-minute incubation at room temperature, Sul of 200 nM EDA-GTP-DY647P1 (diluted in assay buffer) was added to the plate. Following 30-minute incubation at room temperature, time-resolved fluorescence was measured on a PerkinElmer Envision plate reader. DMSO (0.3%) and unlabeled GDP (1 μM) or equivalent tool compound were used to generate the Max and Min assay signals, respectively. Data was analysed using a four-parameter logistic model to calculate IC₅₀ values, with at least two independent replicates were performed for each compound. The results are presented as IC₅₀s in Table 5 where “A” corresponds to an IC₅₀<10 nM, “B” to an IC₅₀>10 nM up to 100 nM, “C” is >100 nM up to 10 μM, “D” represents >10% up to 49% inhibition at 10 μM and ND=not determined:

| | KRAS^{G12D} | KRAS^{WT} | KRAS^{G12C} | KRAS^{G12V} | KRAS^{G12S} | KRAS^{G12A} | KRAS^{G13D} | KRAS^{Q61H} |
|---|---|---|---|---|---|---|---|---|
| Example | NEA | NEA | NEA | NEA | NEA | NEA | NEA | NEA |
| 1 | A | C | C | ND | ND | ND | D | ND |
| 2 | C | C | C | C | ND | ND | D | C |
| 3 | C | C | C | ND | ND | ND | C | ND |
| 4 | D | D | ND | ND | ND | ND | D | ND |
| 5 | D | D | ND | ND | ND | ND | D | ND |
| 6 | A | A | A | A | A | A | D | A |
| 7 | D | D | ND | ND | D | ND | ND | ND |
| 8 | D | D | ND | ND | D | ND | ND | ND |
| 9 | D | D | ND | ND | D | ND | ND | ND |
| 10 | D | D | ND | ND | ND | ND | ND | ND |
| 11 | D | D | ND | ND | ND | ND | ND | ND |
| 12 | D | D | ND | ND | ND | ND | ND | ND |
| 13 | C | C | ND | ND | ND | ND | ND | ND |
| 14 | D | D | ND | ND | ND | ND | ND | ND |
| 15 | D | D | ND | ND | ND | ND | ND | ND |
| 16 | D | C | ND | ND | ND | ND | ND | ND |
| 17 | D | D | ND | ND | ND | ND | ND | ND |
| 18 | D | D | ND | ND | ND | ND | ND | ND |
| 19 | D | D | ND | ND | ND | ND | ND | ND |
| 20 | D | D | ND | ND | ND | ND | ND | ND |
| 21 | D | D | ND | ND | ND | ND | ND | ND |
| 22 | B | B | A | A | C | B | ND | B |
| 23 | D | D | ND | ND | ND | ND | ND | ND |
| 24 | A | A | A | A | B | A | ND | B |
| 25 | B | B | B | B | C | B | D | C |
| 26 | A | A | A | A | A | A | ND | ND |
| 27 | B | B | B | B | C | C | ND | ND |

As can be seen from Table 5, compounds of the invention exhibit KRAS inhibition across a broad spectrum of KRAS proteins, including wild-type KRAS and KRAS having a range of mutations.

1. A compound of formula (Ia), or a pharmaceutically acceptable salt thereof: 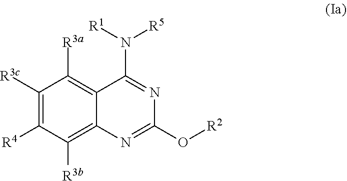
wherein
R¹ is independently selected from C₀-C₃-alkylene-R^{1a} and C₂-C₆-alkylene-R^{1b}; wherein R^{1a} is independently selected from a 4- to 7-membered heterocycloalkyl ring; a fused or spirofused bicyclic 6- to 11-membered heterocycloalkyl group, optionally substituted with from 1 to 4 R⁹ groups and a C₃-C₇-cycloalkyl ring; wherein said heterocycloalkyl ring or said cycloalkyl ring is optionally substituted with from 1 to 4 R⁹ groups; R^{1b} is independently selected from: OR⁸, SR⁸, SOR⁸, SO₂R⁸, SO(NH)R⁸, OC(O)R⁸, and SO₂NR⁷R⁸;
or R¹ and R⁵ together with the nitrogen to which they are attached form a ring system selected from: a monocyclic 4- to 7-membered heterocycloalkyl group, optionally substituted with from 1 to 4 R⁹ groups; a fused, spirofused or bridged bicyclic 6- to 11-membered heterocycloalkyl group, optionally substituted with from 1 to 4 R⁹ groups;
wherein R¹ and R⁵ are selected such that NR¹R⁵ comprises no more than a single amine, wherein said single amine may be a primary, secondary or tertiary amine;
R² is independently C₁-C₆-alkyl, C₁-C₄-haloalkyl, C₀-C₄-alkylene-R^{2a}, C₁-C₄-alkylene-R^{2b}, C₂-C₄-alkylene-R^{2c};
R^{2a} is independently selected from monocyclic 4- to 7-membered heterocycloalkyl group, a fused, spirofused or bridged bicyclic 6- to 11-membered heterocycloalkyl group; a 5-, 6-, 9- or 10-membered monocyclic or bicyclic heteroaryl group; phenyl; C₃-C₇-cycloalkyl; wherein any heterocycloalkyl or cycloalkyl R^{2a} group is optionally substituted with from 1 to 6 R¹⁰ groups and any heteroaryl or phenyl R^{2a} group is optionally substituted with from 1 to 6 R¹¹ groups;
R^{2b} is independently selected from CONR¹²R¹² and CO₂R¹²;
R^{2b} is independently selected from NR¹²R¹³ and OR¹²
or R² and R⁶ together with the nitrogen to which they are attached form a ring system selected from: monocyclic 4- to 7-membered heterocycloalkyl group, a fused, spirofused or bridged bicyclic 6- to 11-membered heterocycloalkyl group, said heterocycloalkyl group being optionally substituted with from 1 to 6 R¹⁰ groups;
R^{3a}, R^{3b} and R^{3c} are each independently selected from: H, halo, C₁-C₄-alkyl, O—C₁-C₄-alkyl, C₁-C₄-haloalkyl, O—C₁-C₄-haloalkyl, cyclopropyl, nitro and cyano;
R⁴ is independently selected from: phenyl, said phenyl being optionally fused to a C₅-C₇-cycloalkyl ring; naphthyl; and 5-, 6-, 9- or 10-membered monocyclic or bicyclic heteroaryl, wherein R⁴ is optionally substituted with from 1 to 4 R¹⁴ groups;
R⁵, R⁶, R⁸ and R¹² are each independently selected at each occurrence from H, C₁-C₄-haloalkyl, and C₁-C₄-alkyl;
R⁷ and R¹³ are each independently at each occurrence selected from H, C₁-C₄-alkyl, C₁-C₄-haloalkyl and C(O)—C₁-C₄-alkyl;
R⁹ and R¹⁰ are each independently at each occurrence selected from oxo, halo, cyano, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₁-C₄-alkyl substituted with cyano, C₂-C₄-alkenyl, C₂-C₄-alkynyl, C₁-C₄-haloalkyl and cyclopropyl;
R¹¹ and R¹⁴ are each independently at each occurrence selected from halo, cyano, nitro, NR¹²R¹³, OR¹², CO₂R¹², CONR¹²R¹², C₁-C₄-alkyl, C₁-C₄-alkyl substituted with NR¹²R¹³, C₁-C₄-alkyl substituted with OR¹², C₂-C₄-alkenyl, C₂-C₄-alkynyl, C₁-C₄-haloalkyl and cyclopropyl;
wherein any of the aforementioned alkyl, alkylene or cyclopropyl groups is optionally substituted, where chemically possible, by 1 to 5 substituents which are each independently at each occurrence selected from the group consisting of: C₁-C₄-alkyl, halo, nitro, cyano, NR^{a}R^{b}, OR^{a}, SR^{a}, CO₂R^{a}, C(O)R^{a}, CONR^{a}R^{a}; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.

2. A compound of claim 1, wherein R¹ and R⁵ together with the nitrogen to which they are attached form a ring system selected from: a monocyclic 4- to 7-membered heterocycloalkyl group, optionally substituted with from 1 to 4 R⁹ groups; a fused or spirofused bicyclic 6- to 11-membered heterocycloalkyl group, optionally substituted with from 1 to 4 R⁹ groups; and a bridged bicyclic 6- to 11-membered heterocycloalkyl group, optionally substituted with from 1 to 4 R⁹ groups; wherein the nitrogen to which R¹ and R⁵ are attached is the only nitrogen in the ring system.

3. A compound of claim 1, wherein R¹ and R⁵ are selected such that the nitrogen of NR¹R⁵ is the nitrogen single amine.

4. A compound of any one of claims 1 to 3, wherein R¹ is C₀-C₃-alkylene-R^{1a} wherein R^{1a} is independently selected from a 4- to 7-membered heterocycloalkyl ring; and a C₃-C₇-cycloalkyl ring substituted with an NR⁷R⁸ group; wherein said heterocycloalkyl ring or said cycloalkyl ring is optionally substituted with from 1 to 4 R⁹ groups.

5. A compound of any one of claims 1 to 3, wherein R¹ and R⁵ together with the nitrogen to which they are attached form a ring system selected from: a monocyclic 4- to 7-membered group heterocycloalkyl group, optionally substituted with from 1 to 4 R⁹ groups; a fused or spirofused bicyclic 6- to 11-membered heterocycloalkyl group, optionally substituted with from 1 to 4 R⁹ groups.

6. A compound of claim 5, wherein R¹ and R⁵ together with the nitrogen to which they are attached form a ring system having the structure: 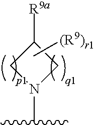
wherein R^{9a} is selected from NR¹²R¹³ and C₁-C₄-alkyl substituted with NR¹²R¹³; p1 is selected from 0, 1, 2 and 3, q1 is selected from 0, 1 and 2; and r1 is selected from 0, 1, 2 and 3.

7. A compound of claim 5, wherein R¹ and R⁵ together with the nitrogen to which they are attached form a ring system having the structure: 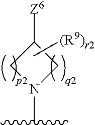
wherein Z⁶ is independently selected from C(O)NR^{9b}, NR^{9b}, O, S, S(O)₂,S(O), S(O)(NR^{9b}) and S(O)(NH); R^{9b} is selected from H and C₁-C₄-alkyl; p2 is selected from 2 and 3, q2 is 2; and r2 is selected from 0, 1, 2 and 3.

8. A compound of claim 5, wherein R¹ and R⁵ together with the nitrogen to which they are attached form a bridged bicyclic 6- to 11-membered heterocycloalkyl group, optionally substituted with from 1 to 4 R⁹ groups.

9. A compound of any one of claims 1 to 8, wherein R² has the structure: 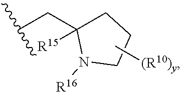
wherein R¹⁵ is independently selected from H, C₁-C₄-alkyl; wherein R¹⁶ is independently selected from H, C₁-C₄-alkyl and cyclopropyl; or wherein R¹⁵ and R¹⁶ together with the atoms to which they are attached form a 5- or 6-membered heterocycloalkyl ring, optionally substituted with 1 or 2 R¹⁰ groups; and y is independently selected from 0, 1, 2, 3, and 4.

10. A compound of claim 9, wherein R² has the structure: 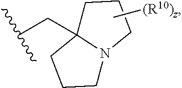
wherein z is independently selected from 0, 1, 2, 3, and 4.

11. A compound of any one of claims 1 to 10, wherein R^{3b} is F.

12. A compound of any one of claims 1 to 11, wherein both R^{3a} and R^{3c} are H.

13. A compound of any one of claims 1 to 12, wherein R⁴ is phenyl, said phenyl being optionally fused to a C₅-C₇-cycloalkyl ring, wherein R⁴ is optionally substituted with from 1 to 4 R⁴ groups.

14. A compound of any one of claims 1 to 12, wherein R⁴ has the structure: 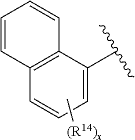
wherein x is independently selected from 0, 1, 2, 3, and 4.

15. A compound of claim 14, wherein R⁴ has the structure: 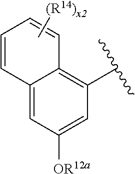
wherein R^{12a} is independently H or C₁-C₄-alkyl; x2 is independently selected from 0, 1, 2 and 3.

16. A compound of any one of claims 1 to 12, wherein R⁴ is 5-, 6-, 9- or 10-membered monocyclic or bicyclic heteroaryl, optionally substituted with from 1 to 4 R¹⁴ groups.

17. A compound of claim 1, wherein the compound of formula (I) is selected from:

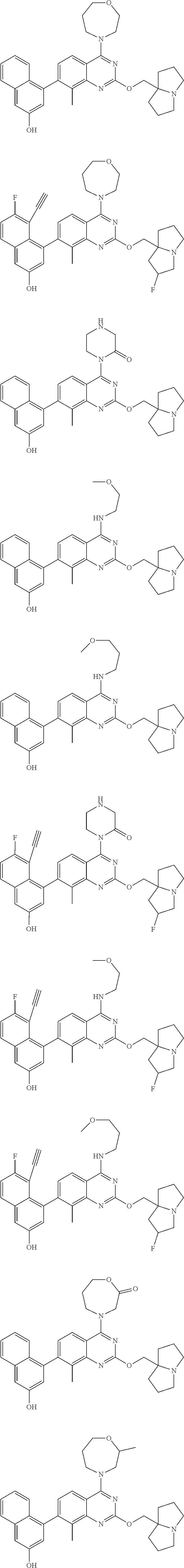

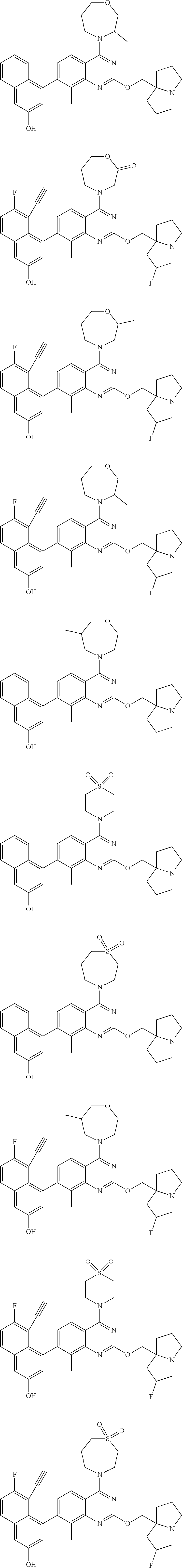

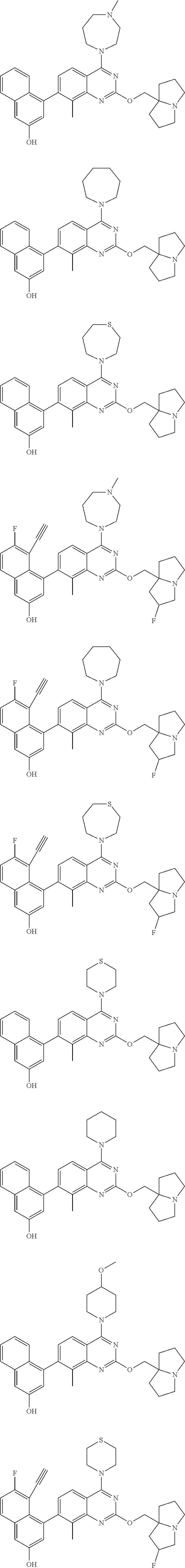

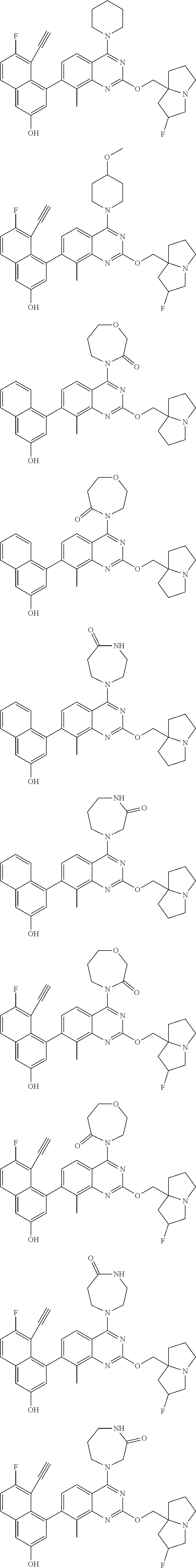

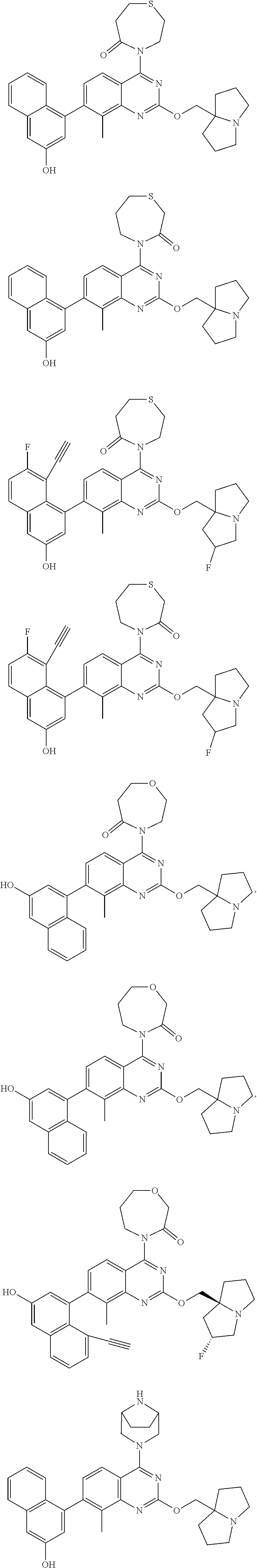

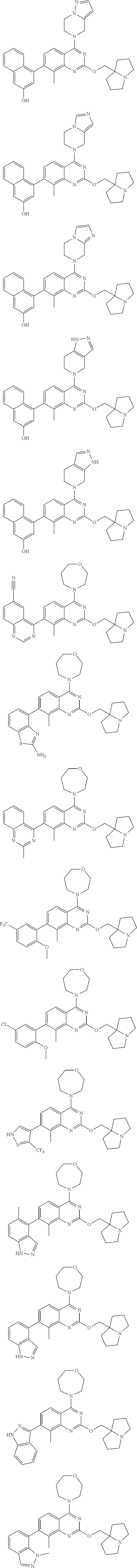

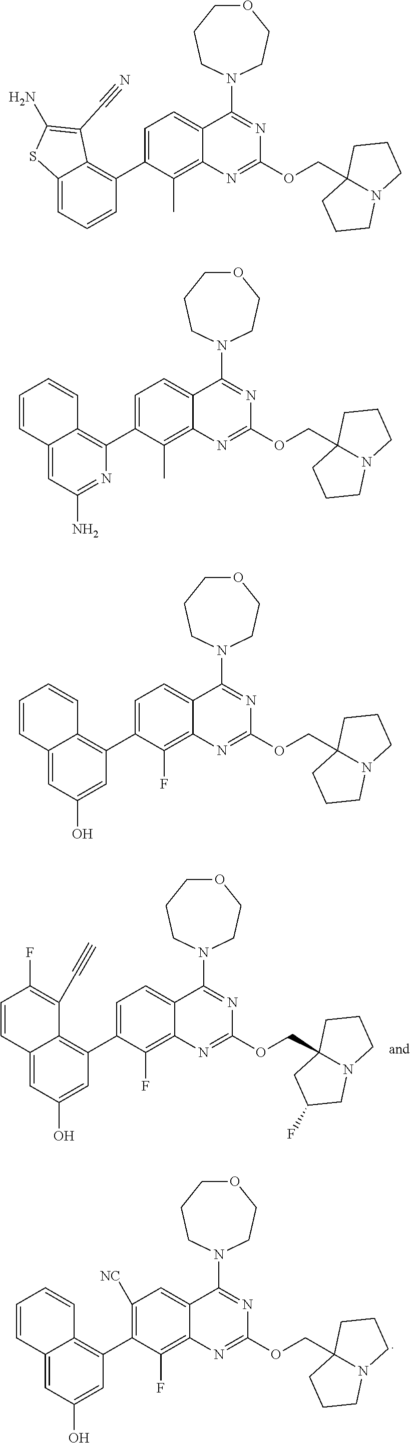 [SMILES: Cc1c(-c2cccc3sc(N)c(C#N)c23)ccc2c(N3CCCOCC3)nc(OCC34CCCN3CCC4)nc12 ; Cc1c(-c2nc(N)cc3ccccc23)ccc2c(N3CCCOCC3)nc(OCC34CCCN3CCC4)nc12 ; Oc1cc(-c2ccc3c(N4CCCOCC4)nc(OCC45CCCN4CCC5)nc3c2F)c2ccccc2c1 ; C#Cc1c(F)ccc2cc(O)cc(-c3ccc4c(N5CCCOCC5)nc(OC[C@@]56CCCN5C[C@H](F)C6)nc4c3F)c12 ; N#Cc1cc2c(N3CCCOCC3)nc(OCC34CCCN3CCC4)nc2c(F)c1-c1cc(O)cc2ccccc12]

18. A compound of any one of claims 1 to 17 for medical use.

19. A compound of any one of claims 1 to 17 for use in treating cancer.

20. A compound for use of claim 19, wherein the cancer is selected from: pancreatic carcinoma, colorectal carcinoma, rectal carcinoma, endometrial carcinoma, non-small cell lung carcinoma, gastric carcinoma, ovarian carcinoma and small cell lung carcinoma.

21. A compound for use of claim 19 or 20, wherein the subject being treated has a cancer having wild-type KRAS.

22. A compound for use of claim 19 or 20, wherein the subject being treated has a cancer having a KRAS mutation selected from: KRAS G12D, KRAS G12C, KRAS G12V, KRAS G12A, KRAS G13D and KRAS Q61H.

23. A pharmaceutical composition comprising a compound of any one of claims 1 to 17 and a pharmaceutically acceptable excipient.
